SEQUENCE LISTING (812 sequences)

SEQ ID NO: 1 - protein, 15 aa, homo sapiens
MTSPSSSPVFRLETL

SEQ ID NO: 2 - protein, 15 aa, homo sapiens
TSPSSSPVFRLETLD

SEQ ID NO: 3 - protein, 15 aa, homo sapiens
SPSSSPVFRLETLDG

SEQ ID NO: 4 - protein, 15 aa, homo sapiens
PSSSPVFRLETLDGG

SEQ ID NO: 5 - protein, 15 aa, homo sapiens
SSSPVFRLETLDGGQ

SEQ ID NO: 6 - protein, 15 aa, homo sapiens
SSPVFRLETLDGGQE

SEQ ID NO: 7 - protein, 15 aa, homo sapiens
SPVFRLETLDGGQED

SEQ ID NO: 8 - protein, 15 aa, homo sapiens
PVFRLETLDGGQEDG

SEQ ID NO: 9 - protein, 15 aa, homo sapiens
VFRLETLDGGQEDGS

SEQ ID NO: 10 - protein, 15 aa, homo sapiens
FRLETLDGGQEDGSE

SEQ ID NO: 11 - protein, 15 aa, homo sapiens
RLETLDGGQEDGSEA

SEQ ID NO: 12 - protein, 15 aa, homo sapiens
LETLDGGQEDGSEAD

SEQ ID NO: 13 - protein, 15 aa, homo sapiens
ETLDGGQEDGSEADR

SEQ ID NO: 14 - protein, 15 aa, homo sapiens
TLDGGQEDGSEADRG

SEQ ID NO: 15 - protein, 15 aa, homo sapiens
LDGGQEDGSEADRGK

SEQ ID NO: 16 - protein, 15 aa, homo sapiens
DGGQEDGSEADRGKL

SEQ ID NO: 17 - protein, 15 aa, homo sapiens
GGQEDGSEADRGKLD

SEQ ID NO: 18 - protein, 15 aa, homo sapiens
GQEDGSEADRGKLDF

SEQ ID NO: 19 - protein, 15 aa, homo sapiens
QEDGSEADRGKLDFG

SEQ ID NO: 20 - protein, 15 aa, homo sapiens
EDGSEADRGKLDFGS

SEQ ID NO: 21 - protein, 15 aa, homo sapiens
DGSEADRGKLDFGSG

SEQ ID NO: 22 - protein, 15 aa, homo sapiens
GSEADRGKLDFGSGL

SEQ ID NO: 23 - protein, 15 aa, homo sapiens
SEADRGKLDFGSGLP

SEQ ID NO: 24 - protein, 15 aa, homo sapiens
EADRGKLDFGSGLPP

SEQ ID NO: 25 - protein, 15 aa, homo sapiens
ADRGKLDFGSGLPPM

SEQ ID NO: 26 - protein, 15 aa, homo sapiens
DRGKLDFGSGLPPME

SEQ ID NO: 27 - protein, 15 aa, homo sapiens
RGKLDFGSGLPPMES

SEQ ID NO: 28 - protein, 15 aa, homo sapiens
GKLDFGSGLPPMESQ

SEQ ID NO: 29 - protein, 15 aa, homo sapiens
KLDFGSGLPPMESQF

SEQ ID NO: 30 - protein, 15 aa, homo sapiens
LDFGSGLPPMESQFQ

SEQ ID NO: 31 - protein, 15 aa, homo sapiens
DFGSGLPPMESQFQG

SEQ ID NO: 32 - protein, 15 aa, homo sapiens
FGSGLPPMESQFQGE

SEQ ID NO: 33 - protein, 15 aa, homo sapiens
GSGLPPMESQFQGED

SEQ ID NO: 34 - protein, 15 aa, homo sapiens
SGLPPMESQFQGEDR

SEQ ID NO: 35 - protein, 15 aa, homo sapiens
GLPPMESQFQGEDRK

SEQ ID NO: 36 - protein, 15 aa, homo sapiens
LPPMESQFQGEDRKF

SEQ ID NO: 37 - protein, 15 aa, homo sapiens
PPMESQFQGEDRKFA

SEQ ID NO: 38 - protein, 15 aa, homo sapiens
PMESQFQGEDRKFAP

SEQ ID NO: 39 - protein, 15 aa, homo sapiens
MESQFQGEDRKFAPQ

SEQ ID NO: 40 - protein, 15 aa, homo sapiens
ESQFQGEDRKFAPQI

SEQ ID NO: 41 - protein, 15 aa, homo sapiens
SQFQGEDRKFAPQIR

SEQ ID NO: 42 - protein, 15 aa, homo sapiens
QFQGEDRKFAPQIRV

SEQ ID NO: 43 - protein, 15 aa, homo sapiens
FQGEDRKFAPQIRVN

SEQ ID NO: 44 - protein, 15 aa, homo sapiens
QGEDRKFAPQIRVNL

SEQ ID NO: 45 - protein, 15 aa, homo sapiens
GEDRKFAPQIRVNLN

SEQ ID NO: 46 - protein, 15 aa, homo sapiens
EDRKFAPQIRVNLNY

SEQ ID NO: 47 - protein, 15 aa, homo sapiens
DRKFAPQIRVNLNYR

SEQ ID NO: 48 - protein, 15 aa, homo sapiens
RKFAPQIRVNLNYRK

SEQ ID NO: 49 - protein, 15 aa, homo sapiens
KFAPQIRVNLNYRKG

SEQ ID NO: 50 - protein, 15 aa, homo sapiens
FAPQIRVNLNYRKGT

SEQ ID NO: 51 - protein, 15 aa, homo sapiens
APQIRVNLNYRKGTG

SEQ ID NO: 52 - protein, 15 aa, homo sapiens
PQIRVNLNYRKGTGA

SEQ ID NO: 53 - protein, 15 aa, homo sapiens
QIRVNLNYRKGTGAS

SEQ ID NO: 54 - protein, 15 aa, homo sapiens
IRVNLNYRKGTGASQ

SEQ ID NO: 55 - protein, 15 aa, homo sapiens
RVNLNYRKGTGASQP

SEQ ID NO: 56 - protein, 15 aa, homo sapiens
VNLNYRKGTGASQPD

SEQ ID NO: 57 - protein, 15 aa, homo sapiens
NLNYRKGTGASQPDP

SEQ ID NO: 58 - protein, 15 aa, homo sapiens
LNYRKGTGASQPDPN

SEQ ID NO: 59 - protein, 15 aa, homo sapiens
NYRKGTGASQPDPNR

SEQ ID NO: 60 - protein, 15 aa, homo sapiens
YRKGTGASQPDPNRF

SEQ ID NO: 61 - protein, 15 aa, homo sapiens
RKGTGASQPDPNRFD

SEQ ID NO: 62 - protein, 15 aa, homo sapiens
KGTGASQPDPNRFDR

SEQ ID NO: 63 - protein, 15 aa, homo sapiens
GTGASQPDPNRFDRD

SEQ ID NO: 64 - protein, 15 aa, homo sapiens
TGASQPDPNRFDRDR

SEQ ID NO: 65 - protein, 15 aa, homo sapiens
GASQPDPNRFDRDRL

SEQ ID NO: 66 - protein, 15 aa, homo sapiens
ASQPDPNRFDRDRLF

SEQ ID NO: 67 - protein, 15 aa, homo sapiens
SQPDPNRFDRDRLFN

SEQ ID NO: 68 - protein, 15 aa, homo sapiens
QPDPNRFDRDRLFNA

SEQ ID NO: 69 - protein, 15 aa, homo sapiens
PDPNRFDRDRLFNAV

SEQ ID NO: 70 - protein, 15 aa, homo sapiens
DPNRFDRDRLFNAVS

SEQ ID NO: 71 - protein, 15 aa, homo sapiens
PNRFDRDRLFNAVSR

SEQ ID NO: 72 - protein, 15 aa, homo sapiens
NRFDRDRLFNAVSRG

SEQ ID NO: 73 - protein, 15 aa, homo sapiens
RFDRDRLFNAVSRGV

SEQ ID NO: 74 - protein, 15 aa, homo sapiens
FDRDRLFNAVSRGVP

SEQ ID NO: 75 - protein, 15 aa, homo sapiens
DRDRLFNAVSRGVPE

SEQ ID NO: 76 - protein, 15 aa, homo sapiens
RDRLFNAVSRGVPED

SEQ ID NO: 77 - protein, 15 aa, homo sapiens
DRLFNAVSRGVPEDL

SEQ ID NO: 78 - protein, 15 aa, homo sapiens
RLFNAVSRGVPEDLA

SEQ ID NO: 79 - protein, 15 aa, homo sapiens
LFNAVSRGVPEDLAG

SEQ ID NO: 80 - protein, 15 aa, homo sapiens
FNAVSRGVPEDLAGL

SEQ ID NO: 81 - protein, 15 aa, homo sapiens
NAVSRGVPEDLAGLP

SEQ ID NO: 82 - protein, 15 aa, homo sapiens
AVSRGVPEDLAGLPE

SEQ ID NO: 83 - protein, 15 aa, homo sapiens
VSRGVPEDLAGLPEY

SEQ ID NO: 84 - protein, 15 aa, homo sapiens
SRGVPEDLAGLPEYL

SEQ ID NO: 85 - protein, 15 aa, homo sapiens
RGVPEDLAGLPEYLS

SEQ ID NO: 86 - protein, 15 aa, homo sapiens
GVPEDLAGLPEYLSK

SEQ ID NO: 87 - protein, 15 aa, homo sapiens
VPEDLAGLPEYLSKT

SEQ ID NO: 88 - protein, 15 aa, homo sapiens
PEDLAGLPEYLSKTS

SEQ ID NO: 89 - protein, 15 aa, homo sapiens
EDLAGLPEYLSKTSK

SEQ ID NO: 90 - protein, 15 aa, homo sapiens
DLAGLPEYLSKTSKY

SEQ ID NO: 91 - protein, 15 aa, homo sapiens
LAGLPEYLSKTSKYL

SEQ ID NO: 92 - protein, 15 aa, homo sapiens
AGLPEYLSKTSKYLT

SEQ ID NO: 93 - protein, 15 aa, homo sapiens
GLPEYLSKTSKYLTD

SEQ ID NO: 94 - protein, 15 aa, homo sapiens
LPEYLSKTSKYLTDS

SEQ ID NO: 95 - protein, 15 aa, homo sapiens
PEYLSKTSKYLTDSE

SEQ ID NO: 96 - protein, 15 aa, homo sapiens
PEYLSKTSKYLTDSE

SEQ ID NO: 97 - protein, 15 aa, homo sapiens
EYLSKTSKYLTDSEY

SEQ ID NO: 98 - protein, 15 aa, homo sapiens
YLSKTSKYLTDSEYT

SEQ ID NO: 99 - protein, 15 aa, homo sapiens
LSKTSKYLTDSEYTE

SEQ ID NO: 100 - protein, 15 aa, homo sapiens
SKTSKYLTDSEYTEG

SEQ ID NO: 101 - protein, 15 aa, homo sapiens
KTSKYLTDSEYTEGS

SEQ ID NO: 102 - protein, 15 aa, homo sapiens
TSKYLTDSEYTEGST

SEQ ID NO: 103 - protein, 15 aa, homo sapiens
SKYLTDSEYTEGSTG

SEQ ID NO: 104 - protein, 15 aa, homo sapiens
KYLTDSEYTEGSTGK

SEQ ID NO: 105 - protein, 15 aa, homo sapiens
YLTDSEYTEGSTGKT

SEQ ID NO: 106 - protein, 15 aa, homo sapiens
LTDSEYTEGSTGKTC

SEQ ID NO: 107 - protein, 15 aa, homo sapiens
TDSEYTEGSTGKTCL

SEQ ID NO: 108 - protein, 15 aa, homo sapiens
DSEYTEGSTGKTCLM

SEQ ID NO: 109 - protein, 15 aa, homo sapiens
SEYTEGSTGKTCLMK

SEQ ID NO: 110 - protein, 15 aa, homo sapiens
EYTEGSTGKTCLMKA

SEQ ID NO: 111 - protein, 15 aa, homo sapiens
YTEGSTGKTCLMKAV

SEQ ID NO: 112 - protein, 15 aa, homo sapiens
TEGSTGKTCLMKAVL

SEQ ID NO: 113 - protein, 15 aa, homo sapiens
EGSTGKTCLMKAVLN

SEQ ID NO: 114 - protein, 15 aa, homo sapiens
GSTGKTCLMKAVLNL

SEQ ID NO: 115 - protein, 15 aa, homo sapiens
STGKTCLMKAVLNLK

SEQ ID NO: 116 - protein, 15 aa, homo sapiens
TGKTCLMKAVLNLKD

SEQ ID NO: 117 - protein, 15 aa, homo sapiens
GKTCLMKAVLNLKDG

SEQ ID NO: 118 - protein, 15 aa, homo sapiens
KTCLMKAVLNLKDGV

SEQ ID NO: 119 - protein, 15 aa, homo sapiens
TCLMKAVLNLKDGVN

SEQ ID NO: 120 - protein, 15 aa, homo sapiens
CLMKAVLNLKDGVNA

SEQ ID NO: 121 - protein, 15 aa, homo sapiens
LMKAVLNLKDGVNAC

SEQ ID NO: 122 - protein, 15 aa, homo sapiens
MKAVLNLKDGVNACI

SEQ ID NO: 123 - protein, 15 aa, homo sapiens
KAVLNLKDGVNACIL

SEQ ID NO: 124 - protein, 15 aa, homo sapiens
AVLNLKDGVNACILP

SEQ ID NO: 125 - protein, 15 aa, homo sapiens
VLNLKDGVNACILPL

SEQ ID NO: 126 - protein, 15 aa, homo sapiens
LNLKDGVNACILPLL

SEQ ID NO: 127 - protein, 15 aa, homo sapiens
NLKDGVNACILPLLQ

SEQ ID NO: 128 - protein, 15 aa, homo sapiens
LKDGVNACILPLLQI

SEQ ID NO: 129 - protein, 15 aa, homo sapiens
KDGVNACILPLLQID

SEQ ID NO: 130 - protein, 15 aa, homo sapiens
DGVNACILPLLQIDR

SEQ ID NO: 131 - protein, 15 aa, homo sapiens
GVNACILPLLQIDRD

SEQ ID NO: 132 - protein, 15 aa, homo sapiens
VNACILPLLQIDRDS

SEQ ID NO: 133 - protein, 15 aa, homo sapiens
NACILPLLQIDRDSG

SEQ ID NO: 134 - protein, 15 aa, homo sapiens
ACILPLLQIDRDSGN

SEQ ID NO: 135 - protein, 15 aa, homo sapiens
CILPLLQIDRDSGNP

SEQ ID NO: 136 - protein, 15 aa, homo sapiens
ILPLLQIDRDSGNPQ

SEQ ID NO: 137 - protein, 15 aa, homo sapiens
LPLLQIDRDSGNPQP

SEQ ID NO: 138 - protein, 15 aa, homo sapiens
PLLQIDRDSGNPQPL

SEQ ID NO: 139 - protein, 15 aa, homo sapiens
LLQIDRDSGNPQPLV

SEQ ID NO: 140 - protein, 15 aa, homo sapiens
LQIDRDSGNPQPLVN

SEQ ID NO: 141 - protein, 15 aa, homo sapiens
QIDRDSGNPQPLVNA

SEQ ID NO: 142 - protein, 15 aa, homo sapiens
IDRDSGNPQPLVNAQ

SEQ ID NO: 143 - protein, 15 aa, homo sapiens
DRDSGNPQPLVNAQC

SEQ ID NO: 144 - protein, 15 aa, homo sapiens
RDSGNPQPLVNAQCT

SEQ ID NO: 145 - protein, 15 aa, homo sapiens
DSGNPQPLVNAQCTD

SEQ ID NO: 146 - protein, 15 aa, homo sapiens
SGNPQPLVNAQCTDD

SEQ ID NO: 147 - protein, 15 aa, homo sapiens
GNPQPLVNAQCTDDY

SEQ ID NO: 148 - protein, 15 aa, homo sapiens
NPQPLVNAQCTDDYY

SEQ ID NO: 149 - protein, 15 aa, homo sapiens
PQPLVNAQCTDDYYR

SEQ ID NO: 150 - protein, 15 aa, homo sapiens
QPLVNAQCTDDYYRG

SEQ ID NO: 151 - protein, 15 aa, homo sapiens
PLVNAQCTDDYYRGH

SEQ ID NO: 152 - protein, 15 aa, homo sapiens
LVNAQCTDDYYRGHS

SEQ ID NO: 153 - protein, 15 aa, homo sapiens
VNAQCTDDYYRGHSA

SEQ ID NO: 154 - protein, 15 aa, homo sapiens
NAQCTDDYYRGHSAL

SEQ ID NO: 155 - protein, 15 aa, homo sapiens
AQCTDDYYRGHSALH

SEQ ID NO: 156 - protein, 15 aa, homo sapiens
QCTDDYYRGHSALHI

SEQ ID NO: 157 - protein, 15 aa, homo sapiens
CTDDYYRGHSALHIA

SEQ ID NO: 158 - protein, 15 aa, homo sapiens
TDDYYRGHSALHIAI

SEQ ID NO: 159 - protein, 15 aa, homo sapiens
DDYYRGHSALHIAIE

SEQ ID NO: 160 - protein, 15 aa, homo sapiens
DYYRGHSALHIAIEK

SEQ ID NO: 161 - protein, 15 aa, homo sapiens
YYRGHSALHIAIEKR

SEQ ID NO: 162 - protein, 15 aa, homo sapiens
YRGHSALHIAIEKRS

SEQ ID NO: 163 - protein, 15 aa, homo sapiens
RGHSALHIAIEKRSL

SEQ ID NO: 164 - protein, 15 aa, homo sapiens
GHSALHIAIEKRSLQ

SEQ ID NO: 165 - protein, 15 aa, homo sapiens
HSALHIAIEKRSLQC

SEQ ID NO: 166 - protein, 15 aa, homo sapiens
SALHIAIEKRSLQCV

SEQ ID NO: 167 - protein, 15 aa, homo sapiens
ALHIAIEKRSLQCVK

SEQ ID NO: 168 - protein, 15 aa, homo sapiens
LHIAIEKRSLQCVKL

SEQ ID NO: 169 - protein, 15 aa, homo sapiens
HIAIEKRSLQCVKLL

SEQ ID NO: 170 - protein, 15 aa, homo sapiens
IAIEKRSLQCVKLLV

SEQ ID NO: 171 - protein, 15 aa, homo sapiens
AIEKRSLQCVKLLVE

SEQ ID NO: 172 - protein, 15 aa, homo sapiens
IEKRSLQCVKLLVEN

SEQ ID NO: 173 - protein, 15 aa, homo sapiens
EKRSLQCVKLLVENG

SEQ ID NO: 174 - protein, 15 aa, homo sapiens
KRSLQCVKLLVENGA

SEQ ID NO: 175 - protein, 15 aa, homo sapiens
RSLQCVKLLVENGAN

SEQ ID NO: 176 - protein, 15 aa, homo sapiens
SLQCVKLLVENGANV

SEQ ID NO: 177 - protein, 15 aa, homo sapiens
LQCVKLLVENGANVH

SEQ ID NO: 178 - protein, 15 aa, homo sapiens
QCVKLLVENGANVHA

SEQ ID NO: 179 - protein, 15 aa, homo sapiens
CVKLLVENGANVHAR

SEQ ID NO: 180 - protein, 15 aa, homo sapiens
VKLLVENGANVHARA

SEQ ID NO: 181 - protein, 15 aa, homo sapiens
KLLVENGANVHARAC

SEQ ID NO: 182 - protein, 15 aa, homo sapiens
LLVENGANVHARACG

SEQ ID NO: 183 - protein, 15 aa, homo sapiens
LVENGANVHARACGR

SEQ ID NO: 184 - protein, 15 aa, homo sapiens
VENGANVHARACGRF

SEQ ID NO: 185 - protein, 15 aa, homo sapiens
ENGANVHARACGRFF

SEQ ID NO: 186 - protein, 15 aa, homo sapiens
NGANVHARACGRFFQ

SEQ ID NO: 187 - protein, 15 aa, homo sapiens
GANVHARACGRFFQK

SEQ ID NO: 188 - protein, 15 aa, homo sapiens
ANVHARACGRFFQKG

SEQ ID NO: 189 - protein, 15 aa, homo sapiens
NVHARACGRFFQKGQ

SEQ ID NO: 190 - protein, 15 aa, homo sapiens
VHARACGRFFQKGQG

SEQ ID NO: 191 - protein, 15 aa, homo sapiens
HARACGRFFQKGQGT

SEQ ID NO: 192 - protein, 15 aa, homo sapiens
ARACGRFFQKGQGTC

SEQ ID NO: 193 - protein, 15 aa, homo sapiens
RACGRFFQKGQGTCF

SEQ ID NO: 194 - protein, 15 aa, homo sapiens
ACGRFFQKGQGTCFY

SEQ ID NO: 195 - protein, 15 aa, homo sapiens
CGRFFQKGQGTCFYF

SEQ ID NO: 196 - protein, 15 aa, homo sapiens
GRFFQKGQGTCFYFG

SEQ ID NO: 197 - protein, 15 aa, homo sapiens
RFFQKGQGTCFYFGE

SEQ ID NO: 198 - protein, 15 aa, homo sapiens
FFQKGQGTCFYFGEL

SEQ ID NO: 199 - protein, 15 aa, homo sapiens
FQKGQGTCFYFGELP

SEQ ID NO: 200 - protein, 15 aa, homo sapiens
QKGQGTCFYFGELPL

SEQ ID NO: 201 - protein, 15 aa, homo sapiens
KGQGTCFYFGELPLS

SEQ ID NO: 202 - protein, 15 aa, homo sapiens
GQGTCFYFGELPLSL

SEQ ID NO: 203 - protein, 15 aa, homo sapiens
QGTCFYFGELPLSLA

SEQ ID NO: 204 - protein, 15 aa, homo sapiens
GTCFYFGELPLSLAA

SEQ ID NO: 205 - protein, 15 aa, homo sapiens
TCFYFGELPLSLAAC

SEQ ID NO: 206 - protein, 15 aa, homo sapiens
CFYFGELPLSLAACT

SEQ ID NO: 207 - protein, 15 aa, homo sapiens
FYFGELPLSLAACTK

SEQ ID NO: 208 - protein, 15 aa, homo sapiens
YFGELPLSLAACTKQ

SEQ ID NO: 209 - protein, 15 aa, homo sapiens
FGELPLSLAACTKQW

SEQ ID NO: 210 - protein, 15 aa, homo sapiens
GELPLSLAACTKQWD

SEQ ID NO: 211 - protein, 15 aa, homo sapiens
ELPLSLAACTKQWDV

SEQ ID NO: 212 - protein, 15 aa, homo sapiens
LPLSLAACTKQWDVV

SEQ ID NO: 213 - protein, 15 aa, homo sapiens
PLSLAACTKQWDVVS

SEQ ID NO: 214 - protein, 15 aa, homo sapiens
LSLAACTKQWDVVSY

SEQ ID NO: 215 - protein, 15 aa, homo sapiens
SLAACTKQWDVVSYL

SEQ ID NO: 216 - protein, 15 aa, homo sapiens
LAACTKQWDVVSYLL

SEQ ID NO: 217 - protein, 15 aa, homo sapiens
AACTKQWDVVSYLLE

SEQ ID NO: 218 - protein, 15 aa, homo sapiens
ACTKQWDVVSYLLEN

SEQ ID NO: 219 - protein, 15 aa, homo sapiens
CTKQWDVVSYLLENP

SEQ ID NO: 220 - protein, 15 aa, homo sapiens
TKQWDVVSYLLENPH

SEQ ID NO: 221 - protein, 15 aa, homo sapiens
KQWDVVSYLLENPHQ

SEQ ID NO: 222 - protein, 15 aa, homo sapiens
QWDVVSYLLENPHQP

SEQ ID NO: 223 - protein, 15 aa, homo sapiens
WDVVSYLLENPHQPA

SEQ ID NO: 224 - protein, 15 aa, homo sapiens
DVVSYLLENPHQPAS

SEQ ID NO: 225 - protein, 15 aa, homo sapiens
VVSYLLENPHQPASL

SEQ ID NO: 226 - protein, 15 aa, homo sapiens
VSYLLENPHQPASLQ

SEQ ID NO: 227 - protein, 15 aa, homo sapiens
SYLLENPHQPASLQA

SEQ ID NO: 228 - protein, 15 aa, homo sapiens
YLLENPHQPASLQAT

SEQ ID NO: 229 - protein, 15 aa, homo sapiens
LLENPHQPASLQATD

SEQ ID NO: 230 - protein, 15 aa, homo sapiens
LENPHQPASLQATDS

SEQ ID NO: 231 - protein, 15 aa, homo sapiens
ENPHQPASLQATDSQ

SEQ ID NO: 232 - protein, 15 aa, homo sapiens
NPHQPASLQATDSQG

SEQ ID NO: 233 - protein, 15 aa, homo sapiens
PHQPASLQATDSQGN

SEQ ID NO: 234 - protein, 15 aa, homo sapiens
HQPASLQATDSQGNT

SEQ ID NO: 235 - protein, 15 aa, homo sapiens
QPASLQATDSQGNTV

SEQ ID NO: 236 - protein, 15 aa, homo sapiens
PASLQATDSQGNTVL

SEQ ID NO: 237 - protein, 15 aa, homo sapiens
ASLQATDSQGNTVLH

SEQ ID NO: 238 - protein, 15 aa, homo sapiens
SLQATDSQGNTVLHA

SEQ ID NO: 239 - protein, 15 aa, homo sapiens
LQATDSQGNTVLHAL

SEQ ID NO: 240 - protein, 15 aa, homo sapiens
QATDSQGNTVLHALV

SEQ ID NO: 241 - protein, 15 aa, homo sapiens
ATDSQGNTVLHALVM

SEQ ID NO: 242 - protein, 15 aa, homo sapiens
TDSQGNTVLHALVMI

SEQ ID NO: 243 - protein, 15 aa, homo sapiens
DSQGNTVLHALVMIS

SEQ ID NO: 244 - protein, 15 aa, homo sapiens
SQGNTVLHALVMISD

SEQ ID NO: 245 - protein, 15 aa, homo sapiens
QGNTVLHALVMISDN

SEQ ID NO: 246 - protein, 15 aa, homo sapiens
GNTVLHALVMISDNS

SEQ ID NO: 247 - protein, 15 aa, homo sapiens
NTVLHALVMISDNSA

SEQ ID NO: 248 - protein, 15 aa, homo sapiens
TVLHALVMISDNSAE

SEQ ID NO: 249 - protein, 15 aa, homo sapiens
VLHALVMISDNSAEN

SEQ ID NO: 250 - protein, 15 aa, homo sapiens
LHALVMISDNSAENI

SEQ ID NO: 251 - protein, 15 aa, homo sapiens
HALVMISDNSAENIA

SEQ ID NO: 252 - protein, 15 aa, homo sapiens
ALVMISDNSAENIAL

SEQ ID NO: 253 - protein, 15 aa, homo sapiens
LVMISDNSAENIALV

SEQ ID NO: 254 - protein, 15 aa, homo sapiens
VMISDNSAENIALVT

SEQ ID NO: 255 - protein, 15 aa, homo sapiens
MISDNSAENIALVTS

SEQ ID NO: 256 - protein, 15 aa, homo sapiens
ISDNSAENIALVTSM

SEQ ID NO: 257 - protein, 15 aa, homo sapiens
SDNSAENIALVTSMY

SEQ ID NO: 258 - protein, 15 aa, homo sapiens
DNSAENIALVTSMYD

SEQ ID NO: 259 - protein, 15 aa, homo sapiens
NSAENIALVTSMYDG

SEQ ID NO: 260 - protein, 15 aa, homo sapiens
SAENIALVTSMYDGL

SEQ ID NO: 261 - protein, 15 aa, homo sapiens
AENIALVTSMYDGLL

SEQ ID NO: 262 - protein, 15 aa, homo sapiens
ENIALVTSMYDGLLQ

SEQ ID NO: 263 - protein, 15 aa, homo sapiens
NIALVTSMYDGLLQA

SEQ ID NO: 264 - protein, 15 aa, homo sapiens
IALVTSMYDGLLQAG

SEQ ID NO: 265 - protein, 15 aa, homo sapiens
ALVTSMYDGLLQAGA

SEQ ID NO: 266 - protein, 15 aa, homo sapiens
LVTSMYDGLLQAGAR

SEQ ID NO: 267 - protein, 15 aa, homo sapiens
VTSMYDGLLQAGARL

SEQ ID NO: 268 - protein, 15 aa, homo sapiens
TSMYDGLLQAGARLC

SEQ ID NO: 269 - protein, 15 aa, homo sapiens
SMYDGLLQAGARLCP

SEQ ID NO: 270 - protein, 15 aa, homo sapiens
MYDGLLQAGARLCPT

SEQ ID NO: 271 - protein, 15 aa, homo sapiens
YDGLLQAGARLCPTV

SEQ ID NO: 272 - protein, 15 aa, homo sapiens
DGLLQAGARLCPTVQ

SEQ ID NO: 273 - protein, 15 aa, homo sapiens
GLLQAGARLCPTVQL

SEQ ID NO: 274 - protein, 15 aa, homo sapiens
LLQAGARLCPTVQLE

SEQ ID NO: 275 - protein, 15 aa, homo sapiens
LQAGARLCPTVQLED

SEQ ID NO: 276 - protein, 15 aa, homo sapiens
QAGARLCPTVQLEDI

SEQ ID NO: 277 - protein, 15 aa, homo sapiens
AGARLCPTVQLEDIR

SEQ ID NO: 278 - protein, 15 aa, homo sapiens
GARLCPTVQLEDIRN

SEQ ID NO: 279 - protein, 15 aa, homo sapiens
ARLCPTVQLEDIRNL

SEQ ID NO: 280 - protein, 15 aa, homo sapiens
RLCPTVQLEDIRNLQ

SEQ ID NO: 281 - protein, 15 aa, homo sapiens
LCPTVQLEDIRNLQD

SEQ ID NO: 282 - protein, 15 aa, homo sapiens
CPTVQLEDIRNLQDL

SEQ ID NO: 283 - protein, 15 aa, homo sapiens
PTVQLEDIRNLQDLT

SEQ ID NO: 284 - protein, 15 aa, homo sapiens
TVQLEDIRNLQDLTP

SEQ ID NO: 285 - protein, 15 aa, homo sapiens
VQLEDIRNLQDLTPL

SEQ ID NO: 286 - protein, 15 aa, homo sapiens
QLEDIRNLQDLTPLK

SEQ ID NO: 287 - protein, 15 aa, homo sapiens
LEDIRNLQDLTPLKL

SEQ ID NO: 288 - protein, 15 aa, homo sapiens
EDIRNLQDLTPLKLA

SEQ ID NO: 289 - protein, 15 aa, homo sapiens
DIRNLQDLTPLKLAA

SEQ ID NO: 290 - protein, 15 aa, homo sapiens
IRNLQDLTPLKLAAK

SEQ ID NO: 291 - protein, 15 aa, homo sapiens
RNLQDLTPLKLAAKE

SEQ ID NO: 292 - protein, 15 aa, homo sapiens
NLQDLTPLKLAAKEG

SEQ ID NO: 293 - protein, 15 aa, homo sapiens
LQDLTPLKLAAKEGK

SEQ ID NO: 294 - protein, 15 aa, homo sapiens
QDLTPLKLAAKEGKI

SEQ ID NO: 295 - protein, 15 aa, homo sapiens
DLTPLKLAAKEGKIE

SEQ ID NO: 296 - protein, 15 aa, homo sapiens
LTPLKLAAKEGKIEI

SEQ ID NO: 297 - protein, 15 aa, homo sapiens
TPLKLAAKEGKIEIF

SEQ ID NO: 298 - protein, 15 aa, homo sapiens
PLKLAAKEGKIEIFR

SEQ ID NO: 299 - protein, 15 aa, homo sapiens
LKLAAKEGKIEIFRH

SEQ ID NO: 300 - protein, 15 aa, homo sapiens
KLAAKEGKIEIFRHI

SEQ ID NO: 301 - protein, 15 aa, homo sapiens
LAAKEGKIEIFRHIL

SEQ ID NO: 302 - protein, 15 aa, homo sapiens
AAKEGKIEIFRHILQ

SEQ ID NO: 303 - protein, 15 aa, homo sapiens
AKEGKIEIFRHILQR

SEQ ID NO: 304 - protein, 15 aa, homo sapiens
KEGKIEIFRHILQRE

SEQ ID NO: 305 - protein, 15 aa, homo sapiens
EGKIEIFRHILQREF

SEQ ID NO: 306 - protein, 15 aa, homo sapiens
GKIEIFRHILQREFS

SEQ ID NO: 307 - protein, 15 aa, homo sapiens
KIEIFRHILQREFSG

SEQ ID NO: 308 - protein, 15 aa, homo sapiens
IEIFRHILQREFSGL

SEQ ID NO: 309 - protein, 15 aa, homo sapiens
EIFRHILQREFSGLS

SEQ ID NO: 310 - protein, 15 aa, homo sapiens
IFRHILQREFSGLSH

SEQ ID NO: 311 - protein, 15 aa, homo sapiens
FRHILQREFSGLSHL

SEQ ID NO: 312 - protein, 15 aa, homo sapiens
RHILQREFSGLSHLS

SEQ ID NO: 313 - protein, 15 aa, homo sapiens
HILQREFSGLSHLSR

SEQ ID NO: 314 - protein, 15 aa, homo sapiens
ILQREFSGLSHLSRK

SEQ ID NO: 315 - protein, 15 aa, homo sapiens
LQREFSGLSHLSRKF

SEQ ID NO: 316 - protein, 15 aa, homo sapiens
QREFSGLSHLSRKFT

SEQ ID NO: 317 - protein, 15 aa, homo sapiens
REFSGLSHLSRKFTE

SEQ ID NO: 318 - protein, 15 aa, homo sapiens
EFSGLSHLSRKFTEW

SEQ ID NO: 319 - protein, 15 aa, homo sapiens
FSGLSHLSRKFTEWC

SEQ ID NO: 320 - protein, 15 aa, homo sapiens
SGLSHLSRKFTEWCY

SEQ ID NO: 321 - protein, 15 aa, homo sapiens
GLSHLSRKFTEWCYG

SEQ ID NO: 322 - protein, 15 aa, homo sapiens
LSHLSRKFTEWCYGP

SEQ ID NO: 323 - protein, 15 aa, homo sapiens
SHLSRKFTEWCYGPV

SEQ ID NO: 324 - protein, 15 aa, homo sapiens
HLSRKFTEWCYGPVR

SEQ ID NO: 325 - protein, 15 aa, homo sapiens
LSRKFTEWCYGPVRV

SEQ ID NO: 326 - protein, 15 aa, homo sapiens
SRKFTEWCYGPVRVS

SEQ ID NO: 327 - protein, 15 aa, homo sapiens
RKFTEWCYGPVRVSL

SEQ ID NO: 328 - protein, 15 aa, homo sapiens
KFTEWCYGPVRVSLY

SEQ ID NO: 329 - protein, 15 aa, homo sapiens
FTEWCYGPVRVSLYD

SEQ ID NO: 330 - protein, 15 aa, homo sapiens
TEWCYGPVRVSLYDL

SEQ ID NO: 331 - protein, 15 aa, homo sapiens
EWCYGPVRVSLYDLA

SEQ ID NO: 332 - protein, 15 aa, homo sapiens
WCYGPVRVSLYDLAS

SEQ ID NO: 333 - protein, 15 aa, homo sapiens
CYGPVRVSLYDLASV

SEQ ID NO: 334 - protein, 15 aa, homo sapiens
YGPVRVSLYDLASVD

SEQ ID NO: 335 - protein, 15 aa, homo sapiens
GPVRVSLYDLASVDS

SEQ ID NO: 336 - protein, 15 aa, homo sapiens
PVRVSLYDLASVDSC

SEQ ID NO: 337 - protein, 15 aa, homo sapiens
VRVSLYDLASVDSCE

SEQ ID NO: 338 - protein, 15 aa, homo sapiens
RVSLYDLASVDSCEE

SEQ ID NO: 339 - protein, 15 aa, homo sapiens
VSLYDLASVDSCEEN

SEQ ID NO: 340 - protein, 15 aa, homo sapiens
SLYDLASVDSCEENS

SEQ ID NO: 341 - protein, 15 aa, homo sapiens
LYDLASVDSCEENSV

SEQ ID NO: 342 - protein, 15 aa, homo sapiens
YDLASVDSCEENSVL

SEQ ID NO: 343 - protein, 15 aa, homo sapiens
DLASVDSCEENSVLE

SEQ ID NO: 344 - protein, 15 aa, homo sapiens
LASVDSCEENSVLEI

SEQ ID NO: 345 - protein, 15 aa, homo sapiens
ASVDSCEENSVLEII

SEQ ID NO: 346 - protein, 15 aa, homo sapiens
SVDSCEENSVLEIIA

SEQ ID NO: 347 - protein, 15 aa, homo sapiens
VDSCEENSVLEIIAF

SEQ ID NO: 348 - protein, 15 aa, homo sapiens
DSCEENSVLEIIAFH

SEQ ID NO: 349 - protein, 15 aa, homo sapiens
SCEENSVLEIIAFHC

SEQ ID NO: 350 - protein, 15 aa, homo sapiens
CEENSVLEIIAFHCK

SEQ ID NO: 351 - protein, 15 aa, homo sapiens
EENSVLEIIAFHCKS

SEQ ID NO: 352 - protein, 15 aa, homo sapiens
ENSVLEIIAFHCKSP

SEQ ID NO: 353 - protein, 15 aa, homo sapiens
NSVLEIIAFHCKSPH

SEQ ID NO: 354 - protein, 15 aa, homo sapiens
SVLEIIAFHCKSPHR

SEQ ID NO: 355 - protein, 15 aa, homo sapiens
VLEIIAFHCKSPHRH

SEQ ID NO: 356 - protein, 15 aa, homo sapiens
LEIIAFHCKSPHRHR

SEQ ID NO: 357 - protein, 15 aa, homo sapiens
EIIAFHCKSPHRHRM

SEQ ID NO: 358 - protein, 15 aa, homo sapiens
IIAFHCKSPHRHRMV

SEQ ID NO: 359 - protein, 15 aa, homo sapiens
IAFHCKSPHRHRMVV

SEQ ID NO: 360 - protein, 15 aa, homo sapiens
AFHCKSPHRHRMVVL

SEQ ID NO: 361 - protein, 15 aa, homo sapiens
FHCKSPHRHRMVVLE

SEQ ID NO: 362 - protein, 15 aa, homo sapiens
HCKSPHRHRMVVLEP

SEQ ID NO: 363 - protein, 15 aa, homo sapiens
CKSPHRHRMVVLEPL

SEQ ID NO: 364 - protein, 15 aa, homo sapiens
KSPHRHRMVVLEPLN

SEQ ID NO: 365 - protein, 15 aa, homo sapiens
SPHRHRMVVLEPLNK

SEQ ID NO: 366 - protein, 15 aa, homo sapiens
PHRHRMVVLEPLNKL

SEQ ID NO: 367 - protein, 15 aa, homo sapiens
HRHRMVVLEPLNKLL

SEQ ID NO: 368 - protein, 15 aa, homo sapiens
RHRMVVLEPLNKLLQ

SEQ ID NO: 369 - protein, 15 aa, homo sapiens
HRMVVLEPLNKLLQA

SEQ ID NO: 370 - protein, 15 aa, homo sapiens
RMVVLEPLNKLLQAK

SEQ ID NO: 371 - protein, 15 aa, homo sapiens
MVVLEPLNKLLQAKW

SEQ ID NO: 372 - protein, 15 aa, homo sapiens
VVLEPLNKLLQAKWD

SEQ ID NO: 373 - protein, 15 aa, homo sapiens
VLEPLNKLLQAKWDL

SEQ ID NO: 374 - protein, 15 aa, homo sapiens
LEPLNKLLQAKWDLL

SEQ ID NO: 375 - protein, 15 aa, homo sapiens
EPLNKLLQAKWDLLI

SEQ ID NO: 376 - protein, 15 aa, homo sapiens
PLNKLLQAKWDLLIP

SEQ ID NO: 377 - protein, 15 aa, homo sapiens
LNKLLQAKWDLLIPK

SEQ ID NO: 378 - protein, 15 aa, homo sapiens
NKLLQAKWDLLIPKF

SEQ ID NO: 379 - protein, 15 aa, homo sapiens
KLLQAKWDLLIPKFF

SEQ ID NO: 380 - protein, 15 aa, homo sapiens
LLQAKWDLLIPKFFL

SEQ ID NO: 381 - protein, 15 aa, homo sapiens
LQAKWDLLIPKFFLN

SEQ ID NO: 382 - protein, 15 aa, homo sapiens
QAKWDLLIPKFFLNF

SEQ ID NO: 383 - protein, 15 aa, homo sapiens
AKWDLLIPKFFLNFL

SEQ ID NO: 384 - protein, 15 aa, homo sapiens
KWDLLIPKFFLNFLC

SEQ ID NO: 385 - protein, 15 aa, homo sapiens
WDLLIPKFFLNFLCN

SEQ ID NO: 386 - protein, 15 aa, homo sapiens
DLLIPKFFLNFLCNL

SEQ ID NO: 387 - protein, 15 aa, homo sapiens
LLIPKFFLNFLCNLI

SEQ ID NO: 388 - protein, 15 aa, homo sapiens
LIPKFFLNFLCNLIY

SEQ ID NO: 389 - protein, 15 aa, homo sapiens
IPKFFLNFLCNLIYM

SEQ ID NO: 390 - protein, 15 aa, homo sapiens
PKFFLNFLCNLIYMF

SEQ ID NO: 391 - protein, 15 aa, homo sapiens
KFFLNFLCNLIYMFI

SEQ ID NO: 392 - protein, 15 aa, homo sapiens
FFLNFLCNLIYMFIF

SEQ ID NO: 393 - protein, 15 aa, homo sapiens
FLNFLCNLIYMFIFT

SEQ ID NO: 394 - protein, 15 aa, homo sapiens
LNFLCNLIYMFIFTA

SEQ ID NO: 395 - protein, 15 aa, homo sapiens
NFLCNLIYMFIFTAV

SEQ ID NO: 396 - protein, 15 aa, homo sapiens
FLCNLIYMFIFTAVA

SEQ ID NO: 397 - protein, 15 aa, homo sapiens
LCNLIYMFIFTAVAY

SEQ ID NO: 398 - protein, 15 aa, homo sapiens
CNLIYMFIFTAVAYH

SEQ ID NO: 399 - protein, 15 aa, homo sapiens
NLIYMFIFTAVAYHQ

SEQ ID NO: 400 - protein, 15 aa, homo sapiens
LIYMFIFTAVAYHQP

SEQ ID NO: 401 - protein, 15 aa, homo sapiens
IYMFIFTAVAYHQPT

SEQ ID NO: 402 - protein, 15 aa, homo sapiens
YMFIFTAVAYHQPTL

SEQ ID NO: 403 - protein, 15 aa, homo sapiens
MFIFTAVAYHQPTLK

SEQ ID NO: 404 - protein, 15 aa, homo sapiens
FIFTAVAYHQPTLKK

SEQ ID NO: 405 - protein, 15 aa, homo sapiens
IFTAVAYHQPTLKKQ

SEQ ID NO: 406 - protein, 15 aa, homo sapiens
FTAVAYHQPTLKKQA

SEQ ID NO: 407 - protein, 15 aa, homo sapiens
TAVAYHQPTLKKQAA

SEQ ID NO: 408 - protein, 15 aa, homo sapiens
AVAYHQPTLKKQAAP

SEQ ID NO: 409 - protein, 15 aa, homo sapiens
VAYHQPTLKKQAAPH

SEQ ID NO: 410 - protein, 15 aa, homo sapiens
AYHQPTLKKQAAPHL

SEQ ID NO: 411 - protein, 15 aa, homo sapiens
YHQPTLKKQAAPHLK

SEQ ID NO: 412 - protein, 15 aa, homo sapiens
HQPTLKKQAAPHLKA

SEQ ID NO: 413 - protein, 15 aa, homo sapiens
QPTLKKQAAPHLKAE

SEQ ID NO: 414 - protein, 15 aa, homo sapiens
PTLKKQAAPHLKAEV

SEQ ID NO: 415 - protein, 15 aa, homo sapiens
TLKKQAAPHLKAEVG

SEQ ID NO: 416 - protein, 15 aa, homo sapiens
LKKQAAPHLKAEVGN

SEQ ID NO: 417 - protein, 15 aa, homo sapiens
KKQAAPHLKAEVGNS

SEQ ID NO: 418 - protein, 15 aa, homo sapiens
KQAAPHLKAEVGNSM

SEQ ID NO: 419 - protein, 15 aa, homo sapiens
QAAPHLKAEVGNSML

SEQ ID NO: 420 - protein, 15 aa, homo sapiens
AAPHLKAEVGNSMLL

SEQ ID NO: 421 - protein, 15 aa, homo sapiens
APHLKAEVGNSMLLT

SEQ ID NO: 422 - protein, 15 aa, homo sapiens
PHLKAEVGNSMLLTG

SEQ ID NO: 423 - protein, 15 aa, homo sapiens
HLKAEVGNSMLLTGH

SEQ ID NO: 424 - protein, 15 aa, homo sapiens
LKAEVGNSMLLTGHI

SEQ ID NO: 425 - protein, 15 aa, homo sapiens
KAEVGNSMLLTGHIL

SEQ ID NO: 426 - protein, 15 aa, homo sapiens
AEVGNSMLLTGHILI

SEQ ID NO: 427 - protein, 15 aa, homo sapiens
EVGNSMLLTGHILIL

SEQ ID NO: 428 - protein, 15 aa, homo sapiens
VGNSMLLTGHILILL

SEQ ID NO: 429 - protein, 15 aa, homo sapiens
GNSMLLTGHILILLG

SEQ ID NO: 430 - protein, 15 aa, homo sapiens
NSMLLTGHILILLGG

SEQ ID NO: 431 - protein, 15 aa, homo sapiens
SMLLTGHILILLGGI

SEQ ID NO: 432 - protein, 15 aa, homo sapiens
MLLTGHILILLGGIY

SEQ ID NO: 433 - protein, 15 aa, homo sapiens
LLTGHILILLGGIYL

SEQ ID NO: 434 - protein, 15 aa, homo sapiens
LTGHILILLGGIYLL

SEQ ID NO: 435 - protein, 15 aa, homo sapiens
TGHILILLGGIYLLV

SEQ ID NO: 436 - protein, 15 aa, homo sapiens
GHILILLGGIYLLVG

SEQ ID NO: 437 - protein, 15 aa, homo sapiens
HILILLGGIYLLVGQ

SEQ ID NO: 438 - protein, 15 aa, homo sapiens
ILILLGGIYLLVGQL

SEQ ID NO: 439 - protein, 15 aa, homo sapiens
LILLGGIYLLVGQLW

SEQ ID NO: 440 - protein, 15 aa, homo sapiens
ILLGGIYLLVGQLWY

SEQ ID NO: 441 - protein, 15 aa, homo sapiens
LLGGIYLLVGQLWYF

SEQ ID NO: 442 - protein, 15 aa, homo sapiens
LGGIYLLVGQLWYFW

SEQ ID NO: 443 - protein, 15 aa, homo sapiens
GGIYLLVGQLWYFWR

SEQ ID NO: 444 - protein, 15 aa, homo sapiens
GIYLLVGQLWYFWRR

SEQ ID NO: 445 - protein, 15 aa, homo sapiens
IYLLVGQLWYFWRRH

SEQ ID NO: 446 - protein, 15 aa, homo sapiens
YLLVGQLWYFWRRHV

SEQ ID NO: 447 - protein, 15 aa, homo sapiens
LLVGQLWYFWRRHVF

SEQ ID NO: 448 - protein, 15 aa, homo sapiens
LVGQLWYFWRRHVFI

SEQ ID NO: 449 - protein, 15 aa, homo sapiens
VGQLWYFWRRHVFIW

SEQ ID NO: 450 - protein, 15 aa, homo sapiens
GQLWYFWRRHVFIWI

SEQ ID NO: 451 - protein, 15 aa, homo sapiens
QLWYFWRRHVFIWIS

SEQ ID NO: 452 - protein, 15 aa, homo sapiens
LWYFWRRHVFIWISF

SEQ ID NO: 453 - protein, 15 aa, homo sapiens
WYFWRRHVFIWISFI

SEQ ID NO: 454 - protein, 15 aa, homo sapiens
YFWRRHVFIWISFID

SEQ ID NO: 455 - protein, 15 aa, homo sapiens
FWRRHVFIWISFIDS

SEQ ID NO: 456 - protein, 15 aa, homo sapiens
WRRHVFIWISFIDSY

SEQ ID NO: 457 - protein, 15 aa, homo sapiens
RRHVFIWISFIDSYF

SEQ ID NO: 458 - protein, 15 aa, homo sapiens
RHVFIWISFIDSYFE

SEQ ID NO: 459 - protein, 15 aa, homo sapiens
HVFIWISFIDSYFEI

SEQ ID NO: 460 - protein, 15 aa, homo sapiens
VFIWISFIDSYFEIL

SEQ ID NO: 461 - protein, 15 aa, homo sapiens
FIWISFIDSYFEILF

SEQ ID NO: 462 - protein, 15 aa, homo sapiens
IWISFIDSYFEILFL

SEQ ID NO: 463 - protein, 15 aa, homo sapiens
WISFIDSYFEILFLF

SEQ ID NO: 464 - protein, 15 aa, homo sapiens
ISFIDSYFEILFLFQ

SEQ ID NO: 465 - protein, 15 aa, homo sapiens
SFIDSYFEILFLFQA

SEQ ID NO: 466 - protein, 15 aa, homo sapiens
FIDSYFEILFLFQAL

SEQ ID NO: 467 - protein, 15 aa, homo sapiens
IDSYFEILFLFQALL

SEQ ID NO: 468 - protein, 15 aa, homo sapiens
DSYFEILFLFQALLT

SEQ ID NO: 469 - protein, 15 aa, homo sapiens
SYFEILFLFQALLTV

SEQ ID NO: 470 - protein, 15 aa, homo sapiens
YFEILFLFQALLTVV

SEQ ID NO: 471 - protein, 15 aa, homo sapiens
FEILFLFQALLTVVS

SEQ ID NO: 472 - protein, 15 aa, homo sapiens
EILFLFQALLTVVSQ

SEQ ID NO: 473 - protein, 15 aa, homo sapiens
ILFLFQALLTVVSQV

SEQ ID NO: 474 - protein, 15 aa, homo sapiens
LFLFQALLTVVSQVL

SEQ ID NO: 475 - protein, 15 aa, homo sapiens
FLFQALLTVVSQVLC

SEQ ID NO: 476 - protein, 15 aa, homo sapiens
LFQALLTVVSQVLCF

SEQ ID NO: 477 - protein, 15 aa, homo sapiens
FQALLTVVSQVLCFL

SEQ ID NO: 478 - protein, 15 aa, homo sapiens
QALLTVVSQVLCFLA

SEQ ID NO: 479 - protein, 15 aa, homo sapiens
ALLTVVSQVLCFLAI

SEQ ID NO: 480 - protein, 15 aa, homo sapiens
LLTVVSQVLCFLAIE

SEQ ID NO: 481 - protein, 15 aa, homo sapiens
LTVVSQVLCFLAIEW

SEQ ID NO: 482 - protein, 15 aa, homo sapiens
TVVSQVLCFLAIEWY

SEQ ID NO: 483 - protein, 15 aa, homo sapiens
VVSQVLCFLAIEWYL

SEQ ID NO: 484 - protein, 15 aa, homo sapiens
VSQVLCFLAIEWYLP

SEQ ID NO: 485 - protein, 15 aa, homo sapiens
SQVLCFLAIEWYLPL

SEQ ID NO: 486 - protein, 15 aa, homo sapiens
QVLCFLAIEWYLPLL

SEQ ID NO: 487 - protein, 15 aa, homo sapiens
VLCFLAIEWYLPLLV

SEQ ID NO: 488 - protein, 15 aa, homo sapiens
LCFLAIEWYLPLLVS

SEQ ID NO: 489 - protein, 15 aa, homo sapiens
CFLAIEWYLPLLVSA

SEQ ID NO: 490 - protein, 15 aa, homo sapiens
FLAIEWYLPLLVSAL

SEQ ID NO: 491 - protein, 15 aa, homo sapiens
LAIEWYLPLLVSALV

SEQ ID NO: 492 - protein, 15 aa, homo sapiens
AIEWYLPLLVSALVL

SEQ ID NO: 493 - protein, 15 aa, homo sapiens
IEWYLPLLVSALVLG

SEQ ID NO: 494 - protein, 15 aa, homo sapiens
EWYLPLLVSALVLGW

SEQ ID NO: 495 - protein, 15 aa, homo sapiens
WYLPLLVSALVLGWL

SEQ ID NO: 496 - protein, 15 aa, homo sapiens
YLPLLVSALVLGWLN

SEQ ID NO: 497 - protein, 15 aa, homo sapiens
LPLLVSALVLGWLNL

SEQ ID NO: 498 - protein, 15 aa, homo sapiens
PLLVSALVLGWLNLL

SEQ ID NO: 499 - protein, 15 aa, homo sapiens
LLVSALVLGWLNLLY

SEQ ID NO: 500 - protein, 15 aa, homo sapiens
LVSALVLGWLNLLYY

SEQ ID NO: 501 - protein, 15 aa, homo sapiens
VSALVLGWLNLLYYT

SEQ ID NO: 502 - protein, 15 aa, homo sapiens
SALVLGWLNLLYYTR

SEQ ID NO: 503 - protein, 15 aa, homo sapiens
ALVLGWLNLLYYTRG

SEQ ID NO: 504 - protein, 15 aa, homo sapiens
LVLGWLNLLYYTRGF

SEQ ID NO: 505 - protein, 15 aa, homo sapiens
VLGWLNLLYYTRGFQ

SEQ ID NO: 506 - protein, 15 aa, homo sapiens
LGWLNLLYYTRGFQH

SEQ ID NO: 507 - protein, 15 aa, homo sapiens
GWLNLLYYTRGFQHT

SEQ ID NO: 508 - protein, 15 aa, homo sapiens
WLNLLYYTRGFQHTG

SEQ ID NO: 509 - protein, 15 aa, homo sapiens
LNLLYYTRGFQHTGI

SEQ ID NO: 510 - protein, 15 aa, homo sapiens
NLLYYTRGFQHTGIY

SEQ ID NO: 511 - protein, 15 aa, homo sapiens
LLYYTRGFQHTGIYS

SEQ ID NO: 512 - protein, 15 aa, homo sapiens
LYYTRGFQHTGIYSV

SEQ ID NO: 513 - protein, 15 aa, homo sapiens
YYTRGFQHTGIYSVM

SEQ ID NO: 514 - protein, 15 aa, homo sapiens
YTRGFQHTGIYSVMI

SEQ ID NO: 515 - protein, 15 aa, homo sapiens
TRGFQHTGIYSVMIQ

SEQ ID NO: 516 - protein, 15 aa, homo sapiens
RGFQHTGIYSVMIQK

SEQ ID NO: 517 - protein, 15 aa, homo sapiens
GFQHTGIYSVMIQKV

SEQ ID NO: 518 - protein, 15 aa, homo sapiens
FQHTGIYSVMIQKVI

SEQ ID NO: 519 - protein, 15 aa, homo sapiens
QHTGIYSVMIQKVIL

SEQ ID NO: 520 - protein, 15 aa, homo sapiens
HTGIYSVMIQKVILR

SEQ ID NO: 521 - protein, 15 aa, homo sapiens
TGIYSVMIQKVILRD

SEQ ID NO: 522 - protein, 15 aa, homo sapiens
GIYSVMIQKVILRDL

SEQ ID NO: 523 - protein, 15 aa, homo sapiens
IYSVMIQKVILRDLL

SEQ ID NO: 524 - protein, 15 aa, homo sapiens
YSVMIQKVILRDLLR

SEQ ID NO: 525 - protein, 15 aa, homo sapiens
SVMIQKVILRDLLRF

SEQ ID NO: 526 - protein, 15 aa, homo sapiens
VMIQKVILRDLLRFL

SEQ ID NO: 527 - protein, 15 aa, homo sapiens
MIQKVILRDLLRFLL

SEQ ID NO: 528 - protein, 15 aa, homo sapiens
IQKVILRDLLRFLLI

SEQ ID NO: 529 - protein, 15 aa, homo sapiens
QKVILRDLLRFLLIY

SEQ ID NO: 530 - protein, 15 aa, homo sapiens
KVILRDLLRFLLIYL

SEQ ID NO: 531 - protein, 15 aa, homo sapiens
VILRDLLRFLLIYLV

SEQ ID NO: 532 - protein, 15 aa, homo sapiens
ILRDLLRFLLIYLVF

SEQ ID NO: 533 - protein, 15 aa, homo sapiens
LRDLLRFLLIYLVFL

SEQ ID NO: 534 - protein, 15 aa, homo sapiens
RDLLRFLLIYLVFLF

SEQ ID NO: 535 - protein, 15 aa, homo sapiens
DLLRFLLIYLVFLFG

SEQ ID NO: 536 - protein, 15 aa, homo sapiens
LLRFLLIYLVFLFGF

SEQ ID NO: 537 - protein, 15 aa, homo sapiens
LRFLLIYLVFLFGFA

SEQ ID NO: 538 - protein, 15 aa, homo sapiens
RFLLIYLVFLFGFAV

SEQ ID NO: 539 - protein, 15 aa, homo sapiens
FLLIYLVFLFGFAVA

SEQ ID NO: 540 - protein, 15 aa, homo sapiens
LLIYLVFLFGFAVAL

SEQ ID NO: 541 - protein, 15 aa, homo sapiens
LIYLVFLFGFAVALV

SEQ ID NO: 542 - protein, 15 aa, homo sapiens
IYLVFLFGFAVALVS

SEQ ID NO: 543 - protein, 15 aa, homo sapiens
YLVFLFGFAVALVSL

SEQ ID NO: 544 - protein, 15 aa, homo sapiens
LVFLFGFAVALVSLS

SEQ ID NO: 545 - protein, 15 aa, homo sapiens
VFLFGFAVALVSLSQ

SEQ ID NO: 546 - protein, 15 aa, homo sapiens
FLFGFAVALVSLSQE

SEQ ID NO: 547 - protein, 15 aa, homo sapiens
LFGFAVALVSLSQEA

SEQ ID NO: 548 - protein, 15 aa, homo sapiens
FGFAVALVSLSQEAW

SEQ ID NO: 549 - protein, 15 aa, homo sapiens
GFAVALVSLSQEAWR

SEQ ID NO: 550 - protein, 15 aa, homo sapiens
FAVALVSLSQEAWRP

SEQ ID NO: 551 - protein, 15 aa, homo sapiens
AVALVSLSQEAWRPE

SEQ ID NO: 552 - protein, 15 aa, homo sapiens
VALVSLSQEAWRPEA

SEQ ID NO: 553 - protein, 15 aa, homo sapiens
ALVSLSQEAWRPEAP

SEQ ID NO: 554 - protein, 15 aa, homo sapiens
LVSLSQEAWRPEAPT

SEQ ID NO: 555 - protein, 15 aa, homo sapiens
VSLSQEAWRPEAPTG

SEQ ID NO: 556 - protein, 15 aa, homo sapiens
SLSQEAWRPEAPTGP

SEQ ID NO: 557 - protein, 15 aa, homo sapiens
LSQEAWRPEAPTGPN

SEQ ID NO: 558 - protein, 15 aa, homo sapiens
SQEAWRPEAPTGPNA

SEQ ID NO: 559 - protein, 15 aa, homo sapiens
QEAWRPEAPTGPNAT

SEQ ID NO: 560 - protein, 15 aa, homo sapiens
EAWRPEAPTGPNATE

SEQ ID NO: 561 - protein, 15 aa, homo sapiens
AWRPEAPTGPNATES

SEQ ID NO: 562 - protein, 15 aa, homo sapiens
WRPEAPTGPNATESV

SEQ ID NO: 563 - protein, 15 aa, homo sapiens
RPEAPTGPNATESVQ

SEQ ID NO: 564 - protein, 15 aa, homo sapiens
PEAPTGPNATESVQP

SEQ ID NO: 565 - protein, 15 aa, homo sapiens
EAPTGPNATESVQPM

SEQ ID NO: 566 - protein, 15 aa, homo sapiens
APTGPNATESVQPME

SEQ ID NO: 567 - protein, 15 aa, homo sapiens
PTGPNATESVQPMEG

SEQ ID NO: 568 - protein, 15 aa, homo sapiens
TGPNATESVQPMEGQ

SEQ ID NO: 569 - protein, 15 aa, homo sapiens
GPNATESVQPMEGQE

SEQ ID NO: 570 - protein, 15 aa, homo sapiens
PNATESVQPMEGQED

SEQ ID NO: 571 - protein, 15 aa, homo sapiens
NATESVQPMEGQEDE

SEQ ID NO: 572 - protein, 15 aa, homo sapiens
ATESVQPMEGQEDEG

SEQ ID NO: 573 - protein, 15 aa, homo sapiens
TESVQPMEGQEDEGN

SEQ ID NO: 574 - protein, 15 aa, homo sapiens
ESVQPMEGQEDEGNG

SEQ ID NO: 575 - protein, 15 aa, homo sapiens
SVQPMEGQEDEGNGA

SEQ ID NO: 576 - protein, 15 aa, homo sapiens
VQPMEGQEDEGNGAQ

SEQ ID NO: 577 - protein, 15 aa, homo sapiens
QPMEGQEDEGNGAQY

SEQ ID NO: 578 - protein, 15 aa, homo sapiens
PMEGQEDEGNGAQYR

SEQ ID NO: 579 - protein, 15 aa, homo sapiens
MEGQEDEGNGAQYRG

SEQ ID NO: 580 - protein, 15 aa, homo sapiens
EGQEDEGNGAQYRGI

SEQ ID NO: 581 - protein, 15 aa, homo sapiens
GQEDEGNGAQYRGIL

SEQ ID NO: 582 - protein, 15 aa, homo sapiens
QEDEGNGAQYRGILE

SEQ ID NO: 583 - protein, 15 aa, homo sapiens
EDEGNGAQYRGILEA

SEQ ID NO: 584 - protein, 15 aa, homo sapiens
DEGNGAQYRGILEAS

SEQ ID NO: 585 - protein, 15 aa, homo sapiens
EGNGAQYRGILEASL

SEQ ID NO: 586 - protein, 15 aa, homo sapiens
GNGAQYRGILEASLE

SEQ ID NO: 587 - protein, 15 aa, homo sapiens
NGAQYRGILEASLEL

SEQ ID NO: 588 - protein, 15 aa, homo sapiens
GAQYRGILEASLELF

SEQ ID NO: 589 - protein, 15 aa, homo sapiens
AQYRGILEASLELFK

SEQ ID NO: 590 - protein, 15 aa, homo sapiens
QYRGILEASLELFKF

SEQ ID NO: 591 - protein, 15 aa, homo sapiens
YRGILEASLELFKFT

SEQ ID NO: 592 - protein, 15 aa, homo sapiens
RGILEASLELFKFTI

SEQ ID NO: 593 - protein, 15 aa, homo sapiens
GILEASLELFKFTIG

SEQ ID NO: 594 - protein, 15 aa, homo sapiens
ILEASLELFKFTIGM

SEQ ID NO: 595 - protein, 15 aa, homo sapiens
LEASLELFKFTIGMG

SEQ ID NO: 596 - protein, 15 aa, homo sapiens
EASLELFKFTIGMGE

SEQ ID NO: 597 - protein, 15 aa, homo sapiens
ASLELFKFTIGMGEL

SEQ ID NO: 598 - protein, 15 aa, homo sapiens
SLELFKFTIGMGELA

SEQ ID NO: 599 - protein, 15 aa, homo sapiens
LELFKFTIGMGELAF

SEQ ID NO: 600 - protein, 15 aa, homo sapiens
ELFKFTIGMGELAFQ

SEQ ID NO: 601 - protein, 15 aa, homo sapiens
LFKFTIGMGELAFQE

SEQ ID NO: 602 - protein, 15 aa, homo sapiens
FKFTIGMGELAFQEQ

SEQ ID NO: 603 - protein, 15 aa, homo sapiens
KFTIGMGELAFQEQL

SEQ ID NO: 604 - protein, 15 aa, homo sapiens
FTIGMGELAFQEQLH

SEQ ID NO: 605 - protein, 15 aa, homo sapiens
TIGMGELAFQEQLHF

SEQ ID NO: 606 - protein, 15 aa, homo sapiens
IGMGELAFQEQLHFR

SEQ ID NO: 607 - protein, 15 aa, homo sapiens
GMGELAFQEQLHFRG

SEQ ID NO: 608 - protein, 15 aa, homo sapiens
MGELAFQEQLHFRGM

SEQ ID NO: 609 - protein, 15 aa, homo sapiens
GELAFQEQLHFRGMV

SEQ ID NO: 610 - protein, 15 aa, homo sapiens
ELAFQEQLHFRGMVL

SEQ ID NO: 611 - protein, 15 aa, homo sapiens
LAFQEQLHFRGMVLL

SEQ ID NO: 612 - protein, 15 aa, homo sapiens
AFQEQLHFRGMVLLL

SEQ ID NO: 613 - protein, 15 aa, homo sapiens
FQEQLHFRGMVLLLL

SEQ ID NO: 614 - protein, 15 aa, homo sapiens
QEQLHFRGMVLLLLL

SEQ ID NO: 615 - protein, 15 aa, homo sapiens
EQLHFRGMVLLLLLA

SEQ ID NO: 616 - protein, 15 aa, homo sapiens
QLHFRGMVLLLLLAY

SEQ ID NO: 617 - protein, 15 aa, homo sapiens
LHFRGMVLLLLLAYV

SEQ ID NO: 618 - protein, 15 aa, homo sapiens
HFRGMVLLLLLAYVL

SEQ ID NO: 619 - protein, 15 aa, homo sapiens
FRGMVLLLLLAYVLL

SEQ ID NO: 620 - protein, 15 aa, homo sapiens
RGMVLLLLLAYVLLT

SEQ ID NO: 621 - protein, 15 aa, homo sapiens
GMVLLLLLAYVLLTY

SEQ ID NO: 622 - protein, 15 aa, homo sapiens
MVLLLLLAYVLLTYI

SEQ ID NO: 623 - protein, 15 aa, homo sapiens
VLLLLLAYVLLTYIL

SEQ ID NO: 624 - protein, 15 aa, homo sapiens
LLLLLAYVLLTYILL

SEQ ID NO: 625 - protein, 15 aa, homo sapiens
LLLLAYVLLTYILLL

SEQ ID NO: 626 - protein, 15 aa, homo sapiens
LLLAYVLLTYILLLN

SEQ ID NO: 627 - protein, 15 aa, homo sapiens
LLAYVLLTYILLLNM

SEQ ID NO: 628 - protein, 15 aa, homo sapiens
LAYVLLTYILLLNML

SEQ ID NO: 629 - protein, 15 aa, homo sapiens
AYVLLTYILLLNMLI

SEQ ID NO: 630 - protein, 15 aa, homo sapiens
YVLLTYILLLNMLIA

SEQ ID NO: 631 - protein, 15 aa, homo sapiens
VLLTYILLLNMLIAL

SEQ ID NO: 632 - protein, 15 aa, homo sapiens
LLTYILLLNMLIALM

SEQ ID NO: 633 - protein, 15 aa, homo sapiens
LTYILLLNMLIALMS

SEQ ID NO: 634 - protein, 15 aa, homo sapiens
TYILLLNMLIALMSE

SEQ ID NO: 635 - protein, 15 aa, homo sapiens
YILLLNMLIALMSET

SEQ ID NO: 636 - protein, 15 aa, homo sapiens
ILLLNMLIALMSETV

SEQ ID NO: 637 - protein, 15 aa, homo sapiens
LLLNMLIALMSETVN

SEQ ID NO: 638 - protein, 15 aa, homo sapiens
LLNMLIALMSETVNS

SEQ ID NO: 639 - protein, 15 aa, homo sapiens
LNMLIALMSETVNSV

SEQ ID NO: 640 - protein, 15 aa, homo sapiens
NMLIALMSETVNSVA

SEQ ID NO: 641 - protein, 15 aa, homo sapiens
MLIALMSETVNSVAT

SEQ ID NO: 642 - protein, 15 aa, homo sapiens
LIALMSETVNSVATD

SEQ ID NO: 643 - protein, 15 aa, homo sapiens
IALMSETVNSVATDS

SEQ ID NO: 644 - protein, 15 aa, homo sapiens
ALMSETVNSVATDSW

SEQ ID NO: 645 - protein, 15 aa, homo sapiens
LMSETVNSVATDSWS

SEQ ID NO: 646 - protein, 15 aa, homo sapiens
MSETVNSVATDSWSI

SEQ ID NO: 647 - protein, 15 aa, homo sapiens
SETVNSVATDSWSIW

SEQ ID NO: 648 - protein, 15 aa, homo sapiens
ETVNSVATDSWSIWK

SEQ ID NO: 649 - protein, 15 aa, homo sapiens
TVNSVATDSWSIWKL

SEQ ID NO: 650 - protein, 15 aa, homo sapiens
VNSVATDSWSIWKLQ

SEQ ID NO: 651 - protein, 15 aa, homo sapiens
NSVATDSWSIWKLQK

SEQ ID NO: 652 - protein, 15 aa, homo sapiens
SVATDSWSIWKLQKA

SEQ ID NO: 653 - protein, 15 aa, homo sapiens
VATDSWSIWKLQKAI

SEQ ID NO: 654 - protein, 15 aa, homo sapiens
ATDSWSIWKLQKAIS

SEQ ID NO: 655 - protein, 15 aa, homo sapiens
TDSWSIWKLQKAISV

SEQ ID NO: 656 - protein, 15 aa, homo sapiens
DSWSIWKLQKAISVL

SEQ ID NO: 657 - protein, 15 aa, homo sapiens
SWSIWKLQKAISVLE

SEQ ID NO: 658 - protein, 15 aa, homo sapiens
WSIWKLQKAISVLEM

SEQ ID NO: 659 - protein, 15 aa, homo sapiens
SIWKLQKAISVLEME

SEQ ID NO: 660 - protein, 15 aa, homo sapiens
IWKLQKAISVLEMEN

SEQ ID NO: 661 - protein, 15 aa, homo sapiens
WKLQKAISVLEMENG

SEQ ID NO: 662 - protein, 15 aa, homo sapiens
KLQKAISVLEMENGY

SEQ ID NO: 663 - protein, 15 aa, homo sapiens
LQKAISVLEMENGYW

SEQ ID NO: 664 - protein, 15 aa, homo sapiens
QKAISVLEMENGYWW

SEQ ID NO: 665 - protein, 15 aa, homo sapiens
KAISVLEMENGYWWC

SEQ ID NO: 666 - protein, 15 aa, homo sapiens
AISVLEMENGYWWCR

SEQ ID NO: 667 - protein, 15 aa, homo sapiens
ISVLEMENGYWWCRK

SEQ ID NO: 668 - protein, 15 aa, homo sapiens
SVLEMENGYWWCRKK

SEQ ID NO: 669 - protein, 15 aa, homo sapiens
VLEMENGYWWCRKKQ

SEQ ID NO: 670 - protein, 15 aa, homo sapiens
LEMENGYWWCRKKQR

SEQ ID NO: 671 - protein, 15 aa, homo sapiens
EMENGYWWCRKKQRA

SEQ ID NO: 672 - protein, 15 aa, homo sapiens
MENGYWWCRKKQRAG

SEQ ID NO: 673 - protein, 15 aa, homo sapiens
ENGYWWCRKKQRAGV

SEQ ID NO: 674 - protein, 15 aa, homo sapiens
NGYWWCRKKQRAGVM

SEQ ID NO: 675 - protein, 15 aa, homo sapiens
GYWWCRKKQRAGVML

SEQ ID NO: 676 - protein, 15 aa, homo sapiens
YWWCRKKQRAGVMLT

SEQ ID NO: 677 - protein, 15 aa, homo sapiens
WWCRKKQRAGVMLTV

SEQ ID NO: 678 - protein, 15 aa, homo sapiens
WCRKKQRAGVMLTVG

SEQ ID NO: 679 - protein, 15 aa, homo sapiens
CRKKQRAGVMLTVGT

SEQ ID NO: 680 - protein, 15 aa, homo sapiens
RKKQRAGVMLTVGTK

SEQ ID NO: 681 - protein, 15 aa, homo sapiens
KKQRAGVMLTVGTKP

SEQ ID NO: 682 - protein, 15 aa, homo sapiens
KQRAGVMLTVGTKPD

SEQ ID NO: 683 - protein, 15 aa, homo sapiens
QRAGVMLTVGTKPDG

SEQ ID NO: 684 - protein, 15 aa, homo sapiens
RAGVMLTVGTKPDGS

SEQ ID NO: 685 - protein, 15 aa, homo sapiens
AGVMLTVGTKPDGSP

SEQ ID NO: 686 - protein, 15 aa, homo sapiens
GVMLTVGTKPDGSPD

SEQ ID NO: 687 - protein, 15 aa, homo sapiens
VMLTVGTKPDGSPDE

SEQ ID NO: 688 - protein, 15 aa, homo sapiens
MLTVGTKPDGSPDER

SEQ ID NO: 689 - protein, 15 aa, homo sapiens
LTVGTKPDGSPDERW

SEQ ID NO: 690 - protein, 15 aa, homo sapiens
TVGTKPDGSPDERWC

SEQ ID NO: 691 - protein, 15 aa, homo sapiens
VGTKPDGSPDERWCF

SEQ ID NO: 692 - protein, 15 aa, homo sapiens
GTKPDGSPDERWCFR

SEQ ID NO: 693 - protein, 15 aa, homo sapiens
TKPDGSPDERWCFRV

SEQ ID NO: 694 - protein, 15 aa, homo sapiens
KPDGSPDERWCFRVE

SEQ ID NO: 695 - protein, 15 aa, homo sapiens
PDGSPDERWCFRVEE

SEQ ID NO: 696 - protein, 15 aa, homo sapiens
DGSPDERWCFRVEEV

SEQ ID NO: 697 - protein, 15 aa, homo sapiens
GSPDERWCFRVEEVN

SEQ ID NO: 698 - protein, 15 aa, homo sapiens
SPDERWCFRVEEVNW

SEQ ID NO: 699 - protein, 15 aa, homo sapiens
PDERWCFRVEEVNWA

SEQ ID NO: 700 - protein, 15 aa, homo sapiens
DERWCFRVEEVNWAS

SEQ ID NO: 701 - protein, 15 aa, homo sapiens
ERWCFRVEEVNWASW

SEQ ID NO: 702 - protein, 15 aa, homo sapiens
RWCFRVEEVNWASWE

SEQ ID NO: 703 - protein, 15 aa, homo sapiens
WCFRVEEVNWASWEQ

SEQ ID NO: 704 - protein, 15 aa, homo sapiens
CFRVEEVNWASWEQT

SEQ ID NO: 705 - protein, 15 aa, homo sapiens
FRVEEVNWASWEQTL

SEQ ID NO: 706 - protein, 15 aa, homo sapiens
RVEEVNWASWEQTLP

SEQ ID NO: 707 - protein, 15 aa, homo sapiens
VEEVNWASWEQTLPT

SEQ ID NO: 708 - protein, 15 aa, homo sapiens
EEVNWASWEQTLPTL

SEQ ID NO: 709 - protein, 15 aa, homo sapiens
EVNWASWEQTLPTLC

SEQ ID NO: 710 - protein, 15 aa, homo sapiens
VNWASWEQTLPTLCE

SEQ ID NO: 711 - protein, 15 aa, homo sapiens
NWASWEQTLPTLCED

SEQ ID NO: 712 - protein, 15 aa, homo sapiens
WASWEQTLPTLCEDP

SEQ ID NO: 713 - protein, 15 aa, homo sapiens
ASWEQTLPTLCEDPS

SEQ ID NO: 714 - protein, 15 aa, homo sapiens
SWEQTLPTLCEDPSG

SEQ ID NO: 715 - protein, 15 aa, homo sapiens
WEQTLPTLCEDPSGA

SEQ ID NO: 716 - protein, 15 aa, homo sapiens
EQTLPTLCEDPSGAG

SEQ ID NO: 717 - protein, 15 aa, homo sapiens
QTLPTLCEDPSGAGV

SEQ ID NO: 718 - protein, 15 aa, homo sapiens
TLPTLCEDPSGAGVP

SEQ ID NO: 719 - protein, 15 aa, homo sapiens
LPTLCEDPSGAGVPR

SEQ ID NO: 720 - protein, 15 aa, homo sapiens
PTLCEDPSGAGVPRT

SEQ ID NO: 721 - protein, 15 aa, homo sapiens
TLCEDPSGAGVPRTL

SEQ ID NO: 722 - protein, 15 aa, homo sapiens
LCEDPSGAGVPRTLE

SEQ ID NO: 723 - protein, 15 aa, homo sapiens
CEDPSGAGVPRTLEN

SEQ ID NO: 724 - protein, 15 aa, homo sapiens
EDPSGAGVPRTLENP

SEQ ID NO: 725 - protein, 15 aa, homo sapiens
DPSGAGVPRTLENPV

SEQ ID NO: 726 - protein, 15 aa, homo sapiens
PSGAGVPRTLENPVL

SEQ ID NO: 727 - protein, 15 aa, homo sapiens
SGAGVPRTLENPVLA

SEQ ID NO: 728 - protein, 15 aa, homo sapiens
GAGVPRTLENPVLAS

SEQ ID NO: 729 - protein, 15 aa, homo sapiens
AGVPRTLENPVLASP

SEQ ID NO: 730 - protein, 15 aa, homo sapiens
GVPRTLENPVLASPP

SEQ ID NO: 731 - protein, 15 aa, homo sapiens
VPRTLENPVLASPPK

SEQ ID NO: 732 - protein, 15 aa, homo sapiens
PRTLENPVLASPPKE

SEQ ID NO: 733 - protein, 15 aa, homo sapiens
RTLENPVLASPPKED

SEQ ID NO: 734 - protein, 15 aa, homo sapiens
TLENPVLASPPKEDE

SEQ ID NO: 735 - protein, 15 aa, homo sapiens
LENPVLASPPKEDED

SEQ ID NO: 736 - protein, 15 aa, homo sapiens
ENPVLASPPKEDEDG

SEQ ID NO: 737 - protein, 15 aa, homo sapiens
NPVLASPPKEDEDGA

SEQ ID NO: 738 - protein, 15 aa, homo sapiens
PVLASPPKEDEDGAS

SEQ ID NO: 739 - protein, 15 aa, homo sapiens
VLASPPKEDEDGASE

SEQ ID NO: 740 - protein, 15 aa, homo sapiens
LASPPKEDEDGASEE

SEQ ID NO: 741 - protein, 15 aa, homo sapiens
ASPPKEDEDGASEEN

SEQ ID NO: 742 - protein, 15 aa, homo sapiens
SPPKEDEDGASEENY

SEQ ID NO: 743 - protein, 15 aa, homo sapiens
PPKEDEDGASEENYV

SEQ ID NO: 744 - protein, 15 aa, homo sapiens
PKEDEDGASEENYVP

SEQ ID NO: 745 - protein, 15 aa, homo sapiens
KEDEDGASEENYVPV

SEQ ID NO: 746 - protein, 15 aa, homo sapiens
EDEDGASEENYVPVQ

SEQ ID NO: 747 - protein, 15 aa, homo sapiens
DEDGASEENYVPVQL

SEQ ID NO: 748 - protein, 15 aa, homo sapiens
EDGASEENYVPVQLL

SEQ ID NO: 749 - protein, 15 aa, homo sapiens
DGASEENYVPVQLLQ

SEQ ID NO: 750 - protein, 15 aa, homo sapiens
GASEENYVPVQLLQS

SEQ ID NO: 751 - protein, 15 aa, homo sapiens
ASEENYVPVQLLQSN

SEQ ID NO: 752 - protein, 764 aa, homo sapiens
MTSPSSSPVFRLETLDGGQEDGSEADRGKLDFGSGLPPMESQFQGEDRKFAPQIRVNLNYRKGTGASQPDPNRFDRDRLFNAVSRGVPEDLAGLPEYLSKTSKYLTDSEYTEGSTGKTCLMKAVLNLKDGVNACILPLLQIDRDSGNPQPLVNAQCTDDYYRGHSALHIAIEKRSLQCVKLLVENGANVHARACGRFFQKGQGTCFYFGELPLSLAACTKQWDVVSYLLENPHQPASLQATDSQGNTVLHALVMISDNSAENIALVTSMYDGLLQAGARLCPTVQLEDIRNLQDLTPLKLAAKEGKIEIFRHILQREFSGLSHLSRKFTEWCYGPVRVSLYDLASVDSCEENSVLEIIAFHCKSPHRHRMVVLEPLNKLLQAKWDLLIPKFFLNFLCNLIYMFIFTAVAYHQPTLKKQAAPHLKAEVGNSMLLTGHILILLGGIYLLVGQLWYFWRRHVFIWISFIDSYFEILFLFQALLTVVSQVLCFLAIEWYLPLLVSALVLGWLNLLYYTRGFQHTGIYSVMIQKVILRDLLRFLLIYLVFLFGFAVALVSLSQEAWRPEAPTGPNATESVQPMEGQEDEGNGAQYRGILEASLELFKFTIGMGELAFQEQLHFRGMVLLLLLAYVLLTYILLLNMLIALMSETVNSVATDSWSIWKLQKAISVLEMENGYWWCRKKQRAGVMLTVGTKPDGSPDERWCFRVEEVNWASWEQTLPTLCEDPSGAGVPRTLENPVLASPPKEDEDGASEENYVPVQLLQSN

SEQ ID NO: 753 - DNA, 19 bp, Artificial Sequence
  Chemically Synthesized
gcctgggctgtttctccaa

SEQ ID NO: 754 - DNA, 20 bp, Artificial Sequence
  Chemically Synthesized
cgtgagtagccggcagagag

SEQ ID NO: 755 - DNA, 19 bp, Artificial Sequence
  Chemically Synthesized
gtgcaaagtggccatgagg

SEQ ID NO: 756 - DNA, 21 bp, Artificial Sequence
  Chemically Synthesized
gcactggtgaagtttcggaag

SEQ ID NO: 757 - DNA, 20 bp, Artificial Sequence
  Chemically Synthesized
gccaatcagtgtgcaagcaa

SEQ ID NO: 758 - DNA, 19 bp, Artificial Sequence
  Chemically Synthesized
gaagcctcagacggccatt

SEQ ID NO: 759 - DNA, 19 bp, Artificial Sequence
  Chemically Synthesized
gccaacttccggagcttca

SEQ ID NO: 760 - DNA, 19 bp, Artificial Sequence
  Chemically Synthesized
ggcacgagagcagcatgtc

SEQ ID NO: 761 - DNA, 20 bp, Artificial Sequence
  Chemically Synthesized
catcacagcctgtcggattg

SEQ ID NO: 762 - DNA, 20 bp, Artificial Sequence
  Chemically Synthesized
gcaggctctgcacactcctt

SEQ ID NO: 763 - DNA, 19 bp, Artificial Sequence
  Chemically Synthesized
cccagtccgacctttgaca

SEQ ID NO: 764 - DNA, 20 bp, Artificial Sequence
  Chemically Synthesized
tcggcaatccaacaacatgt

SEQ ID NO: 765 - DNA, 21 bp, Artificial Sequence
  Chemically Synthesized
aacctgcttcccaaccatcac

SEQ ID NO: 766 - DNA, 20 bp, Artificial Sequence
  Chemically Synthesized
ctttccccacacagcaccat

SEQ ID NO: 767 - DNA, 20 bp, Artificial Sequence
  Chemically Synthesized
ctgggctctgtctcgctctt

SEQ ID NO: 768 - DNA, 20 bp, Artificial Sequence
  Chemically Synthesized
gttgtcgttggccacatgaa

SEQ ID NO: 769 - DNA, 21 bp, Artificial Sequence
  Chemically Synthesized
tgccataaggcctctgaatga

SEQ ID NO: 770 - DNA, 21 bp, Artificial Sequence
  Chemically Synthesized
ctcagttaggcgaaccctgaa

SEQ ID NO: 771 - DNA, 19 bp, Artificial Sequence
  Chemically Synthesized
gccgtttcaaggcctcatc

SEQ ID NO: 772 - DNA, 20 bp, Artificial Sequence
  Chemically Synthesized
tgagctgttctggcccctta

SEQ ID NO: 773 - DNA, 21 bp, Artificial Sequence
  Chemically Synthesized
ggagcaagctgaaagccatct

SEQ ID NO: 774 - DNA, 21 bp, Artificial Sequence
  Chemically Synthesized
ggctggtaggctggagagttc

SEQ ID NO: 775 - DNA, 21 bp, Artificial Sequence
  Chemically Synthesized
ctgtccctgtggtggtttgtg

SEQ ID NO: 776 - DNA, 21 bp, Artificial Sequence
  Chemically Synthesized
cgtcgcagtatttgtgtgcaa

SEQ ID NO: 777 - DNA, 22 bp, Artificial Sequence
  Chemically Synthesized
gaggagacacggcagctattct

SEQ ID NO: 778 - DNA, 21 bp, Artificial Sequence
  Chemically Synthesized
tgaagtacagggacgccatct

SEQ ID NO: 779 - DNA, 20 bp, Artificial Sequence
  Chemically Synthesized
tggctgcaggagtactggaa

SEQ ID NO: 780 - DNA, 21 bp, Artificial Sequence
  Chemically Synthesized
cctgaagggctggtcttgaag

SEQ ID NO: 781 - DNA, 21 bp, Artificial Sequence
  Chemically Synthesized
caacaaagtgcatggcaacag

SEQ ID NO: 782 - DNA, 19 bp, Artificial Sequence
  Chemically Synthesized
gaattcccggatgaggctg

SEQ ID NO: 783 - DNA, 20 bp, Artificial Sequence
  Chemically Synthesized
atgggagccagtcctatgca

SEQ ID NO: 784 - DNA, 21 bp, Artificial Sequence
  Chemically Synthesized
ccttcacaaacttgcttcgct

SEQ ID NO: 785 - DNA, 20 bp, Artificial Sequence
  Chemically Synthesized
tctcagccactgagggcaat

SEQ ID NO: 786 - DNA, 21 bp, Artificial Sequence
  Chemically Synthesized
ggaagttaatggtgccgaagg

SEQ ID NO: 787 - DNA, 21 bp, Artificial Sequence
  Chemically Synthesized
ctgctggagtggaaccaactg

SEQ ID NO: 788 - DNA, 20 bp, Artificial Sequence
  Chemically Synthesized
agggccgtgaacatgacttc

SEQ ID NO: 789 - DNA, 20 bp, Artificial Sequence
  Chemically Synthesized
caaagcaacgacaccttcca

SEQ ID NO: 790 - DNA, 20 bp, Artificial Sequence
  Chemically Synthesized
gccacatgcgctaaggagaa

SEQ ID NO: 791 - DNA, 21 bp, Artificial Sequence
  Chemically Synthesized
ccctaccaggagacggagaag

SEQ ID NO: 792 - DNA, 21 bp, Artificial Sequence
  Chemically Synthesized
gccaaacatggtccagaagag

SEQ ID NO: 793 - DNA, 21 bp, Artificial Sequence
  Chemically Synthesized
gggcaggtgacgacttctatg

SEQ ID NO: 794 - DNA, 20 bp, Artificial Sequence
  Chemically Synthesized
aggatgatgggcgtgatgtc

SEQ ID NO: 795 - DNA, 20 bp, Artificial Sequence
  Chemically Synthesized
ccacgaggtccgctgtaact

SEQ ID NO: 796 - DNA, 19 bp, Artificial Sequence
  Chemically Synthesized
cgtgagtgcctgaggctgt

SEQ ID NO: 797 - DNA, 20 bp, Artificial Sequence
  Chemically Synthesized
ccttcgctcatcgccttatc

SEQ ID NO: 798 - DNA, 19 bp, Artificial Sequence
  Chemically Synthesized
ccttgagttcccagcccag

SEQ ID NO: 799 - DNA, 21 bp, Artificial Sequence
  Chemically Synthesized
gcagcagctcctctccatatg

SEQ ID NO: 800 - DNA, 20 bp, Artificial Sequence
  Chemically Synthesized
cgaggaccacgaggaatttc

SEQ ID NO: 801 - DNA, 20 bp, Artificial Sequence
  Chemically Synthesized
tcctggacggagatgctcat

SEQ ID NO: 802 - DNA, 20 bp, Artificial Sequence
  Chemically Synthesized
tcctcccagatctccttgca

SEQ ID NO: 803 - DNA, 20 bp, Artificial Sequence
  Chemically Synthesized
cagcactgcacattgccatt

SEQ ID NO: 804 - DNA, 21 bp, Artificial Sequence
  Chemically Synthesized
tccattctccaccaagagggt

SEQ ID NO: 805 - DNA, 20 bp, Artificial Sequence
  Chemically Synthesized
ggtcatccttcgagacctgc

SEQ ID NO: 806 - DNA, 20 bp, Artificial Sequence
  Chemically Synthesized
ggctacagcaaagccgaaaa

SEQ ID NO: 807 - DNA, 20 bp, Artificial Sequence
  Chemically Synthesized
ctgatgaggagttccgggaa

SEQ ID NO: 808 - DNA, 20 bp, Artificial Sequence
  Chemically Synthesized
cgtttcggccattgcttaag

SEQ ID NO: 809 - DNA, 19 bp, Artificial Sequence
  Chemically Synthesized
gttgcgaatatggcctggg

SEQ ID NO: 810 - DNA, 21 bp, Artificial Sequence
  Chemically Synthesized
tcgatacggattctgctcctg

SEQ ID NO: 811 - DNA, 20 bp, Artificial Sequence
  Chemically Synthesized
tgcagcccaacaaaaccttt

SEQ ID NO: 812 - DNA, 21 bp, Artificial Sequence
  Chemically Synthesized
gggcacaagttcaagggactt

(19) United States
(12) Patent Application Publication
(10) Pub. No.: US 2008/0146502 A1
(43) Pub. Date: Jun. 19, 2008

(54) Methods for modulating the sensation of satiety perception and agents useful for same

(75) Inventors: Gregory Royce Collier, Barwon Heads (AU); Kenneth Russell Walder, Ocean Grove (AU)

Correspondence Address: THE MCCALLUM LAW FIRM, P. C., 685 BRIGGS STREET, PO BOX 929, ERIE, CO, 80516, US

(73) Assignee: Autogen Research Pty Ltd., Brighton (AU)

(21) Appl. No.: 11/710,138

(22) Filed: Feb. 23, 2007

Related U.S. Application Data
(63) Continuation-in-part of application No. 10/588,576, filed on May 24, 2007.
(60) Provisional application No. 60/541,862, filed on Feb. 3, 2004.
(60) Provisional application No. 60/592,052, filed on Jul. 28, 2004.

(51) Int. Cl. A61K 38/17; G01N 33/567; A61K 31/655; A61K 31/28; G01N 33/53; C07K 14/705; A61K 31/4164

(52) U.S. Cl. 514/12; 514/396; 514/150; 514/492; 435/7.2; 435/325; 435/287.2; 530/350

(57) ABSTRACT

The invention relates generally to a method for modulating the sensation of satiety perception and to agents useful for same. Such agents are useful in modulating, controlling or otherwise affecting inter alia obesity, anorexia, weight maintenance, metabolic energy levels and/or inflammatory conditions in a subject. More particularly, the invention identifies molecules which interact with ion channels thereby modulating the perception of satiety. The invention contemplates, therefore, a method for modulating the perception of gastric distension by the administration of agents which control the activation or inhibition of mechanoreceptors. The invention further provides compositions comprising these agents and methods of treatment using same. In one embodiment, the invention permits modulation of perception of gastric distension by the administration of agents which control the activation of mechanoreceptors associated with mechanical stretch.

### CROSS-REFERENCE TO RELATED APPLICATIONS

The present application is a continuation in part of U.S. Ser. No. 10/588,576 filed Aug. 3, 2006, which is a U.S. national phase filing under 35 U.S.C. 371 of PCT application No. PCT/AU2005/000120, filed Feb. 1, 2005, which claims the benefit of U.S. Provisional Patent Application No. 60/541,862, filed Feb. 3, 2004 and U.S. Provisional Patent Application No. 60/592,052, filed Jul. 28, 2004, each of which are hereby incorporated by reference in their entirety.

### BACKGROUND OF THE INVENTION

1. Field of the Invention

The invention relates generally to methods for modulating the sensation of satiety perception and to agents useful for same. Such agents are useful in modulating, controlling or otherwise affecting inter alia obesity, anorexia, weight maintenance, metabolic energy levels and/or inflammatory conditions in a subject. More particularly, the present invention identifies molecules which interact with ion channels thereby modulating the perception of satiety. The invention contemplates, therefore, a method for modulating the perception of gastric distension by the administration of agents which control the activation or inhibition of mechanoreceptors. The invention further provides compositions comprising these agents and methods of treatment using same. In one embodiment, the invention permits modulation of perception of gastric distension by the administration of agents which control the activation of mechanoreceptors associated with mechanical stretch.

2. Description of the Prior Art

Reference to any prior art in this specification is not, and should not be taken as, an acknowledgment or any form of suggestion that this prior art forms part of the common general knowledge in any country.

Obesity is defined as a pathological excess of body fat and is the result of an imbalance between energy intake and energy expenditure for a sustained period of time. Obesity is the most common metabolic disease found in affluent societies. The prevalence of obesity in these affluent societies is alarmingly high, ranging from 10% to upwards of 50% in some sub-populations. Of particular concern is the fact that the prevalence of obesity appears to be rising consistently in affluent societies and is now increasing rapidly in less prosperous nations as they become more affluent and/or adopt cultural practices similar to those in more affluent countries. The escalating rates of obesity globally have resulted in the World Health Organisation declaring an obesity epidemic worldwide.

In Australia, an AusDiab study estimated that 7.5 million Australians (60%) aged 25 years and over were overweight or obese. Of these, 2.6 million (21%) were obese (BMI>30). Similarly, the prevalence of obesity in the U.S. increased substantially between 1991 and 1998, increasing from 12% to 18% in Americans during this period.

The high and increasing prevalence of obesity has serious health implications for both individuals and society as a whole. Obesity is a complex and heterogeneous disorder and has been identified as a key risk indicator of preventable morbidity and mortality. Obesity, for example, increases the risk of a number of other metabolic conditions including Type 2 diabetes and cardiovascular disease. Alongside obesity the prevalence of diabetes continues to increase rapidly. The AusDiab survey referred to above estimated that close to 1 million Australians aged 25 years and over have Type 2 diabetes. This represents approximately 7.5% of the population. In the U.S., the number of adults with diabetes increased by 49% between 1991 and 2000. It has been estimated that about 17 million people in the U.S. have Type 2 diabetes and an equal number are thought to be pre-diabetic. In Australia, the annual costs of obesity associated with diabetes and other disease conditions has been conservatively estimated to be AUS$810 million for 1992-93. The direct costs of diabetes and its complications in Australia in 1993-94 were estimated at $681 million, or 2.2% of total health system costs in that year.

Obesity also has both metabolic and physiological bases and these need to be understood in development of therapeutic and prophylactic regimes. There is a need, therefore to investigate other physiological influences of obesity and other conditions.

The role of physiological gastric distension in the control of short-term satiety has been known for some time, and is mediated by both peptides secreted from the gastrointestinal tract in response to distension, as well as the enteric nervous system. Studies in rats have demonstrated that vagal afferent fibres respond to gastric distension, and mediate part of the physiological response. Further investigations demonstrated that most of the vagal nerve endings in the stomach that mediate the detection of distension have unique structural characteristics, and are known as Intraganglionic Laminar Endings (IGLEs) and Intramuscular Arrays (IMAs). These vagal nerve endings are responsive to gastric distension and play a role in short term satiety. However, the molecular mechanisms by which the physical stimulus of distension is converted into a chemical/electrical signal (a process known as mechanotransduction), and subsequent physiological adaptation, are unclear.

In accordance with the invention, it has been surprisingly determined that modulators of calcium flux across the cell membrane are useful in the treatment, prevention or modulation or control of obesity, anorexia, satiation, weight maintenance, metabolic energy levels and inflammatory conditions.

### SUMMARY OF THE INVENTION

In one embodiment, the invention is directed to a method for modulating the perception of satiety in a subject, the method comprising: administering to the subject an effective amount of an agent which modulates the level or activity of TRPV2, such that increasing or decreasing the level of or activity of TRPV2 changes the perception of satiety in the subject.

In another embodiment, the invention is directed to a method for screening for an agent which modulates the levels or activity of TRPV2 in a subject, the method comprising: screening for agents which interact or associate with TRPV2 or a portion thereof comprising an amino acid sequence selected from SEQ ID NO:1 through SEQ ID NO:752.

In a further embodiment, the invention is directed to a fragment of TRPV2 selected from SEQ ID NO:1 through SEQ ID NO:752.

The invention further contemplates a method for the prophylaxis or treatment of a condition including a condition characterized in part by the presence of a symptom associated with a disorder or disease associated with obesity, anorexia, need for satiation, weight maintenance, metabolic energy levels or inflammation in a subject, the method comprising the administration of a therapeutic agent selected from a calcium uptake inhibitor or promoter, a blocker or promoter of TRPV2 calcium channels and a biological dye which inhibits or promotes calcium uptake.

A summary of the sequence identifiers used throughout the subject specification is provided in Table 1a.

TABLE 1a
Summary of Sequence Identifiers

| SEQUENCE ID NO: | DESCRIPTION |
|---|---|
| SEQ ID NO: 1 | Amino acid fragment of TRPV2 |
| SEQ ID NO: 2 | Amino acid fragment of TRPV2 |
| SEQ ID NO: 3 | Amino acid fragment of TRPV2 |
| SEQ ID NO: 4 | Amino acid fragment of TRPV2 |
| SEQ ID NO: 5 | Amino acid fragment of TRPV2 |
| SEQ ID NO: 6 | Amino acid fragment of TRPV2 |
| SEQ ID NO: 7 | Amino acid fragment of TRPV2 |
| SEQ ID NO: 8 | Amino acid fragment of TRPV2 |
| SEQ ID NO: 9 | Amino acid fragment of TRPV2 |
| SEQ ID NO: 10 | Amino acid fragment of TRPV2 |
| SEQ ID NO: 11 | Amino acid fragment of TRPV2 |
| SEQ ID NO: 12 | Amino acid fragment of TRPV2 |
| SEQ ID NO: 13 | Amino acid fragment of TRPV2 |
| SEQ ID NO: 14 | Amino acid fragment of TRPV2 |
| SEQ ID NO: 15 | Amino acid fragment of TRPV2 |
| SEQ ID NO: 16 | Amino acid fragment of TRPV2 |
| SEQ ID NO: 17 | Amino acid fragment of TRPV2 |
| SEQ ID NO: 18 | Amino acid fragment of TRPV2 |
| SEQ ID NO: 19 | Amino acid fragment of TRPV2 |
| SEQ ID NO: 20 | Amino acid fragment of TRPV2 |
| SEQ ID NO: 21 | Amino acid fragment of TRPV2 |
| SEQ ID NO: 22 | Amino acid fragment of TRPV2 |
| SEQ ID NO: 23 | Amino acid fragment of TRPV2 |
| SEQ ID NO: 24 | Amino acid fragment of TRPV2 |
| SEQ ID NO: 25 | Amino acid fragment of TRPV2 |
| SEQ ID NO: 26 | Amino acid fragment of TRPV2 |
| SEQ ID NO: 27 | Amino acid fragment of TRPV2 |
| SEQ ID NO: 28 | Amino acid fragment of TRPV2 |
| SEQ ID NO: 29 | Amino acid fragment of TRPV2 |
| SEQ ID NO: 30 | Amino acid fragment of TRPV2 |
| SEQ ID NO: 31 | Amino acid fragment of TRPV2 |
| SEQ ID NO: 32 | Amino acid fragment of TRPV2 |
| SEQ ID NO: 33 | Amino acid fragment of TRPV2 |
| SEQ ID NO: 34 | Amino acid fragment of TRPV2 |
| SEQ ID NO: 35 | Amino acid fragment of TRPV2 |
| SEQ ID NO: 36 | Amino acid fragment of TRPV2 |
| SEQ ID NO: 37 | Amino acid fragment of TRPV2 |
| SEQ ID NO: 38 | Amino acid fragment of TRPV2 |
| SEQ ID NO: 39 | Amino acid fragment of TRPV2 |
| SEQ ID NO: 40 | Amino acid fragment of TRPV2 |
| SEQ ID NO: 41 | Amino acid fragment of TRPV2 |
| SEQ ID NO: 42 | Amino acid fragment of TRPV2 |
| SEQ ID NO: 43 | Amino acid fragment of TRPV2 |
| SEQ ID NO: 44 | Amino acid fragment of TRPV2 |
| SEQ ID NO: 45 | Amino acid fragment of TRPV2 |
| SEQ ID NO: 46 | Amino acid fragment of TRPV2 |
| SEQ ID NO: 47 | Amino acid fragment of TRPV2 |
| SEQ ID NO: 48 | Amino acid fragment of TRPV2 |
| SEQ ID NO: 49 | Amino acid fragment of TRPV2 |
| SEQ ID NO: 50 | Amino acid fragment of TRPV2 |
| SEQ ID NO: 51 | Amino acid fragment of TRPV2 |
| SEQ ID NO: 52 | Amino acid fragment of TRPV2 |
| SEQ ID NO: 53 | Amino acid fragment of TRPV2 |
| SEQ ID NO: 54 | Amino acid fragment of TRPV2 |
| SEQ ID NO: 55 | Amino acid fragment of TRPV2 |
| SEQ ID NO: 56 | Amino acid fragment of TRPV2 |
| SEQ ID NO: 57 | Amino acid fragment of TRPV2 |
| SEQ ID NO: 58 | Amino acid fragment of TRPV2 |
| SEQ ID NO: 59 | Amino acid fragment of TRPV2 |
| SEQ ID NO: 60 | Amino acid fragment of TRPV2 |
| SEQ ID NO: 61 | Amino acid fragment of TRPV2 |
| SEQ ID NO: 62 | Amino acid fragment of TRPV2 |
| SEQ ID NO: 63 | Amino acid fragment of TRPV2 |
| SEQ ID NO: 64 | Amino acid fragment of TRPV2 |
| SEQ ID NO: 65 | Amino acid fragment of TRPV2 |
| SEQ ID NO: 66 | Amino acid fragment of TRPV2 |
| SEQ ID NO: 67 | Amino acid fragment of TRPV2 |
| SEQ ID NO: 68 | Amino acid fragment of TRPV2 |
| SEQ ID NO: 69 | Amino acid fragment of TRPV2 |
| SEQ ID NO: 70 | Amino acid fragment of TRPV2 |
| SEQ ID NO: 71 | Amino acid fragment of TRPV2 |
| SEQ ID NO: 72 | Amino acid fragment of TRPV2 |
| SEQ ID NO: 73 | Amino acid fragment of TRPV2 |
| SEQ ID NO: 74 | Amino acid fragment of TRPV2 |
| SEQ ID NO: 75 | Amino acid fragment of TRPV2 |
| SEQ ID NO: 76 | Amino acid fragment of TRPV2 |
| SEQ ID NO: 77 | Amino acid fragment of TRPV2 |
| SEQ ID NO: 78 | Amino acid fragment of TRPV2 |
| SEQ ID NO: 79 | Amino acid fragment of TRPV2 |
| SEQ ID NO: 80 | Amino acid fragment of TRPV2 |
| SEQ ID NO: 81 | Amino acid fragment of TRPV2 |
| SEQ ID NO: 82 | Amino acid fragment of TRPV2 |
| SEQ ID NO: 83 | Amino acid fragment of TRPV2 |
| SEQ ID NO: 84 | Amino acid fragment of TRPV2 |
| SEQ ID NO: 85 | Amino acid fragment of TRPV2 |
| SEQ ID NO: 86 | Amino acid fragment of TRPV2 |
| SEQ ID NO: 87 | Amino acid fragment of TRPV2 |
| SEQ ID NO: 88 | Amino acid fragment of TRPV2 |
| SEQ ID NO: 89 | Amino acid fragment of TRPV2 |
| SEQ ID NO: 90 | Amino acid fragment of TRPV2 |
| SEQ ID NO: 91 | Amino acid fragment of TRPV2 |
| SEQ ID NO: 92 | Amino acid fragment of TRPV2 |
| SEQ ID NO: 93 | Amino acid fragment of TRPV2 |
| SEQ ID NO: 94 | Amino acid fragment of TRPV2 |
| SEQ ID NO: 95 | Amino acid fragment of TRPV2 |
| SEQ ID NO: 96 | Amino acid fragment of TRPV2 |
| SEQ ID NO: 97 | Amino acid fragment of TRPV2 |
| SEQ ID NO: 98 | Amino acid fragment of TRPV2 |
| SEQ ID NO: 99 | Amino acid fragment of TRPV2 |
| SEQ ID NO: 100 | Amino acid fragment of TRPV2 |
| SEQ ID NO: 101 | Amino acid fragment of TRPV2 |
| SEQ ID NO: 102 | Amino acid fragment of TRPV2 |
| SEQ ID NO: 103 | Amino acid fragment of TRPV2 |
| SEQ ID NO: 104 | Amino acid fragment of TRPV2 |
| SEQ ID NO: 105 | Amino acid fragment of TRPV2 |
| SEQ ID NO: 106 | Amino acid fragment of TRPV2 |
| SEQ ID NO: 107 | Amino acid fragment of TRPV2 |
| SEQ ID NO: 108 | Amino acid fragment of TRPV2 |
| SEQ ID NO: 109 | Amino acid fragment of TRPV2 |
| SEQ ID NO: 110 | Amino acid fragment of TRPV2 |
| SEQ ID NO: 111 | Amino acid fragment of TRPV2 |
| SEQ ID NO: 112 | Amino acid fragment of TRPV2 |
| SEQ ID NO: 113 | Amino acid fragment of TRPV2 |
| SEQ ID NO: 114 | Amino acid fragment of TRPV2 |
| SEQ ID NO: 115 | Amino acid fragment of TRPV2 |
| SEQ ID NO: 116 | Amino acid fragment of TRPV2 |
| SEQ ID NO: 117 | Amino acid fragment of TRPV2 |
| SEQ ID NO: 118 | Amino acid fragment of TRPV2 |
| SEQ ID NO: 119 | Amino acid fragment of TRPV2 |
| SEQ ID NO: 120 | Amino acid fragment of TRPV2 |
| SEQ ID NO: 121 | Amino acid fragment of TRPV2 |
| SEQ ID NO: 122 | Amino acid fragment of TRPV2 |
| SEQ ID NO: 123 | Amino acid fragment of TRPV2 |
| SEQ ID NO: 124 | Amino acid fragment of TRPV2 |
| SEQ ID NO: 125 | Amino acid fragment of TRPV2 |
| SEQ ID NO: 126 | Amino acid fragment of TRPV2 |
| SEQ ID NO: 127 | Amino acid fragment of TRPV2 |
| SEQ ID NO: 128 | Amino acid fragment of TRPV2 |
| SEQ ID NO: 129 | Amino acid fragment of TRPV2 |
| SEQ ID NO: 130 | Amino acid fragment of TRPV2 |
| SEQ ID NO: 131 | Amino acid fragment of TRPV2 |
| SEQ ID NO: 132 | Amino acid fragment of TRPV2 |
| SEQ ID NO: 133 | Amino acid fragment of TRPV2 |
| SEQ ID NO: 134 | Amino acid fragment of TRPV2 |
| SEQ ID NO: 135 | Amino acid fragment of TRPV2 |
| SEQ ID NO: 136 | Amino acid fragment of TRPV2 |
| SEQ ID NO: 137 | Amino acid fragment of TRPV2 |
| SEQ ID NO: 138 | Amino acid fragment of TRPV2 |
| SEQ ID NO: 139 | Amino acid fragment of TRPV2 |
| SEQ ID NO: 140 | Amino acid fragment of TRPV2 |
| SEQ ID NO: 141 | Amino acid fragment of TRPV2 |
| SEQ ID NO: 142 | Amino acid fragment of TRPV2 |
| SEQ ID NO: 143 | Amino acid fragment of TRPV2 |
| SEQ ID NO: 144 | Amino acid fragment of TRPV2 |
| SEQ ID NO: 145 | Amino acid fragment of TRPV2 |
| SEQ ID NO: 146 | Amino acid fragment of TRPV2 |
| SEQ ID NO: 147 | Amino acid fragment of TRPV2 |
| SEQ ID NO: 148 | Amino acid fragment of TRPV2 |
| SEQ ID NO: 149 | Amino acid fragment of TRPV2 |
| SEQ ID NO: 150 | Amino acid fragment of TRPV2 |
| SEQ ID NO: 151 | Amino acid fragment of TRPV2 |
| SEQ ID NO: 152 | Amino acid fragment of TRPV2 |
| SEQ ID NO: 153 | Amino acid fragment of TRPV2 |
| SEQ ID NO: 154 | Amino acid fragment of TRPV2 |
| SEQ ID NO: 155 | Amino acid fragment of TRPV2 |
| SEQ ID NO: 156 | Amino acid fragment of TRPV2 |
| SEQ ID NO: 157 | Amino acid fragment of TRPV2 |
| SEQ ID NO: 158 | Amino acid fragment of TRPV2 |
| SEQ ID NO: 159 | Amino acid fragment of TRPV2 |
| SEQ ID NO: 160 | Amino acid fragment of TRPV2 |
| SEQ ID NO: 161 | Amino acid fragment of TRPV2 |
| SEQ ID NO: 162 | Amino acid fragment of TRPV2 |
| SEQ ID NO: 163 | Amino acid fragment of TRPV2 |
| SEQ ID NO: 164 | Amino acid fragment of TRPV2 |
| SEQ ID NO: 165 | Amino acid fragment of TRPV2 |
| SEQ ID NO: 166 | Amino acid fragment of TRPV2 |
| SEQ ID NO: 167 | Amino acid fragment of TRPV2 |
| SEQ ID NO: 168 | Amino acid fragment of TRPV2 |
| SEQ ID NO: 169 | Amino acid fragment of TRPV2 |
| SEQ ID NO: 170 | Amino acid fragment of TRPV2 |
| SEQ ID NO: 171 | Amino acid fragment of TRPV2 |
| SEQ ID NO: 172 | Amino acid fragment of TRPV2 |
| SEQ ID NO: 173 | Amino acid fragment of TRPV2 |
| SEQ ID NO: 174 | Amino acid fragment of TRPV2 |
| SEQ ID NO: 175 | Amino acid fragment of TRPV2 |
| SEQ ID NO: 176 | Amino acid fragment of TRPV2 |
| SEQ ID NO: 177 | Amino acid fragment of TRPV2 |
| SEQ ID NO: 178 | Amino acid fragment of TRPV2 |
| SEQ ID NO: 179 | Amino acid fragment of TRPV2 |
| SEQ ID NO: 180 | Amino acid fragment of TRPV2 |
| SEQ ID NO: 181 | Amino acid fragment of TRPV2 |
| SEQ ID NO: 182 | Amino acid fragment of TRPV2 |
| SEQ ID NO: 183 | Amino acid fragment of TRPV2 |
| SEQ ID NO: 184 | Amino acid fragment of TRPV2 |
| SEQ ID NO: 185 | Amino acid fragment of TRPV2 |
| SEQ ID NO: 186 | Amino acid fragment of TRPV2 |
| SEQ ID NO: 187 | Amino acid fragment of TRPV2 |
| SEQ ID NO: 188 | Amino acid fragment of TRPV2 |
| SEQ ID NO: 189 | Amino acid fragment of TRPV2 |
| SEQ ID NO: 190 | Amino acid fragment of TRPV2 |
| SEQ ID NO: 191 | Amino acid fragment of TRPV2 |
| SEQ ID NO: 192 | Amino acid fragment of TRPV2 |
| SEQ ID NO: 193 | Amino acid fragment of TRPV2 |
| SEQ ID NO: 194 | Amino acid fragment of TRPV2 |
| SEQ ID NO: 195 | Amino acid fragment of TRPV2 |
| SEQ ID NO: 196 | Amino acid fragment of TRPV2 |
| SEQ ID NO: 197 | Amino acid fragment of TRPV2 |
| SEQ ID NO: 198 | Amino acid fragment of TRPV2 |
| SEQ ID NO: 199 | Amino acid fragment of TRPV2 |
| SEQ ID NO: 200 | Amino acid fragment of TRPV2 |
| SEQ ID NO: 201 | Amino acid fragment of TRPV2 |
| SEQ ID NO: 202 | Amino acid fragment of TRPV2 |
| SEQ ID NO: 203 | Amino acid fragment of TRPV2 |
| SEQ ID NO: 204 | Amino acid fragment of TRPV2 |
| SEQ ID NO: 205 | Amino acid fragment of TRPV2 |
| SEQ ID NO: 206 | Amino acid fragment of TRPV2 |
| SEQ ID NO: 207 | Amino acid fragment of TRPV2 |
| SEQ ID NO: 208 | Amino acid fragment of TRPV2 |
| SEQ ID NO: 209 | Amino acid fragment of TRPV2 |
| SEQ ID NO: 210 | Amino acid fragment of TRPV2 |
| SEQ ID NO: 211 | Amino acid fragment of TRPV2 |
| SEQ ID NO: 212 | Amino acid fragment of TRPV2 |
| SEQ ID NO: 213 | Amino acid fragment of TRPV2 |
| SEQ ID NO: 214 | Amino acid fragment of TRPV2 |
| SEQ ID NO: 215 | Amino acid fragment of TRPV2 |
| SEQ ID NO: 216 | Amino acid fragment of TRPV2 |
| SEQ ID NO: 217 | Amino acid fragment of TRPV2 |
| SEQ ID NO: 218 | Amino acid fragment of TRPV2 |
| SEQ ID NO: 219 | Amino acid fragment of TRPV2 |
| SEQ ID NO: 220 | Amino acid fragment of TRPV2 |
| SEQ ID NO: 221 | Amino acid fragment of TRPV2 |
| SEQ ID NO: 222 | Amino acid fragment of TRPV2 |
| SEQ ID NO: 223 | Amino acid fragment of TRPV2 |
| SEQ ID NO: 224 | Amino acid fragment of TRPV2 |
| SEQ ID NO: 225 | Amino acid fragment of TRPV2 |
| SEQ ID NO: 226 | Amino acid fragment of TRPV2 |
| SEQ ID NO: 227 | Amino acid fragment of TRPV2 |
| SEQ ID NO: 228 | Amino acid fragment of TRPV2 |
| SEQ ID NO: 229 | Amino acid fragment of TRPV2 |
| SEQ ID NO: 230 | Amino acid fragment of TRPV2 |
| SEQ ID NO: 231 | Amino acid fragment of TRPV2 |
| SEQ ID NO: 232 | Amino acid fragment of TRPV2 |
| SEQ ID NO: 233 | Amino acid fragment of TRPV2 |
| SEQ ID NO: 234 | Amino acid fragment of TRPV2 |
| SEQ ID NO: 235 | Amino acid fragment of TRPV2 |
| SEQ ID NO: 236 | Amino acid fragment of TRPV2 |
| SEQ ID NO: 237 | Amino acid fragment of TRPV2 |
| SEQ ID NO: 238 | Amino acid fragment of TRPV2 |
| SEQ ID NO: 239 | Amino acid fragment of TRPV2 |
| SEQ ID NO: 240 | Amino acid fragment of TRPV2 |
| SEQ ID NO: 241 | Amino acid fragment of TRPV2 |
| SEQ ID NO: 242 | Amino acid fragment of TRPV2 |
| SEQ ID NO: 243 | Amino acid fragment of TRPV2 |
| SEQ ID NO: 244 | Amino acid fragment of TRPV2 |
| SEQ ID NO: 245 | Amino acid fragment of TRPV2 |
| SEQ ID NO: 246 | Amino acid fragment of TRPV2 |
| SEQ ID NO: 247 | Amino acid fragment of TRPV2 |
| SEQ ID NO: 248 | Amino acid fragment of TRPV2 |
| SEQ ID NO: 249 | Amino acid fragment of TRPV2 |
| SEQ ID NO: 250 | Amino acid fragment of TRPV2 |
| SEQ ID NO: 251 | Amino acid fragment of TRPV2 |
| SEQ ID NO: 252 | Amino acid fragment of TRPV2 |
| SEQ ID NO: 253 | Amino acid fragment of TRPV2 |
| SEQ ID NO: 254 | Amino acid fragment of TRPV2 |
| SEQ ID NO: 255 | Amino acid fragment of TRPV2 |
| SEQ ID NO: 256 | Amino acid fragment of TRPV2 |
| SEQ ID NO: 257 | Amino acid fragment of TRPV2 |
| SEQ ID NO: 258 | Amino acid fragment of TRPV2 |
| SEQ ID NO: 259 | Amino acid fragment of TRPV2 |
| SEQ ID NO: 260 | Amino acid fragment of TRPV2 |
| SEQ ID NO: 261 | Amino acid fragment of TRPV2 |
| SEQ ID NO: 262 | Amino acid fragment of TRPV2 |
| SEQ ID NO: 263 | Amino acid fragment of TRPV2 |
| SEQ ID NO: 264 | Amino acid fragment of TRPV2 |
| SEQ ID NO: 265 | Amino acid fragment of TRPV2 |
| SEQ ID NO: 266 | Amino acid fragment of TRPV2 |
| SEQ ID NO: 267 | Amino acid fragment of TRPV2 |
| SEQ ID NO: 268 | Amino acid fragment of TRPV2 |
| SEQ ID NO: 269 | Amino acid fragment of TRPV2 |
| SEQ ID NO: 270 | Amino acid fragment of TRPV2 |
| SEQ ID NO: 271 | Amino acid fragment of TRPV2 |
| SEQ ID NO: 272 | Amino acid fragment of TRPV2 |
| SEQ ID NO: 273 | Amino acid fragment of TRPV2 |
| SEQ ID NO: 274 | Amino acid fragment of TRPV2 |
| SEQ ID NO: 275 | Amino acid fragment of TRPV2 |
| SEQ ID NO: 276 | Amino acid fragment of TRPV2 |
| SEQ ID NO: 277 | Amino acid fragment of TRPV2 |
| SEQ ID NO: 278 | Amino acid fragment of TRPV2 |
| SEQ ID NO: 279 | Amino acid fragment of TRPV2 |
| SEQ ID NO: 280 | Amino acid fragment of TRPV2 |
| SEQ ID NO: 281 | Amino acid fragment of TRPV2 |
| SEQ ID NO: 282 | Amino acid fragment of TRPV2 |
| SEQ ID NO: 283 | Amino acid fragment of TRPV2 |
| SEQ ID NO: 284 | Amino acid fragment of TRPV2 |
| SEQ ID NO: 285 | Amino acid fragment of TRPV2 |
| SEQ ID NO: 286 | Amino acid fragment of TRPV2 |
| SEQ ID NO: 287 | Amino acid fragment of TRPV2 |
| SEQ ID NO: 288 | Amino acid fragment of TRPV2 |
| SEQ ID NO: 289 | Amino acid fragment of TRPV2 |
| SEQ ID NO: 290 | Amino acid fragment of TRPV2 |
| SEQ ID NO: 291 | Amino acid fragment of TRPV2 |
| SEQ ID NO: 292 | Amino acid fragment of TRPV2 |
| SEQ ID NO: 293 | Amino acid fragment of TRPV2 |
| SEQ ID NO: 294 | Amino acid fragment of TRPV2 |
| SEQ ID NO: 295 | Amino acid fragment of TRPV2 |
| SEQ ID NO: 296 | Amino acid fragment of TRPV2 |
| SEQ ID NO: 297 | Amino acid fragment of TRPV2 |
| SEQ ID NO: 298 | Amino acid fragment of TRPV2 |
| SEQ ID NO: 299 | Amino acid fragment of TRPV2 |
| SEQ ID NO: 300 | Amino acid fragment of TRPV2 |
| SEQ ID NO: 301 | Amino acid fragment of TRPV2 |
| SEQ ID NO: 302 | Amino acid fragment of TRPV2 |
| SEQ ID NO: 303 | Amino acid fragment of TRPV2 |
| SEQ ID NO: 304 | Amino acid fragment of TRPV2 |
| SEQ ID NO: 305 | Amino acid fragment of TRPV2 |
| SEQ ID NO: 306 | Amino acid fragment of TRPV2 |
| SEQ ID NO: 307 | Amino acid fragment of TRPV2 |
| SEQ ID NO: 308 | Amino acid fragment of TRPV2 |
| SEQ ID NO: 309 | Amino acid fragment of TRPV2 |
| SEQ ID NO: 310 | Amino acid fragment of TRPV2 |
| SEQ ID NO: 311 | Amino acid fragment of TRPV2 |
| SEQ ID NO: 312 | Amino acid fragment of TRPV2 |
| SEQ ID NO: 313 | Amino acid fragment of TRPV2 |
| SEQ ID NO: 314 | Amino acid fragment of TRPV2 |
| SEQ ID NO: 315 | Amino acid fragment of TRPV2 |
| SEQ ID NO: 316 | Amino acid fragment of TRPV2 |
| SEQ ID NO: 317 | Amino acid fragment of TRPV2 |
| SEQ ID NO: 318 | Amino acid fragment of TRPV2 |
| SEQ ID NO: 319 | Amino acid fragment of TRPV2 |
| SEQ ID NO: 320 | Amino acid fragment of TRPV2 |
| SEQ ID NO: 321 | Amino acid fragment of TRPV2 |
| SEQ ID NO: 322 | Amino acid fragment of TRPV2 |
| SEQ ID NO: 323 | Amino acid fragment of TRPV2 |
| SEQ ID NO: 324 | Amino acid fragment of TRPV2 |
| SEQ ID NO: 325 | Amino acid fragment of TRPV2 |
| SEQ ID NO: 326 | Amino acid fragment of TRPV2 |
| SEQ ID NO: 327 | Amino acid fragment of TRPV2 |
| SEQ ID NO: 328 | Amino acid fragment of TRPV2 |
| SEQ ID NO: 329 | Amino acid fragment of TRPV2 |
| SEQ ID NO: 330 | Amino acid fragment of TRPV2 |
| SEQ ID NO: 331 | Amino acid fragment of TRPV2 |
| SEQ ID NO: 332 | Amino acid fragment of TRPV2 |
| SEQ ID NO: 333 | Amino acid fragment of TRPV2 |
| SEQ ID NO: 334 | Amino acid fragment of TRPV2 |
| SEQ ID NO: 335 | Amino acid fragment of TRPV2 |
| SEQ ID NO: 336 | Amino acid fragment of TRPV2 |
| SEQ ID NO: 337 | Amino acid fragment of TRPV2 |
| SEQ ID NO: 338 | Amino acid fragment of TRPV2 |
| SEQ ID NO: 339 | Amino acid fragment of TRPV2 |
| SEQ ID NO: 340 | Amino acid fragment of TRPV2 |
| SEQ ID NO: 341 | Amino acid fragment of TRPV2 |
| SEQ ID NO: 342 | Amino acid fragment of TRPV2 |
| SEQ ID NO: 343 | Amino acid fragment of TRPV2 |
| SEQ ID NO: 344 | Amino acid fragment of TRPV2 |
| SEQ ID NO: 345 | Amino acid fragment of TRPV2 |
| SEQ ID NO: 346 | Amino acid fragment of TRPV2 |
| SEQ ID NO: 347 | Amino acid fragment of TRPV2 |
| SEQ ID NO: 348 | Amino acid fragment of TRPV2 |
| SEQ ID NO: 349 | Amino acid fragment of TRPV2 |
| SEQ ID NO: 350 | Amino acid fragment of TRPV2 |
| SEQ ID NO: 351 | Amino acid fragment of TRPV2 |
| SEQ ID NO: 352 | Amino acid fragment of TRPV2 |
| SEQ ID NO: 353 | Amino acid fragment of TRPV2 |
| SEQ ID NO: 354 | Amino acid fragment of TRPV2 |
| SEQ ID NO: 355 | Amino acid fragment of TRPV2 |
| SEQ ID NO: 356 | Amino acid fragment of TRPV2 |
| SEQ ID NO: 357 | Amino acid fragment of TRPV2 |
| SEQ ID NO: 358 | Amino acid fragment of TRPV2 |
| SEQ ID NO: 359 | Amino acid fragment of TRPV2 |
| SEQ ID NO: 360 | Amino acid fragment of TRPV2 |
| SEQ ID NO: 361 | Amino acid fragment of TRPV2 |
| SEQ ID NO: 362 | Amino acid fragment of TRPV2 |
| SEQ ID NO: 363 | Amino acid fragment of TRPV2 |
| SEQ ID NO: 364 | Amino acid fragment of TRPV2 |
| SEQ ID NO: 365 | Amino acid fragment of TRPV2 |
| SEQ ID NO: 366 | Amino acid fragment of TRPV2 |
| SEQ ID NO: 367 | Amino acid fragment of TRPV2 |
| SEQ ID NO: 368 | Amino acid fragment of TRPV2 |
| SEQ ID NO: 369 | Amino acid fragment of TRPV2 |
| SEQ ID NO: 370 | Amino acid fragment of TRPV2 |
| SEQ ID NO: 371 | Amino acid fragment of TRPV2 |
| SEQ ID NO: 372 | Amino acid fragment of TRPV2 |
| SEQ ID NO: 373 | Amino acid fragment of TRPV2 |
| SEQ ID NO: 374 | Amino acid fragment of TRPV2 |
| SEQ ID NO: 375 | Amino acid fragment of TRPV2 |
| SEQ ID NO: 376 | Amino acid fragment of TRPV2 |
| SEQ ID NO: 377 | Amino acid fragment of TRPV2 |
| SEQ ID NO: 378 | Amino acid fragment of TRPV2 |
| SEQ ID NO: 379 | Amino acid fragment of TRPV2 |
| SEQ ID NO: 380 | Amino acid fragment of TRPV2 |
| SEQ ID NO: 381 | Amino acid fragment of TRPV2 |
| SEQ ID NO: 382 | Amino acid fragment of TRPV2 |
| SEQ ID NO: 383 | Amino acid fragment of TRPV2 |
| SEQ ID NO: 384 | Amino acid fragment of TRPV2 |
| SEQ ID NO: 385 | Amino acid fragment of TRPV2 |
| SEQ ID NO: 386 | Amino acid fragment of TRPV2 |
| SEQ ID NO: 387 | Amino acid fragment of TRPV2 |
| SEQ ID NO: 388 | Amino acid fragment of TRPV2 |
| SEQ ID NO: 389 | Amino acid fragment of TRPV2 |
| SEQ ID NO: 390 | Amino acid fragment of TRPV2 |
| SEQ ID NO: 391 | Amino acid fragment of TRPV2 |
| SEQ ID NO: 392 | Amino acid fragment of TRPV2 |
| SEQ ID NO: 393 | Amino acid fragment of TRPV2 |
| SEQ ID NO: 394 | Amino acid fragment of TRPV2 |
| SEQ ID NO: 395 | Amino acid fragment of TRPV2 |
| SEQ ID NO: 396 | Amino acid fragment of TRPV2 |
| SEQ ID NO: 397 | Amino acid fragment of TRPV2 |
| SEQ ID NO: 398 | Amino acid fragment of TRPV2 |
| SEQ ID NO: 399 | Amino acid fragment of TRPV2 |
| SEQ ID NO: 400 | Amino acid fragment of TRPV2 |
| SEQ ID NO: 401 | Amino acid fragment of TRPV2 |
| SEQ ID NO: 402 | Amino acid fragment of TRPV2 |
| SEQ ID NO: 403 | Amino acid fragment of TRPV2 |
| SEQ ID NO: 404 | Amino acid fragment of TRPV2 |
| SEQ ID NO: 405 | Amino acid fragment of TRPV2 |
| SEQ ID NO: 406 | Amino acid fragment of TRPV2 |
| SEQ ID NO: 407 | Amino acid fragment of TRPV2 |
| SEQ ID NO: 408 | Amino acid fragment of TRPV2 |
| SEQ ID NO: 409 | Amino acid fragment of TRPV2 |
| SEQ ID NO: 410 | Amino acid fragment of TRPV2 |
| SEQ ID NO: 411 | Amino acid fragment of TRPV2 |
| SEQ ID NO: 412 | Amino acid fragment of TRPV2 |
| SEQ ID NO: 413 | Amino acid fragment of TRPV2 |
| SEQ ID NO: 414 | Amino acid fragment of TRPV2 |
| SEQ ID NO: 415 | Amino acid fragment of TRPV2 |
| SEQ ID NO: 416 | Amino acid fragment of TRPV2 |
| SEQ ID NO: 417 | Amino acid fragment of TRPV2 |
| SEQ ID NO: 418 | Amino acid fragment of TRPV2 |
| SEQ ID NO: 419 | Amino acid fragment of TRPV2 |
| SEQ ID NO: 420 | Amino acid fragment of TRPV2 |
| SEQ ID NO: 421 | Amino acid fragment of TRPV2 |
| SEQ ID NO: 422 | Amino acid fragment of TRPV2 |
| SEQ ID NO: 423 | Amino acid fragment of TRPV2 |
| SEQ ID NO: 424 | Amino acid fragment of TRPV2 |
| SEQ ID NO: 425 | Amino acid fragment of TRPV2 |
| SEQ ID NO: 426 | Amino acid fragment of TRPV2 |
| SEQ ID NO: 427 | Amino acid fragment of TRPV2 |
| SEQ ID NO: 428 | Amino acid fragment of TRPV2 |
| SEQ ID NO: 429 | Amino acid fragment of TRPV2 |
| SEQ ID NO: 430 | Amino acid fragment of TRPV2 |
| SEQ ID NO: 431 | Amino acid fragment of TRPV2 |
| SEQ ID NO: 432 | Amino acid fragment of TRPV2 |
| SEQ ID NO: 433 | Amino acid fragment of TRPV2 |
| SEQ ID NO: 434 | Amino acid fragment of TRPV2 |
| SEQ ID NO: 435 | Amino acid fragment of TRPV2 |
| SEQ ID NO: 436 | Amino acid fragment of TRPV2 |
| SEQ ID NO: 437 | Amino acid fragment of TRPV2 |
| SEQ ID NO: 438 | Amino acid fragment of TRPV2 |
| SEQ ID NO: 439 | Amino acid fragment of TRPV2 |
| SEQ ID NO: 440 | Amino acid fragment of TRPV2 |
| SEQ ID NO: 441 | Amino acid fragment of TRPV2 |
| SEQ ID NO: 442 | Amino acid fragment of TRPV2 |
| SEQ ID NO: 443 | Amino acid fragment of TRPV2 |
| SEQ ID NO: 444 | Amino acid fragment of TRPV2 |
| SEQ ID NO: 445 | Amino acid fragment of TRPV2 |
| SEQ ID NO: 446 | Amino acid fragment of TRPV2 |
| SEQ ID NO: 447 | Amino acid fragment of TRPV2 |
| SEQ ID NO: 448 | Amino acid fragment of TRPV2 |
| SEQ ID NO: 449 | Amino acid fragment of TRPV2 |
| SEQ ID NO: 450 | Amino acid fragment of TRPV2 |
| SEQ ID NO: 451 | Amino acid fragment of TRPV2 |
| SEQ ID NO: 452 | Amino acid fragment of TRPV2 |
| SEQ ID NO: 453 | Amino acid fragment of TRPV2 |
| SEQ ID NO: 454 | Amino acid fragment of TRPV2 |
| SEQ ID NO: 455 | Amino acid fragment of TRPV2 |
| SEQ ID NO: 456 | Amino acid fragment of TRPV2 |
| SEQ ID NO: 457 | Amino acid fragment of TRPV2 |
| SEQ ID NO: 458 | Amino acid fragment of TRPV2 |
| SEQ ID NO: 459 | Amino acid fragment of TRPV2 |
| SEQ ID NO: 460 | Amino acid fragment of TRPV2 |
| SEQ ID NO: 461 | Amino acid fragment of TRPV2 |
| SEQ ID NO: 462 | Amino acid fragment of TRPV2 |
| SEQ ID NO: 463 | Amino acid fragment of TRPV2 |
| SEQ ID NO: 464 | Amino acid fragment of TRPV2 |
| SEQ ID NO: 465 | Amino acid fragment of TRPV2 |
| SEQ ID NO: 466 | Amino acid fragment of TRPV2 |
| SEQ ID NO: 467 | Amino acid fragment of TRPV2 |
| SEQ ID NO: 468 | Amino acid fragment of TRPV2 |
| SEQ ID NO: 469 | Amino acid fragment of TRPV2 |
| SEQ ID NO: 470 | Amino acid fragment of TRPV2 |
| SEQ ID NO: 471 | Amino acid fragment of TRPV2 |
| SEQ ID NO: 472 | Amino acid fragment of TRPV2 |
| SEQ ID NO: 473 | Amino acid fragment of TRPV2 |
| SEQ ID NO: 474 | Amino acid fragment of TRPV2 |
| SEQ ID NO: 475 | Amino acid fragment of TRPV2 |
| SEQ ID NO: 476 | Amino acid fragment of TRPV2 |
| SEQ ID NO: 477 | Amino acid fragment of TRPV2 |
| SEQ ID NO: 478 | Amino acid fragment of TRPV2 |
| SEQ ID NO: 479 | Amino acid fragment of TRPV2 |
| SEQ ID NO: 480 | Amino acid fragment of TRPV2 |
| SEQ ID NO: 481 | Amino acid fragment of TRPV2 |
| SEQ ID NO: 482 | Amino acid fragment of TRPV2 |
| SEQ ID NO: 483 | Amino acid fragment of TRPV2 |
| SEQ ID NO: 484 | Amino acid fragment of TRPV2 |
| SEQ ID NO: 485 | Amino acid fragment of TRPV2 |
| SEQ ID NO: 486 | Amino acid fragment of TRPV2 |
| SEQ ID NO: 487 | Amino acid fragment of TRPV2 |
| SEQ ID NO: 488 | Amino acid fragment of TRPV2 |
| SEQ ID NO: 489 | Amino acid fragment of TRPV2 |
| SEQ ID NO: 490 | Amino acid fragment of TRPV2 |
| SEQ ID NO: 491 | Amino acid fragment of TRPV2 |
| SEQ ID NO: 492 | Amino acid fragment of TRPV2 |
| SEQ ID NO: 493 | Amino acid fragment of TRPV2 |
| SEQ ID NO: 494 | Amino acid fragment of TRPV2 |
| SEQ ID NO: 495 | Amino acid fragment of TRPV2 |
| SEQ ID NO: 496 | Amino acid fragment of TRPV2 |
| SEQ ID NO: 497 | Amino acid fragment of TRPV2 |
| SEQ ID NO: 498 | Amino acid fragment of TRPV2 |
| SEQ ID NO: 499 | Amino acid fragment of TRPV2 |
| SEQ ID NO: 500 | Amino acid fragment of TRPV2 |
| SEQ ID NO: 501 | Amino acid fragment of TRPV2 |
| SEQ ID NO: 502 | Amino acid fragment of TRPV2 |
| SEQ ID NO: 503 | Amino acid fragment of TRPV2 |
| SEQ ID NO: 504 | Amino acid fragment of TRPV2 |
| SEQ ID NO: 505 | Amino acid fragment of TRPV2 |
| SEQ ID NO: 506 | Amino acid fragment of TRPV2 |
| SEQ ID NO: 507 | Amino acid fragment of TRPV2 |
| SEQ ID NO: 508 | Amino acid fragment of TRPV2 |
| SEQ ID NO: 509 | Amino acid fragment of TRPV2 |
| SEQ ID NO: 510 | Amino acid fragment of TRPV2 |
| SEQ ID NO: 511 | Amino acid fragment of TRPV2 |
| SEQ ID NO: 512 | Amino acid fragment of TRPV2 |
| SEQ ID NO: 513 | Amino acid fragment of TRPV2 |
| SEQ ID NO: 514 | Amino acid fragment of TRPV2 |
| SEQ ID NO: 515 | Amino acid fragment of TRPV2 |
| SEQ ID NO: 516 | Amino acid fragment of TRPV2 |
| SEQ ID NO: 517 | Amino acid fragment of TRPV2 |
| SEQ ID NO: 518 | Amino acid fragment of TRPV2 |
| SEQ ID NO: 519 | Amino acid fragment of TRPV2 |
| SEQ ID NO: 520 | Amino acid fragment of TRPV2 |
| SEQ ID NO: 521 | Amino acid fragment of TRPV2 |
| SEQ ID NO: 522 | Amino acid fragment of TRPV2 |
| SEQ ID NO: 523 | Amino acid fragment of TRPV2 |
| SEQ ID NO: 524 | Amino acid fragment of TRPV2 |
| SEQ ID NO: 525 | Amino acid fragment of TRPV2 |
| SEQ ID NO: 526 | Amino acid fragment of TRPV2 |
| SEQ ID NO: 527 | Amino acid fragment of TRPV2 |
| SEQ ID NO: 528 | Amino acid fragment of TRPV2 |
| SEQ ID NO: 529 | Amino acid fragment of TRPV2 |
| SEQ ID NO: 530 | Amino acid fragment of TRPV2 |
| SEQ ID NO: 531 | Amino acid fragment of TRPV2 |
| SEQ ID NO: 532 | Amino acid fragment of TRPV2 |
| SEQ ID NO: 533 | Amino acid fragment of TRPV2 |
| SEQ ID NO: 534 | Amino acid fragment of TRPV2 |
| SEQ ID NO: 535 | Amino acid fragment of TRPV2 |
| SEQ ID NO: 536 | Amino acid fragment of TRPV2 |
| SEQ ID NO: 537 | Amino acid fragment of TRPV2 |
| SEQ ID NO: 538 | Amino acid fragment of TRPV2 |
| SEQ ID NO: 539 | Amino acid fragment of TRPV2 |
| SEQ ID NO: 540 | Amino acid fragment of TRPV2 |
| SEQ ID NO: 541 | Amino acid fragment of TRPV2 |
| SEQ ID NO: 542 | Amino acid fragment of TRPV2 |
| SEQ ID NO: 543 | Amino acid fragment of TRPV2 |
| SEQ ID NO: 544 | Amino acid fragment of TRPV2 |
| SEQ ID NO: 545 | Amino acid fragment of TRPV2 |
| SEQ ID NO: 546 | Amino acid fragment of TRPV2 |
| SEQ ID NO: 547 | Amino acid fragment of TRPV2 |
| SEQ ID NO: 548 | Amino acid fragment of TRPV2 |
| SEQ ID NO: 549 | Amino acid fragment of TRPV2 |
| SEQ ID NO: 550 | Amino acid fragment of TRPV2 |
| SEQ ID NO: 551 | Amino acid fragment of TRPV2 |
| SEQ ID NO: 552 | Amino acid fragment of TRPV2 |
| SEQ ID NO: 553 | Amino acid fragment of TRPV2 |
| SEQ ID NO: 554 | Amino acid fragment of TRPV2 |
| SEQ ID NO: 555 | Amino acid fragment of TRPV2 |
| SEQ ID NO: 556 | Amino acid fragment of TRPV2 |
| SEQ ID NO: 557 | Amino acid fragment of TRPV2 |
| SEQ ID NO: 558 | Amino acid fragment of TRPV2 |
| SEQ ID NO: 559 | Amino acid fragment of TRPV2 |
| SEQ ID NO: 560 | Amino acid fragment of TRPV2 |
| SEQ ID NO: 561 | Amino acid fragment of TRPV2 |
| SEQ ID NO: 562 | Amino acid fragment of TRPV2 |
| SEQ ID NO: 563 | Amino acid fragment of TRPV2 |
| SEQ ID NO: 564 | Amino acid fragment of TRPV2 |
| SEQ ID NO: 565 | Amino acid fragment of TRPV2 |
| SEQ ID NO: 566 | Amino acid fragment of TRPV2 |
| SEQ ID NO: 567 | Amino acid fragment of TRPV2 |
| SEQ ID NO: 568 | Amino acid fragment of TRPV2 |
| SEQ ID NO: 569 | Amino acid fragment of TRPV2 |
| SEQ ID NO: 570 | Amino acid fragment of TRPV2 |
| SEQ ID NO: 571 | Amino acid fragment of TRPV2 |
| SEQ ID NO: 572 | Amino acid fragment of TRPV2 |
| SEQ ID NO: 573 | Amino acid fragment of TRPV2 |
| SEQ ID NO: 574 | Amino acid fragment of TRPV2 |
| SEQ ID NO: 575 | Amino acid fragment of TRPV2 |
| SEQ ID NO: 576 | Amino acid fragment of TRPV2 |
| SEQ ID NO: 577 | Amino acid fragment of TRPV2 |
| SEQ ID NO: 578 | Amino acid fragment of TRPV2 |
| SEQ ID NO: 579 | Amino acid fragment of TRPV2 |
| SEQ ID NO: 580 | Amino acid fragment of TRPV2 |
| SEQ ID NO: 581 | Amino acid fragment of TRPV2 |
| SEQ ID NO: 582 | Amino acid fragment of TRPV2 |
| SEQ ID NO: 583 | Amino acid fragment of TRPV2 |
| SEQ ID NO: 584 | Amino acid fragment of TRPV2 |
| SEQ ID NO: 585 | Amino acid fragment of TRPV2 |
| SEQ ID NO: 586 | Amino acid fragment of TRPV2 |
| SEQ ID NO: 587 | Amino acid fragment of TRPV2 |
| SEQ ID NO: 588 | Amino acid fragment of TRPV2 |
| SEQ ID NO: 589 | Amino acid fragment of TRPV2 |
| SEQ ID NO: 590 | Amino acid fragment of TRPV2 |
| SEQ ID NO: 591 | Amino acid fragment of TRPV2 |
| SEQ ID NO: 592 | Amino acid fragment of TRPV2 |
| SEQ ID NO: 593 | Amino acid fragment of TRPV2 |
| SEQ ID NO: 594 | Amino acid fragment of TRPV2 |
| SEQ ID NO: 595 | Amino acid fragment of TRPV2 |
| SEQ ID NO: 596 | Amino acid fragment of TRPV2 |
| SEQ ID NO: 597 | Amino acid fragment of TRPV2 |
| SEQ ID NO: 598 | Amino acid fragment of TRPV2 |
| SEQ ID NO: 599 | Amino acid fragment of TRPV2 |
| SEQ ID NO: 600 | Amino acid fragment of TRPV2 |
| SEQ ID NO: 601 | Amino acid fragment of TRPV2 |
| SEQ ID NO: 602 | Amino acid fragment of TRPV2 |
| SEQ ID NO: 603 | Amino acid fragment of TRPV2 |
| SEQ ID NO: 604 | Amino acid fragment of TRPV2 |
| SEQ ID NO: 605 | Amino acid fragment of TRPV2 |
| SEQ ID NO: 606 | Amino acid fragment of TRPV2 |
| SEQ ID NO: 607 | Amino acid fragment of TRPV2 |
| SEQ ID NO: 608 | Amino acid fragment of TRPV2 |
| SEQ ID NO: 609 | Amino acid fragment of TRPV2 |
| SEQ ID NO: 610 | Amino acid fragment of TRPV2 |
| SEQ ID NO: 611 | Amino acid fragment of TRPV2 |
| SEQ ID NO: 612 | Amino acid fragment of TRPV2 |
| SEQ ID NO: 613 | Amino acid fragment of TRPV2 |
| SEQ ID NO: 614 | Amino acid fragment of TRPV2 |
| SEQ ID NO: 615 | Amino acid fragment of TRPV2 |
| SEQ ID NO: 616 | Amino acid fragment of TRPV2 |
| SEQ ID NO: 617 | Amino acid fragment of TRPV2 |
| SEQ ID NO: 618 | Amino acid fragment of TRPV2 |
| SEQ ID NO: 619 | Amino acid fragment of TRPV2 |
| SEQ ID NO: 620 | Amino acid fragment of TRPV2 |
| SEQ ID NO: 621 | Amino acid fragment of TRPV2 |
| SEQ ID NO: 622 | Amino acid fragment of TRPV2 |
| SEQ ID NO: 623 | Amino acid fragment of TRPV2 |
| SEQ ID NO: 624 | Amino acid fragment of TRPV2 |
| SEQ ID NO: 625 | Amino acid fragment of TRPV2 |
| SEQ ID NO: 626 | Amino acid fragment of TRPV2 |
| SEQ ID NO: 627 | Amino acid fragment of TRPV2 |
| SEQ ID NO: 628 | Amino acid fragment of TRPV2 |
| SEQ ID NO: 629 | Amino acid fragment of TRPV2 |
| SEQ ID NO: 630 | Amino acid fragment of TRPV2 |
| SEQ ID NO: 631 | Amino acid fragment of TRPV2 |
| SEQ ID NO: 632 | Amino acid fragment of TRPV2 |
| SEQ ID NO: 633 | Amino acid fragment of TRPV2 |
| SEQ ID NO: 634 | Amino acid fragment of TRPV2 |
| SEQ ID NO: 635 | Amino acid fragment of TRPV2 |
| SEQ ID NO: 636 | Amino acid fragment of TRPV2 |
| SEQ ID NO: 637 | Amino acid fragment of TRPV2 |
| SEQ ID NO: 638 | Amino acid fragment of TRPV2 |
| SEQ ID NO: 639 | Amino acid fragment of TRPV2 |
| SEQ ID NO: 640 | Amino acid fragment of TRPV2 |
| SEQ ID NO: 641 | Amino acid fragment of TRPV2 |
| SEQ ID NO: 642 | Amino acid fragment of TRPV2 |
| SEQ ID NO: 643 | Amino acid fragment of TRPV2 |
| SEQ ID NO: 645 | Amino acid fragment of TRPV2 |
| SEQ ID NO: 646 | Amino acid fragment of TRPV2 |
| SEQ ID NO: 647 | Amino acid fragment of TRPV2 |
| SEQ ID NO: 648 | Amino acid fragment of TRPV2 |
| SEQ ID NO: 649 | Amino acid fragment of TRPV2 |
| SEQ ID NO: 650 | Amino acid fragment of TRPV2 |
| SEQ ID NO: 651 | Amino acid fragment of TRPV2 |
| SEQ ID NO: 652 | Amino acid fragment of TRPV2 |
| SEQ ID NO: 653 | Amino acid fragment of TRPV2 |
| SEQ ID NO: 654 | Amino acid fragment of TRPV2 |
| SEQ ID NO: 655 | Amino acid fragment of TRPV2 |
| SEQ ID NO: 656 | Amino acid fragment of TRPV2 |
| SEQ ID NO: 657 | Amino acid fragment of TRPV2 |
| SEQ ID NO: 658 | Amino acid fragment of TRPV2 |
| SEQ ID NO: 659 | Amino acid fragment of TRPV2 |
| SEQ ID NO: 660 | Amino acid fragment of TRPV2 |
| SEQ ID NO: 661 | Amino acid fragment of TRPV2 |
| SEQ ID NO: 662 | Amino acid fragment of TRPV2 |
| SEQ ID NO: 663 | Amino acid fragment of TRPV2 |
| SEQ ID NO: 664 | Amino acid fragment of TRPV2 |
| SEQ ID NO: 665 | Amino acid fragment of TRPV2 |
| SEQ ID NO: 666 | Amino acid fragment of TRPV2 |
| SEQ ID NO: 667 | Amino acid fragment of TRPV2 |
| SEQ ID NO: 668 | Amino acid fragment of TRPV2 |
| SEQ ID NO: 669 | Amino acid fragment of TRPV2 |
| SEQ ID NO: 670 | Amino acid fragment of TRPV2 |
| SEQ ID NO: 671 | Amino acid fragment of TRPV2 |
| SEQ ID NO: 672 | Amino acid fragment of TRPV2 |
| SEQ ID NO: 673 | Amino acid fragment of TRPV2 |
| SEQ ID NO: 674 | Amino acid fragment of TRPV2 |
| SEQ ID NO: 675 | Amino acid fragment of TRPV2 |
| SEQ ID NO: 676 | Amino acid fragment of TRPV2 |
| SEQ ID NO: 677 | Amino acid fragment of TRPV2 |
| SEQ ID NO: 678 | Amino acid fragment of TRPV2 |
| SEQ ID NO: 679 | Amino acid fragment of TRPV2 |
| SEQ ID NO: 680 | Amino acid fragment of TRPV2 |
| SEQ ID NO: 681 | Amino acid fragment of TRPV2 |
| SEQ ID NO: 682 | Amino acid fragment of TRPV2 |
| SEQ ID NO: 683 | Amino acid fragment of TRPV2 |
| SEQ ID NO: 684 | Amino acid fragment of TRPV2 |
| SEQ ID NO: 685 | Amino acid fragment of TRPV2 |
| SEQ ID NO: 686 | Amino acid fragment of TRPV2 |
| SEQ ID NO: 687 | Amino acid fragment of TRPV2 |
| SEQ ID NO: 688 | Amino acid fragment of TRPV2 |
| SEQ ID NO: 689 | Amino acid fragment of TRPV2 |
| SEQ ID NO: 690 | Amino acid fragment of TRPV2 |
| SEQ ID NO: 691 | Amino acid fragment of TRPV2 |
| SEQ ID NO: 692 | Amino acid fragment of TRPV2 |
| SEQ ID NO: 693 | Amino acid fragment of TRPV2 |
| SEQ ID NO: 694 | Amino acid fragment of TRPV2 |
| SEQ ID NO: 695 | Amino acid fragment of TRPV2 |
| SEQ ID NO: 696 | Amino acid fragment of TRPV2 |
| SEQ ID NO: 697 | Amino acid fragment of TRPV2 |
| SEQ ID NO: 698 | Amino acid fragment of TRPV2 |
| SEQ ID NO: 699 | Amino acid fragment of TRPV2 |
| SEQ ID NO: 700 | Amino acid fragment of TRPV2 |
| SEQ ID NO: 701 | Amino acid fragment of TRPV2 |
| SEQ ID NO: 702 | Amino acid fragment of TRPV2 |
| SEQ ID NO: 703 | Amino acid fragment of TRPV2 |
| SEQ ID NO: 704 | Amino acid fragment of TRPV2 |
| SEQ ID NO: 705 | Amino acid fragment of TRPV2 |
| SEQ ID NO: 706 | Amino acid fragment of TRPV2 |
| SEQ ID NO: 707 | Amino acid fragment of TRPV2 |
| SEQ ID NO: 708 | Amino acid fragment of TRPV2 |
| SEQ ID NO: 709 | Amino acid fragment of TRPV2 |
| SEQ ID NO: 710 | Amino acid fragment of TRPV2 |
| SEQ ID NO: 711 | Amino acid fragment of TRPV2 |
| SEQ ID NO: 712 | Amino acid fragment of TRPV2 |
| SEQ ID NO: 713 | Amino acid fragment of TRPV2 |
| SEQ ID NO: 714 | Amino acid fragment of TRPV2 |
| SEQ ID NO: 715 | Amino acid fragment of TRPV2 |
| SEQ ID NO: 716 | Amino acid fragment of TRPV2 |
| SEQ ID NO: 717 | Amino acid fragment of TRPV2 |
| SEQ ID NO: 718 | Amino acid fragment of TRPV2 |
| SEQ ID NO: 719 | Amino acid fragment of TRPV2 |
| SEQ ID NO: 720 | Amino acid fragment of TRPV2 |
| SEQ ID NO: 721 | Amino acid fragment of TRPV2 |
| SEQ ID NO: 722 | Amino acid fragment of TRPV2 |
| SEQ ID NO: 723 | Amino acid fragment of TRPV2 |
| SEQ ID NO: 724 | Amino acid fragment of TRPV2 |
| SEQ ID NO: 725 | Amino acid fragment of TRPV2 |
| SEQ ID NO: 726 | Amino acid fragment of TRPV2 |
| SEQ ID NO: 727 | Amino acid fragment of TRPV2 |
| SEQ ID NO: 728 | Amino acid fragment of TRPV2 |
| SEQ ID NO: 729 | Amino acid fragment of TRPV2 |
| SEQ ID NO: 730 | Amino acid fragment of TRPV2 |
| SEQ ID NO: 731 | Amino acid fragment of TRPV2 |
| SEQ ID NO: 732 | Amino acid fragment of TRPV2 |
| SEQ ID NO: 733 | Amino acid fragment of TRPV2 |
| SEQ ID NO: 734 | Amino acid fragment of TRPV2 |
| SEQ ID NO: 735 | Amino acid fragment of TRPV2 |
| SEQ ID NO: 736 | Amino acid fragment of TRPV2 |
| SEQ ID NO: 737 | Amino acid fragment of TRPV2 |
| SEQ ID NO: 738 | Amino acid fragment of TRPV2 |
| SEQ ID NO: 739 | Amino acid fragment of TRPV2 |
| SEQ ID NO: 740 | Amino acid fragment of TRPV2 |
| SEQ ID NO: 741 | Amino acid fragment of TRPV2 |
| SEQ ID NO: 742 | Amino acid fragment of TRPV2 |
| SEQ ID NO: 743 | Amino acid fragment of TRPV2 |
| SEQ ID NO: 744 | Amino acid fragment of TRPV2 |
| SEQ ID NO: 745 | Amino acid fragment of TRPV2 |
| SEQ ID NO: 746 | Amino acid fragment of TRPV2 |
| SEQ ID NO: 747 | Amino acid fragment of TRPV2 |
| SEQ ID NO: 748 | Amino acid fragment of TRPV2 |
| SEQ ID NO: 749 | Amino acid fragment of TRPV2 |
| SEQ ID NO: 750 | Amino acid fragment of TRPV2 |
| SEQ ID NO: 751 | Amino acid fragment of TRPV2 |
| SEQ ID NO: 752 | Amino acid sequence of TRPV2 |
| SEQ ID NO: 753 | Forward Primer SCNN1A |
| SEQ ID NO: 754 | Reverse Primer SCNN1A |
| SEQ ID NO: 755 | Forward Primer SCNN1B |
| SEQ ID NO: 756 | Reverse Primer SCNN1B |
| SEQ ID NO: 757 | Forward Primer SCNN1C |
| SEQ ID NO: 758 | Reverse Primer SCNN1C |
| SEQ ID NO: 759 | Forward Primer ACCN2 |
| SEQ ID NO: 760 | Reverse Primer ACCN2 |
| SEQ ID NO: 761 | Forward Primer ACCN1 |
| SEQ ID NO: 762 | Reverse Primer ACCN1 |
| SEQ ID NO: 763 | Forward Primer ACCN3 |
| SEQ ID NO: 764 | Reverse Primer ACCN3 |
| SEQ ID NO: 765 | Forward Primer ACCN4 |
| SEQ ID NO: 766 | Reverse Primer ACCN4 |
| SEQ ID NO: 767 | Forward Primer ACCN5 |
| SEQ ID NO: 768 | Reverse Primer ACCN5 |
| SEQ ID NO: 769 | Forward Primer KCNK2 |
| SEQ ID NO: 770 | Reverse Primer KCNK2 |
| SEQ ID NO: 771 | Forward Primer KCNK10 |
| SEQ ID NO: 772 | Reverse Primer KCNK10 |
| SEQ ID NO: 773 | Forward Primer KCNK4 |
| SEQ ID NO: 774 | Reverse Primer KCNK4 |
| SEQ ID NO: 775 | Forward Primer TRPM1 |
| SEQ ID NO: 776 | Reverse Primer TRPM1 |
| SEQ ID NO: 777 | Forward Primer TRPM2 |
| SEQ ID NO: 778 | Reverse Primer TRPM2 |
| SEQ ID NO: 779 | Forward Primer TRPM3 |
| SEQ ID NO: 780 | Reverse Primer TRPM3 |
| SEQ ID NO: 781 | Forward Primer TRPM4 |
| SEQ ID NO: 782 | Reverse Primer TRPM4 |
| SEQ ID NO: 783 | Forward Primer TRPM5 |
| SEQ ID NO: 784 | Reverse Primer TRPM5 |
| SEQ ID NO: 785 | Forward Primer TRPM6 |
| SEQ ID NO: 786 | Reverse Primer TRPM6 |
| SEQ ID NO: 787 | Forward Primer TRPM8 |
| SEQ ID NO: 788 | Reverse Primer TRPM8 |
| SEQ ID NO: 789 | Forward Primer TRPC1 |
| SEQ ID NO: 790 | Reverse Primer TRPC1 |
| SEQ ID NO: 791 | Forward Primer TRPC2 |
| SEQ ID NO: 792 | Reverse Primer TRPC2 |
| SEQ ID NO: 793 | Forward Primer TRPC3 |
| SEQ ID NO: 794 | Reverse Primer TRPC3 |
| SEQ ID NO: 795 | Forward Primer TRPC4 |
| SEQ ID NO: 796 | Reverse Primer TRPC4 |
| SEQ ID NO: 797 | Forward Primer TRPC5 |
| SEQ ID NO: 798 | Reverse Primer TRPC5 |
| SEQ ID NO: 799 | Forward Primer TRPC6 |
| SEQ ID NO: 800 | Reverse Primer TRPC6 |
| SEQ ID NO: 801 | Forward Primer TRPC7 |
| SEQ ID NO: 802 | Reverse Primer TRPC7 |
| SEQ ID NO: 803 | Forward Primer TRPV1 |
| SEQ ID NO: 804 | Reverse Primer TRPV1 |
| SEQ ID NO: 805 | Forward Primer TRPV2 |
| SEQ ID NO: 806 | Reverse Primer TRPV2 |
| SEQ ID NO: 807 | Forward Primer TRPV4 |
| SEQ ID NO: 808 | Reverse Primer TRPV4 |
| SEQ ID NO: 809 | Forward Primer TRPV5 |
| SEQ ID NO: 810 | Reverse Primer TRPV5 |
| SEQ ID NO: 811 | Forward Primer TRPV6 |
| SEQ ID NO: 812 | Reverse Primer TRPV6 |

### BRIEF DESCRIPTION OF THE FIGURE

FIG. 1 illustrates a representation of the amino acid sequence of TRPV2 in single letter code according to an embodiment of the present invention. Single and three letter codes for amino acids are provided in Table 1b.

The following single and three letter abbreviations for amino acid residues used in the specification are defined in Table 1b:

TABLE 1b
Amino Acid Abbreviations

| Amino Acid | Three-letter Abbreviation | One-letter Symbol |
|---|---|---|
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalamine | Phe | F |
| Proline | Pro | P |
| Serine | Ser | S |
| Threonine | Thr | T |
| Tryptophan | Trp | W |
| Tyrosine | Tyr | Y |
| Valine | Val | V |

### DETAILED DESCRIPTION OF THE INVENTION

The terminology used herein to describe the subject invention is for the purpose of describing particular embodiments and is not necessarily intended to be limiting.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

It should be noted that, as used in the subject specification, the singular forms “a”, “an” and “the” include plural aspects unless the context clearly dictates otherwise. Thus, for example, reference to “an agent” includes a single agent, as well as two or more agents; reference to “a mechanoreceptor” includes a single mechanoreceptor, as well as two or more mechanoreceptors; and so forth.

The invention is predicated, in part, upon the identification of specific molecules which interact with mechanoreceptors independent of mechanical stimulus or their functional, structural or evolutionary equivalents or homologues, including polymorphic variants which modulate the perception of satiety. Such molecules are important in modulating the sensation of satiety perception of a subject. Specifically, mechanoreceptors are identified which are involved in gastric distension. In accordance with one embodiment of the invention, agents which modulate the activity of these receptors are proposed to modulate a subject's perception of satiety. These agents enhance or suppress the function of the mechanoreceptors, including the activation of these mechanoreceptors, and alter the physical sensations associated with these receptors.

Reference to a “mechanoreceptor” encompasses any or all receptors involved in mechanotransduction including functional, structural or evolutionary equivalents or homologs including polymorphic variants, from any particular species. Human forms of mechanoreceptors are especially contemplated for use in developing agents for use in human subjects. However, the invention also has application in the veterinary, agricultural and wild life animal industries.

As used herein, the term “mechanoreceptor” refers to a specialised sensory end organ that responds to mechanical stimuli such as tension, pressure or displacement. Types of mechanoreceptors include ion channels such as EnaCs, ASICs, mechanosensitive potassium channels, transient receptor potential ion channels, muscle spindles and tendon organs. Reference to mechanoreceptors encompasses any or all ion channels which are involved in the process of mechanotransduction. In one embodiment, the mechanoreceptor includes calcium ion channels, including but not limited to, TRPV1 through TRPV6 inclusive and in one embodiment TRPV2.

In one embodiment, the ion channels include at least one of ENAC, PENAC, γENAC, ACCN3, ACCN4, ASIC 1, ASIC2, ASIC3, ASIC4, BLINAC/hiNaC (ACCN5), TREK1, TREK2, TRAAK (KCNK4), SCNN1C, KCNK2, TRPM1, TRPM2, TRPM3, TRPM4, TRPM6, TRPM7, TRPM8, TRPC1, TRPC2, TRPC3, TRPC4, TRPC6, TRPV1, TRPV2, TRPV3, TRPV6 and TRPM8. In addition, mechanoreceptors may also include homo- or hetero-multimeric complexes of one or more of these ion channels.

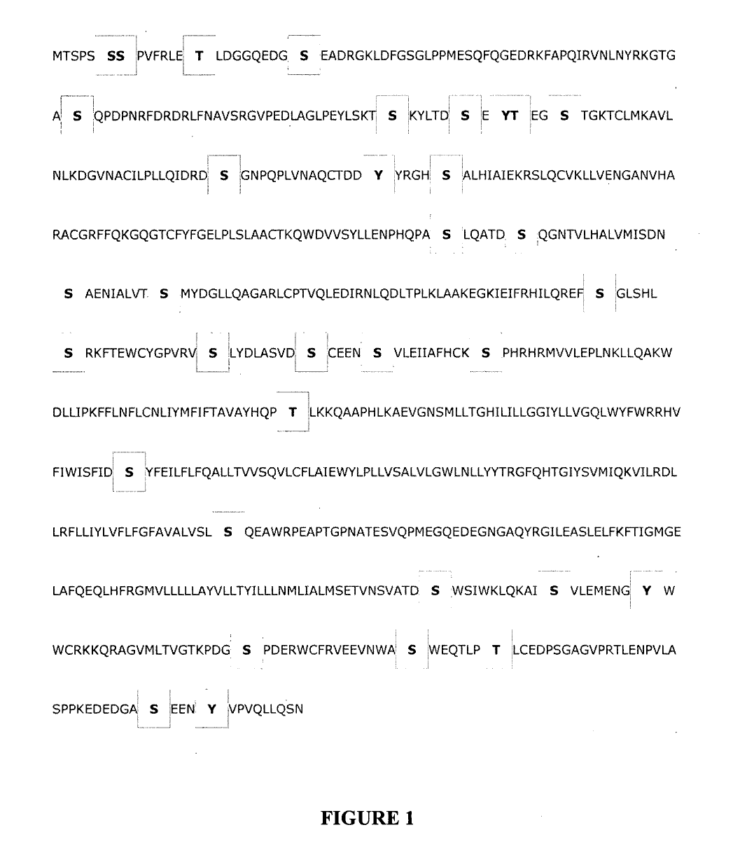

Reference to “TRPV2” includes its fragments or portions such as one or more of SEQ ID NO:1 though SEQ ID NO:752. In one embodiment, portions of TRPV2 including those exposed to an external environment when part of a cell membrane such as amino acid residue numbers 483 to 491 or 558 to 621 of the amino acid sequence of TRPV2 (SEQ ID NO:752), are utilized in the present invention. The amino acid sequence of TRPV2 is also represented in FIG. 1.

The invention extends to an isolated peptide or polypeptide including an amino acid sequence selected from SEQ ID NO:1 through SEQ ID NO:752, in particular when used as a target for an agent which interacts with and modulates the activity or levels of TRPV2.

Although the invention is particularly directed to the perception of satiety with regard to dietary requirements, homologous mechanoreceptors may be involved in perception of satiety in relation to other forms of physical stimuli and the modulation of those mechanoreceptors to mimic levels of satiety is also contemplated by the invention.

As used herein, reference to an “ion channel” includes any transmembrane protein or protein complex or non-proteinaceous component that forms or can be activated or induced to form a channel through which specific inorganic ions diffuse. Ion channel activation can occur via a membrane potential, drug, transmitter, cytoplasmic messenger, or a mechanical deformation or stretch. Activation or inactivation of an ion channel via a specific ligand can either occur via the ligand binding directly to a protein component of the ion channel (i.e. direct activation or inhibition) or may occur via the binding of a ligand of the mechanoreceptor, mechanoreceptor region or mechanoreceptor complex or a proteinaceous or non-proteinaceous component of the mechanoreceptor, mechanoreceptor region or mechanoreceptor complex (i.e. indirect activation or inhibition). Ion channels may be activated by both mechanical deformation or via the binding of a specific ligand resulting in mechanical deformation including conformational deformation or chemical or electrical stimulus. Ion channels of the invention are, in one embodiment, activated by a mechanical deformation.

Mechanoreceptors or ion channels of the invention are any mechanoreceptor or ion channel whose function, when altered, involves an alteration of the perception of satiety which would otherwise have occurred in response to a physical stimulus or a desire for a physical stimulus. In one aspect, the mechanoreceptors of the invention are those mechanoreceptors or ion channels associated with modulating the sensation of gastric distension. In one aspect, the mechanoreceptors or ion channels of the invention include one or more of ENAC, βENAC, γENAC, ACCN3, ACCN4, ASIC1, ASIC2, ASIC3, ASIC4, BLINAC/hiNaC (ACCN5), TREK1, TREK2, TRAAK (KCNK4), SCNN1C, KCNK2, TRPM1, TRPM2, TRPM3, TRPM4, TRPM6, TRPM7, TRPM8, TRPC1, TRPC2, TRPC3, TRPC4, TRPC6, TRPV1, TRPV2, TRPV3, TRPV6 and TRPM8 or a component or combination thereof.

Accordingly, one aspect of the invention contemplates a method for modulating the perception of satiety in a subject, said method including administering to said subject an effective amount of an agent comprising one or more of:
(i) an agent which is an agonist of a mechanoreceptor selected from the list consisting of ENAC, βENAC, γENAC, ACCN3, ACCN4, ASIC1, ASIC2, ASIC3, ASIC4, BLINAC/hiNaC (ACCN5), TREK1, TREK2, TRAAK (KCNK4), SCNN1C, KCNK2, TRPM1, TRPM2, TRPM3, TRPM4, TRPM6, TRPM7, TRPM8, TRPC1, TRPC2, TRPC3, TRPC4, TRPC6, TRPV1, TRPV2, TRPV3, TRPV6 and TRPM8;
(ii) an agent which is an antagonist of a mechanoreceptor list in (i);
(iii) an agent which inhibits expression of a gene encoding a mechanoreceptor listed in (i); and
(iv) an agent enhance expression of a gene encoding a mechanoreceptor listed in (i);
   wherein increasing or decreasing the level of or activity of the mechanoreceptors changes the perception of satiety in said subject.

In one embodiment, the invention provides a method for modulating the perception of satiety in a subject, the method including administering to the subject an effective amount of an agent which modulates the level or activity of TRPV2 wherein increasing or decreasing the level of or activity of TRPV2 changes the perception of satiety in the subject.

The phrase “modulating the sensation of satiety or perception of satiety” refers broadly to altering a subject's state of being satisfactorily full and not wanting to take more. This state may be in reference to any sense, and can involve any part of a subject. The phrase specifically encompasses altering the level of satiety to a specific sense in a subject. In one embodiment, sensation of satiety is associated with the gastric system and refers to a subject's dietary intake.

Reference to modulating the “sensation of satiety perception or perception of satiety” is meant in its broadest sense and encompasses agents which directly enhance or diminish the perception of satiety by either increasing or decreasing or blocking the activation of mechanoreceptors. Of the latter agents, genetic silencing agents such as to small interfering RNAs or ribozymes which reduce or inhibit ligand availability, dominant negative mutants or parts of the ligand which affect binding but not functional activity are contemplated, among others. The functional consequences of modulating mechanoreceptor activation using an effective amount of an agent is to alter the perception of a particular sensation without supplying mechanical pressure or distortion.

The term “effective amount” of an agent means a sufficient amount of the agent to provide the desired therapeutic or physiological effect. Thus, an “effective amount” of an agent includes a sufficient amount of the agent to modulate the sensation of satiety perception in a subject. Undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation.

Agents of the invention encompass compounds which selectively bind to a mechanoreceptor, either enhancing, preventing or diminishing the function of the mechanoreceptor, including its activation. The agents of the present invention may function in a variety of ways. These include, without being limited to, agents which cover or block the channel of the mechanoreceptor or ion channel preventing the transfer of ions across the cell membrane. In addition, these agents bind to a compartment at or proximal to the site of the mechanoreceptor which results in a conformational alteration to the membrane or the mechanoreceptor complex itself. Alternatively, or as well as, the agents may bind to a component of the mechanoreceptor or ion channel thereby influencing its ability to participate in the functioning of the mechanoreceptor complex. Components of the mechanoreceptor may be proteinaceous, non-proteinaceous, lipids or carbohydrates. Conversely, the agents of the invention may bind to a ligand separate from the mechanoreceptor or ion channel, and as such either enhance, prevent or diminish the function of the mechanoreceptor, including its activation, directly or indirectly.

Useful agents that interact or associate with a portion of TRPV2 thereby modulating levels or activities of TRPV2 may be used in the invention. Such portions include interaction with a portion of TRPV2 comprising one or more of SEQ ID NO:1 through SEQ ID NO:752.

In one embodiment, the agent interacts or associates with an externally exposed portion of TRPV2 such as amino acids 483 to 491 or 558 to 621 of SEQ ID NO:752.

One form of an agent useful in the practice of the present invention is derived from a molecule which interacts with the mechanoreceptor or component thereof and in particular a naturally occurring endogenous ligand. In one embodiment where the ligand is a proteinaceous or non-proteinaceous molecule, the agent may be a part or derivative of that ligand. A fragment of a proteinaceous ligand is contemplated includes from about 5 to at least about 30 contiguous amino acids. With respect to a non-proteinaceous, carbohydrate based ligand, such as a GAG or a polyunsaturated fatty acid, the agent may include a derivative of these molecules comprising up to less than about 10% the full length molecule.

For example, proteinaceous agents of this type may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of an amino acid sequence of the invention with proteinases such as endoLys-C, endoArg-C, endoGlu-C and staphylococcus V8-protease. The digested fragments can be purified by, for example, high performance liquid chromatographic (HPLC) techniques. Any such fragment, irrespective of its means of generation, is to be understood as being encompassed by the term “derivative” as used herein.

Thus, proteinaceous derivatives, or the singular proteinaceous derivative, encompass parts, mutants, homologs, fragments, analogues as well as hybrid or fusion molecules and glycosylation variants. Derivatives also include molecules having a percent amino acid sequence similarity over a window of comparison after optimal alignment with the naturally occurring molecule with a difference of at least 1%.

Another form of an agent comprises the naturally occurring molecule having a chemical modification. Such a molecule is referred to herein as an analog. Analogs contemplated herein include but are not limited to modification to side chains, incorporating of unnatural amino acids and/or their derivatives during peptide, polypeptide or protein synthesis and the use of crosslinkers and other methods which impose conformational constraints on the proteinaceous molecule or their analogs. This term also does not exclude modifications of the polypeptide, for example, glycosylations, acetylations, phosphorylations and the like. Included within the definition are, for example, polypeptides containing one or more analogs of an amino acid (including, for example, unnatural amino acids such as those given in Table 2) or polypeptides with substituted linkages. Such polypeptides may need to be able to enter the cell.

Examples of side chain modifications contemplated by the invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid, contemplated herein is shown in Table 2.

TABLE 2
Codes for non-conventional amino acids

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-Nmethylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N a methylamino acids, introduction of double bonds between C_{α} and C_{β} atoms of amino acids and the formation of cyclic peptides or analogs by introducing covalent bonds such as forming an amide bond between the N and C termini, between two side chains or between a side chain and the N or C terminus.

Mimetics are another useful group of compounds. The term is intended to refer to a substance which has some chemical similarity to the molecule it mimics, but which enhances or diminishes or prevents the activation of a mechanoreceptor. A peptide mimetic may be a peptide-containing molecule that mimics elements of protein secondary structure. The underlying rationale behind the use of peptide mimetics is that the peptide backbone of proteins exists chiefly to orient amino acid side chains in such a way as to facilitate molecular interactions such as those of antibody and antigen, enzyme and substrate or scaffolding proteins. A peptide mimetic is designed to permit molecular interactions similar to the natural molecule. Peptide or non-peptide mimetics may be useful, for example by up-regulating the activation of a mechanoreceptor and thereby increase the perception of satiety. Alternatively, the peptide or non-peptide mimetics may be useful in down-regulating or suppressing the activation of a mechanoreceptor, thereby decreasing the sensation of satiety perception.

The designing of mimetics to a pharmaceutically active compound is a known approach to the development of pharmaceuticals based on a “lead” compound. This might be desirable where the active compound is difficult or expensive to synthesize or where it is unsuitable for a particular method of administration, e.g. peptides are unsuitable active agents for oral compositions as they tend to be quickly degraded by proteases in the alimentary canal. Mimetic design, synthesis and testing is generally used to avoid randomly screening large numbers of molecules for a target property. In a particular embodiment, the mimetic is of TRPV2.

There are several steps commonly taken in the design of a mimetic from a compound having a given target property. First, the particular parts of the compound that are critical and/or important in determining the target property are determined. In the case of a peptide, this can be done by systematically varying the amino acid residues in the peptide, e.g. by substituting each residue in turn. Alanine scans of peptides are commonly used to refine such peptide motifs. These parts or residues constituting the active region of the compound are known as its “pharmacophore”.

Once the pharmacophore has been found, its structure is modelled according to its physical properties, e.g. stereochemistry, bonding, size and/or charge, using data from a range of sources, e.g. spectroscopic techniques, x-ray diffraction data and NMR. Computational analysis, similarity mapping (which models the charge and/or volume of a pharmacophore, rather than the bonding between atoms) and other techniques can be used in this modelling process.

In a variant of this approach, the three-dimensional structure of the receptor and its binding partner are modelled. This can be especially useful where the receptor and/or binding partner change conformation on binding, allowing the model to take account of this in the design of the mimetic. Modelling can be used to generate inhibitors which interact with the linear sequence or a three-dimensional configuration.

A template molecule is then selected onto which chemical groups which mimic the pharmacophore can be grafted. The template molecule and the chemical groups grafted onto it can conveniently be selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent they exhibit it. Further optimization or modification can then be carried out to arrive at one or more final mimetics for in vivo or clinical testing.

The goal of rational drug design is to produce structural analogs of biologically active polypeptides of interest or of small molecules with which they interact (e.g. agonists, antagonists, inhibitors or enhancers) in order to fashion drugs which are, for example, more active or stable forms of the polypeptide, or which, e.g. enhance or interfere with the function of a polypeptide in vivo. In one approach, one first determines the three-dimensional structure of a protein of interest by x-ray crystallography, by computer modelling or most typically, by a combination of approaches. Useful information regarding the structure of a polypeptide may also be gained by modelling based on the structure of homologous proteins. An example of rational drug design is the development of HIV protease inhibitors.

It is particularly useful to express all or part of the TRPV2 or other receptor on a cell surface or on a solid support and screen for molecules which interact with one or more portions. For example, TRPV2 portions include a peptide or polypeptide including an amino acid sequence selected from SEQ ID NO:1 through SEQ ID NO:752. In one embodiment, useful portions include externally exposed amino acids such as residues 483 to 491 or 558 to 621 of TRPV2.

Hence, the invention extends to an agent which interacts with TRPV2 thus modulating its activity or level, said agent interacting with a portion of TRPV2 including an amino acid sequence selected from SEQ ID NO:1 through SEQ ID NO:752.

Accordingly, the invention contemplates a method for screening for an agent which modulates the levels or activity of TRPV2 in a subject, said method comprising screening for agents which interact or associate with TRPV2 or a portion thereof comprising an amino acid sequence selected from SEQ ID NO:1 through SEQ ID NO:752.

In one embodiment, the agent interacts with one or both of regions defined by amino acid residue 483 to 491 and/or 558 to 621 of TRPV2.

The invention extends to a synthetic or recombinant molecule comprising first and second portions where at least one portion includes an amino acid sequence selected from the list consisting of SEQ ID NO:1 through SEQ ID NO:752 including amino acids 483 to 491 or 558 to 621 of TRPV2.

The other of the first or second portions may be a protein, polypeptide, peptide, solid support, resin, bead or the like.

Proteinaceous agents of the invention may be conveniently prepared by modifying the nucleotide sequence of a genetic molecule encoding a naturally occurring mechanoreceptor ligand. In relation to genetic molecules, the terms mutant, part, derivative, homolog, analog or mimetic have, mutatis mutandis, analogous meanings to the meanings ascribed to these forms in relation to proteinaceous molecules.

A nucleic acid encoding of a naturally occurring ligand is conveniently defined as the naturally occurring nucleotide sequence with a single or multiple nucleotide substitution, deletion or addition.

The terms “similarity” or “identity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particular embodiment, nucleotide and amino acid sequence comparisons are made at the level of identity rather than similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerised implementations of algorithms or by inspection and the best alignment (i.e. resulting in the highest percentage homology over the comparison window) generated by any of the various methods selected. Reference also may be made to the BLAST family of programs as, for example, disclosed by Altschul et al. (Nucl. Acids Res. 25: 3389, 1997). A detailed discussion of sequence analysis can be found in Unit 19.3 of Ausubel et al. (“Current Protocols in Molecular Biology” John Wiley & Sons Inc, 1994-1998, Chapter 15).

The terms “sequence similarity” and “sequence identity” as used herein refer to the extent that sequences are identical or functionally or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, H is, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

Derivatives of naturally occurring genetic molecules may also be defined as being encoded by a nucleic acid molecule which is capable of hybridizing to a reference sequence or a complementary form thereof under low stringency conditions.

Reference herein to a low stringency includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C) %. However, the Tₘ of a duplex nucleic acid molecule decreases by 1° C. with every increase of 1% in the number of mismatch base pairs. Formamide is optional in these hybridization conditions. Accordingly, in one embodiment, the levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

Agents useful in the practice of the invention may include chemical agents such as synthetic or recombinant molecules, polypeptides, peptides or proteins, lipids, glycoproteins or other naturally or non-naturally occurring molecules or analogs thereof. Alternatively, agents may include genetic agents such as nucleic acid molecules or modified forms thereof. Examples of such nucleic acid molecules include DNA (genomic, cDNA), RNA (sense RNAs, antisense RNAs, mRNAs, tRNAs, rRNAs, small interfering RNAs (SiRNAs), micro RNAs (miRNAs), small nucleolar RNAs (SnoRNAs), small nuclear (SnRNAs)) ribozymes, aptamers, DNAzymes or other ribonuclease-type complexes. Other nucleic acid molecules will include promoters or enhancers or other regulatory regions which modulate transcription. The aim of these molecules is to modulate the levels of components involved in the mechanoreceptor complex.

Accordingly, the invention extends to a genetic approach for modulating the perception of satiety using nucleic acid constructs which modulate the levels of proteinaceous components.

The terms “nucleic acids”, “nucleotide” and “polynucleotide” include RNA, cDNA, genomic DNA, synthetic forms and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog (such as the morpholine ring), internucleotide modifications such as uncharged linkages (e.g. methyl phosphonates, phosphotriesters, phosphoamidates, carbamates, etc.), charged linkages (e.g. phosphorothioates, phosphorodithioates, etc.), pendent moieties (e.g. polypeptides), intercalators (e.g. acridine, psoralen, etc.), chelators, alkylators and modified linkages (e.g. α-anomeric nucleic acids, etc.). Also included are synthetic molecules that mimic polynucleotides in their ability to bind to a designated sequence via hydrogen binding and other chemical interactions. Such molecules are known in the art and include, for example, those in which peptide linkages substitute for phosphate linkages in the backbone of the molecule.

Antisense polynucleotide sequences, for example, are useful in silencing transcripts. Furthermore, polynucleotide vectors containing all or a portion of a mechanoreceptor gene locus or molecule which binds to a mechanoreceptor may be placed under the control of a promoter in an antisense orientation and introduced into a cell. Expression of such an antisense construct within a cell will interfere with target transcription and/or translation. Furthermore, co-suppression and mechanisms to induce RNAi or siRNA may also be employed. Alternatively, antisense or sense molecules may be directly administered. In this latter embodiment, the antisense or sense molecules may be formulated in a composition and then administered by any number of means to target cells.

A variation on antisense and sense molecules involves the use of morpholinos, which are oligonucleotides composed of morpholine nucleotide derivatives and phosphorodiamidate linkages (for example, Summerton and Weller, Antisense and Nucleic Acid Drug Development 7: 187-195, 1997). Such compounds are injected into embryos and the effect of interference with mRNA is observed.

In one embodiment, the invention employs compounds such as oligonucleotides and similar species for use in modulating the activation of a mechanoreceptor. This is accomplished by providing oligonucleotides which specifically hybridize with one or more mechanoreceptors. The oligonucleotides may be provided directly to a cell or generated within the cell. As used herein, the terms “target nucleic acid” and “nucleic acid molecule encoding an inhibitor” have been used for convenience to encompass DNA encoding the inhibitor, RNA (including pre-mRNA and mRNA or portions thereof) transcribed from such DNA, and also cDNA derived from such RNA. The hybridization of a compound of the subject invention with its target nucleic acid is generally referred to as “antisense”. Consequently, the mechanism believed to be included in the practice of some embodiments of the invention is referred to herein as “antisense inhibition.” Such antisense inhibition is typically based upon hydrogen bonding-based hybridization of oligonucleotide strands or segments such that at least one strand or segment is cleaved, degraded, or otherwise rendered inoperable. In this regard, in a particular embodiment, it may be beneficial to target specific nucleic acid molecules and their functions for such antisense inhibition.

The functions of DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. In one example, the result of such interference with target nucleic acid function in modulation of the activation of a mechanoreceptor. In the context of the present invention, “modulation” and “modulation of activation” mean either an increase (enhanced) or a decrease (inhibition) in the activation of an mechanoreceptor.

An antisense compound is specifically hybridizable when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired, i.e. under physiological conditions in the case of in vivo assays or therapeutic treatment, and under conditions in which assays are performed in the case of in vitro assays.

“Complementary” as used herein, refers to the capacity for precise pairing between two nucleobases of an oligomeric compound. For example, if a nucleobase at a certain position of an oligonucleotide (an oligomeric compound), is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

According to the invention, compounds include antisense oligomeric compounds, antisense oligonucleotides, ribozymes, external guide sequence (EGS) oligonucleotides, alternate splicers, primers, probes, and other oligomeric compounds which hybridize to at least a portion of the target nucleic acid. As such, these compounds may be introduced in the form of single-stranded, double-stranded, circular or hairpin oligomeric compounds and may contain structural elements such as internal or terminal bulges or loops. Once introduced to a system, the compounds of the invention may elicit the action of one or more enzymes or structural proteins to effect modification of the target nucleic acid. One non-limiting example of such an enzyme is RNAse H, a cellular endonuclease which cleaves the RNA strand of an RNA:DNA duplex. It is known in the art that single-stranded antisense compounds which are “DNA-like” elicit RNAse H. Activation of RNase H, therefore, results in cleavage of the RNA target, thereby greatly enhancing the efficiency of oligonucleotide-mediated inhibition of gene expression. Similar roles have been postulated for other ribonucleases such as those in the RNase III and ribonuclease L family of enzymes.

While in one embodiment the form of antisense compound is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products. This phenomenon occurs in both plants and animals.

While oligonucleotides are a form of the compounds of this invention, the invention contemplates other families of compounds as well, including but not limited to oligonucleotides, analogs and mimetics such as those herein described.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound. In addition, linear compounds may have internal nucleobase complementarity and may, therefore, fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Specific examples of antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

The invention further extends to a nucleic acid (including a isolated nucleic acid) including a nucleotide sequence encoding an amino acid sequence selected which includes a sequence from SEQ ID NO:1 through SEQ ID NO:752 including amino acid residues 483 to 491 or 558 to 621 of TRPV2 (SEQ ID NO:753).

Such nucleic acid molecules may be useful for generating antisense molecules and/or generating fusion protein molecules for targets for agents.

In a further aspect of the invention, the agents interact with the mechanoreceptor to promote or disrupt activation of the mechanoreceptor and modulate satiety.

In another embodiment, the agent enhances activation of the mechanoreceptor and alters the perception of satiety.

In another embodiment, the agent decreases or prevents activation of the mechanoreceptor and alters the perception of satiety.

Reference to a cell herein includes any cell which expresses mechanoreceptors. The mechanoreceptors may either be endogenously produced or the cell may be genetically manipulated to produce mechanoreceptors. In some embodiments, the mechanoreceptors of the invention are related to responses associated with gastric distension.

In a further aspect of this embodiment, the agent essentially includes all or part of the sequence of amino acids forming the activation portion of a mechanoreceptor or a molecule which binds to the activation portion of a mechanoreceptor.

In a further aspect of the invention, the agents which modulate activation of a mechanoreceptor and/or enhance or decrease or prevent activation of a mechanoreceptor are agents which increase or decrease the perception of satiety.

In another embodiment, the invention provides a method for modulating the sensation of satiety perception in a subject comprising administering to the subject an agent which either increases or decreases or prevents activation of a mechanoreceptor.

In one aspect, the invention provides methods for enhancing the perception of satiety and thereby decreasing a subject's desire to eat. In a related aspect, the invention provides methods for suppressing the sensation of satiety perception and thereby increasing the desire of a subject to eat.

Screening assays for establishing the effects of different agents are well known to those of skill in the art and include such assays as FRET and FLIPR and patch-clamp and voltage-clamp, all of which screen the function of an ion channel.

Fluorescence resonance energy transfer (FRET) is a distance-dependent interaction between the electronic excited states of two dye molecules in which excitation is transferred from a donor molecule to an acceptor molecule without emission of a photon. The efficiency of FRET is dependent on the inverse sixth power of the intermolecular separation, making it useful over distances comparable with the dimensions of biological macromolecules. Thus, FRET is an important technique for investigating a variety of biological phenomena that produce changes in molecular proximity. When FRET is used as a contrast mechanism, colocalization of proteins and other molecules can be imaged with spatial resolution beyond the limits of conventional optical microscopy.

Fluorescence Imaging Plate Reader (FLIPR) uses an argon laser to rapidly scan a microtiter plate containing dye loaded cells and a semi-confocal detection method. Ca²⁺ levels are measured using indicators such as fluo-3 or Calcium Green, which are efficiently excited at 488 nm wavelength of the argon laser.

Patch clamping is another technique which allows for assessment of ionic currents at the level of the whole cell membrane. A freshly made glass pipette with a tip diameter of only a few micrometers (μm) is pressed gently on the cell membrane to form a gigaseal. When suction is applied to the pipette the membrane breaks and the cytoplasm and pipette solution containing a specific agent start to mix. This is all done by monitoring the voltage changes across a membrane. As ions move from one side of the membrane to another a particular voltage is produced. In effect, the researcher can determine when ions are moving by monitoring the voltage changes.

The instant methods will find application in the treatment of a wide range of conditions associated with abnormal mechanoreceptor activation. In a particularly contemplated aspect, the present methods will be useful where the subject has a disorder associated with either an excessive or inadequate dietary intake, including, without being limited to, obesity, anorexia, bulimia, diabetes and energy imbalance, sleep apnoea, neural injury, neurological diseases, severe burns, severe trauma, chronic non-neurological diseases, chronic infections, chronic corticosteroid administration, AIDS, and the like. Neural injuries include acute brain injuries, traumatic brain injuries, closed head injuries, stroke, and the like. Neurological diseases include chronic neurodegenerative diseases such as Alzheimer's disease, Parkinson's disease, Huntington's disease, multiple sclerosis, and the like.

In a particular embodiment, the ion channel acting as a mechanoreceptor is TRPV2. Accordingly, the invention provides therapeutic compounds which are useful in the treatment or prophylaxis or modulation including control of obesity, anorexia, satiation, weight maintenance, metabolic energy levels, and/or inflammatory diseases wherein the particular compounds are selected from a calcium uptake inhibitor or promoter, a blocker or promoter of mechanoreceptor TRPV2 calcium channels and a biological dye which inhibits or promotes calcium ion flux, and salts, homologs, orthologs, analogs, isomers, derivatives or functional equivalents thereof. Calcium flux needs calcium ion uptake and release, and in particular, the movement of calcium ions into and out of the cells of the stomach wall. By calcium “flux” is included “calcium uptake” or “calcium release” which includes the movement of calcium ions into or out of cells in the stomach wall, including neuronal cells of the myenteric plexus. Such cells are particularly important for signalling a level of sensation of satiation through the vagus nerve to the brain.

In one embodiment, the cells are neuronal cells of the myenteric plexus. In another embodiment, the compounds are selected from 1-[β-[3-(4-methoxyphenyl)propoxy]-4-methoxyphenethyl]-1H-imidazole, a ruthenium red dye and salts, homologs, orthologs, analogs, isomers, enantiomers derivatives or functional equivalents thereof. In one embodiment, ruthenium dyes include ruthenium(6+), tetradecaamminedi-m-oxotri-, hexachloride, trans- (8Cl) or a stereoisomer or enantiomer thereof and/or ammoniated ruthenium oxychloride or a stereoisomer or enantiomer thereof. All such modulators of calcium flux across the cell membrane are referred to as inter alia “therapeutic” agents, compounds, medicaments or molecules. Notwithstanding that the preferred compounds block TRPV2 calcium channels, this may not necessarily be the mode of action. The invention further provides pharmaceutical compositions and methods of treatment and/or prophylaxis. Although the invention is directed to TRPV2, it also extends to TRPV1 and TRPV3 through TRPV6.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease. Generally, such a condition or disorder involves either an excessive intake of food or a deficiency in food intake.

A “subject” as used herein refers to an animal including an avian species, such as a mammal, such as a human subject, with a range of conditions associated with gastric disorders who can benefit from the pharmaceutical formulations and methods of the present invention. These gastric disorders include, without being limited to, obesity, anorexia bulimia, diabetes and/or energy imbalance, sleep apnea, neural injury, neurological diseases, inflammation, severe burns, severe trauma, chronic non-neurological diseases, chronic infections, chronic corticosteroid administration, AIDS, and the like. Neural injuries, which impact on a subject's dietary intake, include acute brain injuries, traumatic brain injuries, closed head injuries, stroke and the like. Neurological diseases include chronic neurodegenerative diseases such as Alzheimer's disease, Parkinson's disease, Huntington's disease, multiple sclerosis, and the like.

Other terms such as recipient, patient, host or target may be used in place of subject. There is no limitation on the type of animal that could benefit from the presently described pharmaceutical formulations and methods. A subject regardless of whether a human or non-human animal may be referred to as an individual, subject, animal, host or recipient. The compounds and methods of the present invention have applications in human medicine, veterinary medicine as well as in general, domestic or wild animal husbandry. For convenience, an “animal” includes an avian species such as a poultry bird, an aviary bird or game bird.

The treatment of diseases and disorders associated with inappropriate food intake are also contemplated by the methods of the present invention.

In one embodiment, the animals are humans or other primates, livestock animals, laboratory test animals, companion animals or captive wild animals.

Examples of laboratory test animals include mice, rats, rabbits, guinea pigs and hamsters. Rabbits and rodent animals, such as rats and mice, provide a convenient test system or animal model. Livestock animals include sheep, cows, pigs, goats, horses and donkeys. Non-mammalian animals such as avian species, zebrafish and amphibians (including cane toads) are also contemplated.

Accordingly, in one embodiment the invention provides compounds which modulate calcium uptake thereby influencing factors involved in obesity, anorexia, satiation, weight maintenance, metabolic energy levels, and/or inflammatory diseases. Obesity and anorexia are described with reference to a subject being lean or obese.

The terms “lean” and “obese” are used in their most general sense but should be considered relative to the standard criteria for determining obesity. Generally, for human subjects the definition of obesity is BMI>30 kg/m².

Conveniently, an animal model may be employed to study the effects of obesity. In particular, PCT/AU02/01405 exemplifies the Psammomys obesus (the Israeli sand rat) animal model of dietary-induced obesity. In its natural desert habitat, an active lifestyle and saltbush diet ensure that they remain lean and normoglycemic. However, in a laboratory setting on a diet of ad libitum chow (on which many other animal species remain healthy), a range of pathophysiological responses are seen. By the age of 16 weeks, more than half of the animals become obese and approximately one third develop diabetes and the most hyperphagic animals go on to develop hyperglycemia, highlighting the importance of excessive energy intake in the pathophysiology of obesity and diabetes in Psammomys obesu.

Psammomys obesus animals are conveniently divided into three groups viz Group A animals which are lean, normoglycemic and normoinsulinemic, Group B animals which are obese, normoglycemic and hyperinuslinemic and Group C animals which are obese, hyperglycemic and hyperinsulinemic.

The invention extends, however, to the targeting of calcium uptake in other test animals such as primates, livestock animals, laboratory test animals, companion animals or captive wild animals.

Examples of laboratory test animals include mice, rats, rabbits, guinea pigs and hamsters. Rabbits and rodent animals, such as rats and mice, provide a convenient test system or animal model. Livestock animals include sheep, cows, pigs, goats, horses, donkeys and canalids. Non-mammalian animals such as zebrafish and amphibians (including cane toads) may also be useful models.

The compounds of the invention may be manufactured and/or used in preparation, i.e. manufacture or formulation or a composition such as a medicament, pharmaceutical composition or drug. These may be administered to individuals in a method of treatment or prophylaxis. Alternatively, they may be incorporated into a patch, slow release capsule or implant or stent or other device inserted into vessels or tissue such as a catheter.

Thus, the invention extends, therefore, to a pharmaceutical composition, medicament, drug or other composition including a stent, catheter, patch or rapid or slow release formulation including an agent selected from a calcium uptake inhibitor or promoter, a blocker or promoter of TRPV2 calcium channels and a biological dye which inhibits or promotes calcium uptake. In one embodiment, the composition includes 1-[β-[3-(4-methoxyphenyl)propoxy]-4-methoxyphenethyl]-1H-imidazol and/or a ruthenium red dye and/or salts, homologs, orthologs, analogs, isomers, enantiomers, derivatives or functional equivalents thereof as well as pharmaceutical compositions comprising same. In one embodiment, ruthenium dyes are chosen from ruthenium(6+), tetradecaamminedi-m-oxotri-, hexachloride, trans- (8Cl) or a stereoisomer or enantiomer thereof and/or ammoniated ruthenium oxychloride or a stereoisomer or enantiomer thereof. In addition, the pharmaceutical composition may further contain other agent(s) for use in controlling obesity, anorexia, satiation, weight maintenance, metabolic energy levels, and/or inflammatory diseases or the other agent(s) may be in a separate composition. Another aspect of the invention contemplates a method comprising administration of such a composition to a patient such as for treatment or prophylaxis of an event or condition associated with obesity, anorexia, satiation, weight maintenance, metabolic energy levels, and/or inflammatory diseases. The compounds of the invention may also be used in the manufacture of a medicament for the treatment or prophylaxis of an event or condition associated with obesity, anorexia, satiation, weight maintenance, metabolic energy levels and inflammatory conditions.

Furthermore, the invention contemplates a method of making a pharmaceutical composition including admixing a compound of the invention with a pharmaceutically acceptable excipient, vehicle or carrier, and optionally other ingredients. Where multiple compositions are provided then such compositions may be given simultaneously or sequentially. Sequential administration includes administration within nanoseconds, seconds, minutes, hours or days. In a particular embodiment, administration is within seconds or minutes.

Such compositions are proposed to be useful in the treatment and/or prophylaxis and/or control of obesity, anorexia, satiation, weight maintenance, metabolic energy levels, and/or inflammatory diseases.

Examples of metabolic diseases include various manifestations such as diabetes and disorders associated with imbalances in metabolic energy levels. Diseases and disorders associated with genetic disorders are also contemplated by the invention.

Examples of inflammatory disease conditions contemplated by the invention include but are not limited to those diseases and disorders which result in a response of redness, swelling, pain, and a feeling of heat in certain areas that is meant to protect tissues affected by injury or disease. Inflammatory diseases which can be treated using the methods of the invention, include, without being limited to, acne, angina, arthritis, aspiration pneumonia, empyema, gastroenteritis, inflammation, intestinal flu, necrotizing enterocolitis, pelvic inflammatory disease, pharyngitis, pleurisy, raw throat, rubor, sore throat, stomach flu and urinary tract infections, Chronic Inflammatory Demyelinating Polyneuropathy, Chronic Inflammatory Demyelinating Polyradiculoneuropathy, Chronic Inflammatory Demyelinating Polyneuropathy and Chronic Inflammatory Demyelinating Polyradiculoneuropathy.

Accordingly, another aspect of the invention contemplates a method for the treatment or prophylaxis of a condition in an animal, said method comprising administering to said animal an effective amount of a compound which inhibits or promotes calcium uptake as described herein or a composition comprising same.

In a particular embodiment, the animal is a mammal such as a human or is a laboratory test animal such as a mouse, rat, rabbit, guinea pig, hamster, zebrafish or amphibian.

Agents which modulate the activation of a mechanoreceptor may also be identified by assessing the ability of potential agents to activate or decrease or prevent activation of a mechanoreceptor. Such agents may be identified in natural product collections, combinatorial, synthetic/peptide polypeptide or protein libraries or using phage display or SELEX technology. A vast range of screening methods and high through put screening methods are available.

The target polypeptide or fragment employed in such a test may either be free in solution, affixed to a solid support, or borne on a cell surface. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may measure, for example, the formation of complexes between a target or fragment and the agent being tested, or examine the degree to which the formation of a complex between a target or fragment and a known ligand is aided or interfered with by the agent being tested.

The screening procedure includes assaying (i) for the presence of a complex between the drug and the target, or (ii) an alteration in the expression levels of nucleic acid molecules encoding the target. One form of assay involves competitive binding assays. In such competitive binding assays, the target is typically labeled. Free target is separated from any putative complex and the amount of free (i.e. uncomplexed) label is a measure of the binding of the agent being tested to target molecule. One may also measure the amount of bound, rather than free, target. It is also possible to label the compound rather than the target and to measure the amount of compound binding to target in the presence and in the absence of the drug being tested.

Another technique for drug screening provides high throughput screening for compounds having suitable binding affinity to a target and is described in detail in Geysen (International Patent Publication No. WO 84/03564). Briefly stated, large numbers of different small peptide test compounds are synthesized on a solid substrate, such as plastic pins or some other surface. The peptide test compounds are reacted with a target and washed. Bound target molecule is then detected by methods well known in the art. This method may be adapted for screening for non-peptide, chemical entities. This aspect, therefore, extends to combinatorial approaches to screening for target antagonists or agonists.

Purified target can be coated directly onto plates for use in the aforementioned drug screening techniques. However, non-neutralizing antibodies to the target may also be used to immobilize the target on the solid phase. The target may alternatively be expressed as a fusion protein with a tag conveniently chosen to facilite binding and identification.

Such agents may be identified and isolated as a result of screening programs or they may be developed based on the I-D, 2-D or 3-D structure of a mechanoreceptor or a molecule which binds to a mechanoreceptor together with tests as herein described.

Following identification of a suitable agent, it may be manufactured and/or used in a preparation, i.e. in the manufacture or formulation or a composition such as a medicament, pharmaceutical composition or drug. These may be administered to individuals in a method of treatment or prophylaxis. Alternatively, they may be incorporated into a patch or slow release capsule or implant.

The terms “compound”, “active agent”, “pharmacologically active agent”, “medicament”, “active” and “drug” are used interchangeably herein to refer to a chemical compound that induces a desired pharmacological and/or physiological effect. The terms also encompass pharmaceutically acceptable and pharmacologically active ingredients of those active agents specifically mentioned herein including but not limited to salts, esters, amides, prodrugs, active metabolites, analogs and the like. When the terms “compound”, “active agent”, “pharmacologically active agent”, “medicament”, “active” and “drug” are used, then it is to be understood that this includes the active agent per se as well as pharmaceutically acceptable, pharmacologically active salts, esters, amides, prodrugs, metabolites, analogs, etc. The term “compound” is not to be construed as a chemical compound only but extends to peptides, polypeptides and proteins as well as genetic molecules such as RNA, DNA and chemical analogs thereof.

Thus, the invention extends, therefore, to a pharmaceutical composition, medicament, drug or other composition including a patch or slow release formulation comprising an agent of the present invention. The present invention also provides dietary compositions for supplementing food or water supplies for companion livestock or wild life animal population to induce or suppress appetites.

Furthermore, the invention contemplates a method of making a pharmaceutical or agricultural composition comprising admixing a compound of the instant invention with a pharmaceutically acceptable excipient, vehicle or carrier, and optionally other ingredients. Where multiple compositions are provided, then such compositions may be given simultaneously or sequentially. Sequential administration includes administration within nanoseconds, seconds, minutes, hours or days, preferably, within seconds or minutes.

Insofar as the agent is a genetic molecule, means is required to introduce a genetic molecule which is either an agent itself or encodes an agent into a target cell. Genetic molecule transfer systems known in the art may be useful in the practice of genetic manipulation. These include viral and non-viral transfer methods. A number of viruses have been used as gene transfer vectors or as the basis for preparing gene transfer vectors, including papovaviruses, adenovirus, vaccinia virus, adeno-associated virus, herpes viruses including HSV and EBV, lentiviruses, Sindbis and Semliki Forest virus and retroviruses of avian, murine and human origin.

Non-viral gene transfer methods are known in the art such as chemical techniques including calcium phosphate co-precipitation, mechanical techniques, for example, microinjection, membrane fusion-mediated transfer via liposomes and direct DNA uptake and receptor-mediated DNA transfer. Viral-mediated gene transfer can be combined with direct in vivo gene transfer using liposome delivery, allowing one to direct the viral vectors to particular cells. Alternatively, the retroviral vector producer cell line can be injected into particular tissue. Injection of producer cells would then provide a continuous source of vector particles.

In an approach which combines biological and physical gene transfer methods, plasmid DNA of any size is combined with a polylysine-conjugated antibody specific to the adenovirus hexon protein and the resulting complex is bound to an adenovirus vector. The trimolecular complex is then used to infect cells. The adenovirus vector permits efficient binding, internalization and degradation of the endosome before the coupled DNA is damaged. For other techniques for the delivery of adenovirus based vectors, see U.S. Pat. No. 5,691,198.

By “pharmaceutically acceptable” carrier, excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Similarly, a “pharmacologically acceptable” salt, ester, amide, prodrug or derivative of a compound as provided herein is a salt, ester, amide, prodrug or derivative that this not biologically or otherwise undesirable.

Liposome/DNA complexes have been shown to be capable of mediating direct in vivo genetic molecule transfer. While in standard liposome preparations the gene transfer process is non-specific, localized in vivo uptake and expression have been reported in tumor deposits, for example, following direct in situ administration.

If the genetic molecule encodes a sense or antisense polynucleotide or a ribozyme or DNAzyme, expression will produce the sense or antisense polynucleotide or ribozyme or DNAzyme. Thus, in this context, expression does not require that a protein product be synthesized. In addition to the polynucleotide cloned into the expression vector, the vector also contains a promoter functional in eukaryotic cells. The cloned polynucleotide sequence is under control of this promoter. Suitable eukaryotic promoters include those described above. The expression vector may also include sequences, such as selectable markers and other sequences described herein.

Agents are formulated in pharmaceutical compositions which are prepared according to conventional pharmaceutical compounding techniques. The composition may contain the active agent or pharmaceutically acceptable salts of the active agent. These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the form of preparation desired for administration, e.g. topical, intravenous, oral, intrathecal, epineural or parenteral.

For oral administration, the compounds can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, International Patent Publication No. WO 96/11698.

For parenteral administration, the compound may be dissolved in a pharmaceutical carrier and administered as either a solution of a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

The active agent is preferably administered in a therapeutically effective amount. The actual amount administered and the rate and time-course of administration will depend on the nature and severity of the condition being treated. Prescription of treatment, e.g. decisions on dosage, timing, etc. is within the responsibility of general practitioners or specialists and typically takes account of the disorder to be treated, the condition of the individual patient, the site of delivery, the method of administration and other factors known to practitioners. Examples of techniques and protocols can be found in Remington's Pharmaceutical Sciences, supra.

Alternatively, targeting therapies may be used to deliver the active agent more specifically to certain types of cell, by the use of targeting systems such as antibodies or cell specific ligands or specific nucleic acid molecules. Targeting may be desirable for a variety of reasons, e.g. if the agent is unacceptably toxic or if it would otherwise require too high a dosage or if it would not otherwise be able to enter the target cells.

Instead of administering these agents directly, they could be produced in the target cell, e.g. in a viral vector such as described above or in a cell based delivery system such as described in U.S. Pat. No. 5,550,050 and International Patent Publication Nos. WO 92/19195, WO 94/25503, WO 95/01203, WO 95/05452, WO 96/02286, WO 96/02646, WO 96/40871, WO 96/40959 and WO 97/12635. The vector could be targeted to the target cells. The cell based delivery system is designed to be implanted in a patient's body at the desired target site and contains a coding sequence for the target agent. Alternatively, the agent could be administered in a precursor form for conversion to the active form by an activating agent produced in, or targeted to, the cells to be treated. See, for example, European Patent Application No. 0 425 731 A and International Patent Publication No. WO 90/07936.

The following Examples are included to demonstrate particular embodiments of the invention. It should be appreciated by those of skill in the art that the techniques disclosed in the examples which follow represent techniques discovered by the inventors to function well in the practice of the invention, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Identification of Mechanoreceptors Associated with Gastric Distension

Table 3 describes 33 genes which are associated with gastric distension.

TABLE 3
Mechanoreceptors for gastric distension

| ION | | GENE | RAT | | |
|---|---|---|---|---|---|
| CHANNEL | CHR. | NAME | HOMOLOG | HOMOLOGY | REFSEQ |
| αENAC | 12p13 | SCNN1A | Scnn1a | 83 | NM_031548.1 |
| βENAC | 16p12 | SCNN1B | Scnn1b | 84 | NM_012648.1 |
| γENAC | 16p12 | SCNN1C | Scnn1c | 84 | NM_017046.1 |
| δENAC | 1p36 | SCNN1D | — | — | — |
| ASIC1 | 12q12 | ACCN2 | Accn2 | 89 | NM_024154.1 |
| ASIC2 | 17q11 | ACCN1 | Accn1 | 89 | NM_012892.1 |
| ASIC3 | 7q35 | ACCN3 | Asic3 | 82 | NM_173135.1 |
| ASIC4 | 2q36 | ACCN4 | Asic4 | 85 | NM_022234.1 |
| BLINAC/hiNaC | 4q31 | ACCN5 | Inac | 83 | NM_022227 |
| TREK1 | 1q41 | KCNK2 | Kcnk2 | 86 | NM_172042.1 |
| TREK2 | 14q31 | KCNK10 | Kcnk10 | 89 | NM_023096.2 |
| TRAAK | 11q13 | KCNK4 | LOC360318 | 83 | XM_346568.1 |
| TRPM1 | 15q13 | TRPM1 | LOC361586 | 88 | XM_341868.1 |
| TRPM2 | 21q22 | TRPM2 | LOC294329 | 83 | XM_228069.2 |
| TRPM3 | 9q21 | TRPM3 | LOC309407 | 91 | XM_219902.2 |
| TRPM4 | 19q13 | TRPM4 | Mls2s | 81 | NM_133607.1 |
| TRPM5 | 11p15 | TRPM5 | LOC365391 | 85 | XM_344979.1 |
| TRPM6 | 9q21 | TRPM6 | LOC293874 | 83 | XM_219747.2 |
| TRPM7 | 15q21 | TRPM7 | — | — | — |
| TRPM8 | 2q37 | TRPM8 | CMR1 | 87 | NM_134371.1 |
| TRPC1 | 3q23 | TRPC1 | LOC360438 | 84 | XM_346834.1 |
| TRPC2 | 11p15 | TRPC2 | Trpc2 | 85 | NM_022638.1 |
| TRPC3 | 4q27 | TRPC3 | Trpc3 | 85 | NM_021771.1 |
| TRPC4 | 13q13 | TRPC4 | Trpc4 | 87 | NM_080396.1 |
| TRPC5 | Xq23 | TRPC5 | LOC360455 | 90 | XM_346868.1 |
| TRPC6 | 11q21 | TRPC6 | Trrp6 | 89 | NM_053559.1 |
| TRPC7 | 5q31 | TRPC7 | LOC306757 | 91 | XM_225159.2 |
| TRPV1 | 17p13 | TRPV1 | Trpv1 | 85 | NM_031982.1 |
| TRPV2 | 17p11 | TRPV2 | Vr11 | 81 | NM_017207.1 |
| TRPV3 | 17p13 | TRPV3 | — | — | — |
| TRPV4 | 12q24 | TRPV4 | Trpv4 | 81 | NM_023970.1 |
| TRPV5 | 7q35 | TRPV5 | LOC360382 | 83 | XM_346696.1 |
| TRPV6 | 7q34 | TRPV6 | Trpv6 | 81 | NM_053686.1 |

### EXAMPLE 2

### Tissue Expression of Candidate Genes

Oligonucleotide primers suitable for polymerase chain reaction amplification were designed for the genes identified in Table 3. Primer sequences for each of the genes are listed in Table 4.

TABLE 4
Primer sequences for amplification of candidate ion channels

| GENE | | |
|---|---|---|
| NAME | FORWARD PRIMER | REVERSE PRIMER |
| SCNN1A | SEQ ID NO: 753 | SEQ ID NO: 754 |
| SCNN1B | SEQ ID NO: 755 | SEQ ID NO: 756 |
| SCNN1C | SEQ ID NO: 757 | SEQ ID NO: 758 |
| ACCN2 | SEQ ID NO: 759 | SEQ ID NO: 760 |
| ACCN1 | SEQ ID NO: 761 | SEQ ID NO: 762 |
| ACCN3 | SEQ ID NO: 763 | SEQ ID NO: 764 |
| ACCN4 | SEQ ID NO: 765 | SEQ ID NO: 766 |
| ACCN5 | SEQ ID NO: 767 | SEQ ID NO: 768 |
| KCNK2 | SEQ ID NO: 769 | SEQ ID NO: 770 |
| KCNK10 | SEQ ID NO: 771 | SEQ ID NO: 772 |
| KCNK4 | SEQ ID NO: 773 | SEQ ID NO: 774 |
| TRPM1 | SEQ ID NO: 775 | SEQ ID NO: 776 |
| TRPM2 | SEQ ID NO: 777 | SEQ ID NO: 778 |
| TRPM3 | SEQ ID NO: 779 | SEQ ID NO: 780 |
| TRPM4 | SEQ ID NO: 781 | SEQ ID NO: 782 |
| TRPM5 | SEQ ID NO: 783 | SEQ ID NO: 784 |
| TRPM6 | SEQ ID NO: 785 | SEQ ID NO: 786 |
| TRPM8 | SEQ ID NO: 787 | SEQ ID NO: 788 |
| TRPC1 | SEQ ID NO: 789 | SEQ ID NO: 790 |
| TRPC2 | SEQ ID NO: 791 | SEQ ID NO: 792 |
| TRPC3 | SEQ ID NO: 793 | SEQ ID NO: 794 |
| TRPC4 | SEQ ID NO: 795 | SEQ ID NO: 796 |
| TRPC5 | SEQ ID NO: 797 | SEQ ID NO: 798 |
| TRPC6 | SEQ ID NO: 799 | SEQ ID NO: 800 |
| TRPC7 | SEQ ID NO: 801 | SEQ ID NO: 802 |
| TRPV1 | SEQ ID NO: 803 | SEQ ID NO: 804 |
| TRPV2 | SEQ ID NO: 805 | SEQ ID NO: 806 |
| TRPV4 | SEQ ID NO: 807 | SEQ ID NO: 808 |
| TRPV5 | SEQ ID NO: 809 | SEQ ID NO: 810 |
| TRPV6 | SEQ ID NO: 811 | SEQ ID NO: 812 |

The candidate ion channels were tested for expression in the stomach, as well as a range of other rat tissues. The tissues were obtained from a single male Sprague-Dawley rat (Rattus norvegicus) at 20 weeks of age. The animal was killed by pentobarbitone overdose (120 mg/kg) followed by cervical dislocation. The following tissues were rapidly excised and snap frozen in liquid nitrogen: spleen, pancreas, epididymal fat, stomach, colon and brain. Total RNA was extracted from the tissues using standard protocols, and PCR was performed to detect expression of each of the candidate ion channels in each of these tissues. The results of this experiment are detailed in Table 5.

TABLE 5
Tissue expression of candidate ion channels in rats

| | | PAN- | | STOM- | | |
|---|---|---|---|---|---|---|
| GENE | SPLEEN | CREAS | FAT | ACH | COLON | BRAIN |
| Scnn1a | ✓ | | ✓ | ✓ | ✓ | |
| Scnn1b | ✓ | | ✓ | ✓ | ✓ | ✓ |
| Scnn1c | ✓ | | ✓ | ✓ | ✓ | ✓ |
| Accn2 | ✓ | | ✓ | ✓ | ✓ | ✓ |
| Accn1 | ✓ | | ✓ | ✓ | ✓ | ✓ |
| Asic3 | ✓ | | ✓ | ✓ | ✓ | ✓ |
| Asic4 | ✓ | | ✓ | ✓ | ✓ | ✓ |
| Inac | ✓ | | ✓ | ✓ | ✓ | ✓ |
| Kcnk2 | ✓ | | ✓ | ✓ | ✓ | ✓ |
| Kcnk10 | ✓ | | ✓ | ✓ | ✓ | ✓ |
| LOC360318 | ✓ | | ✓ | ✓ | ✓ | ✓ |
| LOC361586 | ✓ | | ✓ | ✓ | ✓ | ✓ |
| LOC294329 | ✓ | | ✓ | ✓ | ✓ | ✓ |
| LOC309407 | ✓ | | ✓ | ✓ | ✓ | ✓ |
| Mls2s | ✓ | | ✓ | ✓ | ✓ | ✓ |
| LOC365391 | ✓ | | ✓ | | ✓ | ✓ |
| LOC293874 | ✓ | | ✓ | ✓ | ✓ | ✓ |
| CMR1 | ✓ | | ✓ | ✓ | ✓ | ✓ |
| LOC360438 | ✓ | | ✓ | ✓ | ✓ | ✓ |
| Trpc2 | ✓ | | ✓ | ✓ | ✓ | ✓ |
| Trpc3 | ✓ | | ✓ | ✓ | ✓ | ✓ |
| Trpc4 | ✓ | | ✓ | ✓ | ✓ | ✓ |
| LOC360455 | ✓ | | ✓ | | ✓ | ✓ |
| Trrp6 | ✓ | | ✓ | ✓ | ✓ | ✓ |
| LOC306757 | | | | | | ✓ |
| Trpv1 | ✓ | | ✓ | ✓ | ✓ | ✓ |
| Vr11 | ✓ | | ✓ | ✓ | ✓ | ✓ |
| Trpv4 | | | ✓ | | ✓ | ✓ |
| LOC360382 | | | | | | |
| Trpv6 | ✓ | | ✓ | ✓ | ✓ | ✓ |

Due to the fact that they are not expressed in the stomach, and therefore could not function in detection of gastric distension, the following ion channels were excluded from further investigation: TRPM5 (LOC365391), TRPC5 (LOC360455), TRPC7 (LOC360757), TRPV4 (Trpv4) and TRPV5 (LOC360382).

### EXAMPLE 3

### Expression of Candidate Mechanoreceptors in Fasted and fed Animals

The remaining 25 candidate ion channels were tested for changes in expression after gastric distension in Sprague-Dawley rats that were fasted for 24 h or refed after fasting for 1 or 4 hours (n=5-6 in each group). Ion channel(s) activated by gastric distension demonstrate increased expression in the refed (distended stomach) compared with the fasted state (see Table 6).

TABLE 6
Expression of candidate ion channels in fasted and refed rats

| GENE | FASTED¹ | REFED 1 h¹ | REFED 4 h¹ | Max. change |
|---|---|---|---|---|
| SCNN1A | 11.3 ± 2.6 | 15.5 ± 2.7 | 17.2 ± 4.3 | ↑52% |
| SCNN1B | 15.5 ± 6.3 | 68.2 ± 18.9 | 53.0 ± 20.4 | ↑340% |
| SCNN1C | 8.7 ± 2.5 | 8.2 ± 1.6 | 6.9 ± 0.9 | ↓21% |
| ACCN2 | 11.2 ± 2.2 | 8.1 ± 2.2 | 21.8 ± 11.9 | ↑95% |
| ACCN1 | 13.0 ± 5.0 | 20.3 ± 3.1 | 22.7 ± 5.2 | ↑75% |
| ACCN3 | 30.1 ± 12.6 | 2.6 ± 0.6 | 3.6 ± 1.9 | ↓91% |
| ACCN4 | 12.8 ± 3.9 | 6.7 ± 1.8 | 6.5 ± 1.8 | ↓49% |
| ACCN5² | 32.2 ± 17.7 | 1.3 ± 0.3 | 0.7 ± SE | ↓98% |
| KCNK2 | 14.0 ± 4.2 | 10.1 ± 1.0 | 7.5 ± 1.1 | ↓46% |
| KCNK10 | 10.5 ± 1.6 | 13.4 ± 5.8 | 12.4 ± 4.2 | ↑28% |
| KCNK4² | 11.4 ± 2.3 | 11.9 ± 3.0 | 10.6 ± 2.1 | ↓7% |
| TRPM1³ | 12.5 ± 3.4 | 8.8 ± 2.8 | 5.9 ± 2.1 | ↓53% |
| TRPM2 | 34.1 ± 18.3 | 2.7 ± 0.4 | 2.8 ± 0.7 | ↓92% |
| TRPM4³ | 11.1 ± 2.4 | 13.1 ± 1.5 | 7.2 ± 0.7 | ↓35% |
| TRPM6³ | 10.5 ± 1.5 | 12.8 ± 5.6 | 11.9 ± 4.0 | ↑22% |
| TRPM8 | 11.4 ± 2.4 | 11.8 ± 2.9 | 8.9 ± 1.8 | ↓22% |
| TRPC1 | 12.5 ± 3.4 | 8.7 ± 2.8 | 6.7 ± 2.4 | ↓46% |
| TRPC3 | 10.8 ± 1.8 | 8.1 ± 1.2 | 9.6 ± 1.6 | ↓25% |
| TRPC4 | 8.4 ± 2.5 | 8.9 ± 1.7 | 7.6 ± 2.3 | ↓10% |
| TRPC6 | 1.8 ± 0.4 | 1.4 ± 0.2 | 1.2 ± 0.1 | ↓50% |
| TRPV1 | 10.9 ± 2.1 | 8.0 ± 1.0 | 5.7 ± 1.1 | ↓48% |
| TRPV2² | 11.1 ± 2.4 | 4.8 ± 0.8 | 4.5 ± 0.8 | ↓59% |
| TRPV6³ | 13.4 ± 5.3 | 10.9 ± 2.1 | 10.6 ± 1.8 | ↓21% |

¹Mean ± SEM, arbitrary units
²Genes previously replicably linked/associated with obesity phenotypes
³Genes previously linked/associated with obesity phenotypes

As shown in Table 6, a number of putative mechanoreceptors for physiological gastric distension have been identified. Of particular interest, TRPV2 gene expression was reduced by 59% (p=0.031) after re-feeding, which induced gastric distension in the rats. ACCN5 gene expression was reduced by 98% after re-feeding, its expression was virtually abolished when the stomach was distended. Both of these genes are located in regions replicably linked with obesity phenotypes, and in conjunction with this gene expression data this makes them excellent candidates for signalling physiological gastric distension and playing a role in the sensation of satiety.

A number of other genes showed differential expression after gastric distension, and are also associated with the sensation of satiety perception. These include TRPM1, TRPM4 and TRPV6 (expression reduced by 53, 35 and 21%, respectively, after re-feeding) and TRPM6 (expression increased by 22% after re-feeding). All of these genes are located in genomic regions previously linked or associated with obesity phenotypes. These genes are therefore strong candidates for the mechanotransduction of gastric distension, and may play a role in the sensation of satiety.

Of the remaining genes many demonstrated increased gene expression after re-feeding (ranging from 28-340% increase), while a number of other genes exhibited decreased expression after re-feeding (ranging from 7-92% decrease). These genes that show a substantial change in expression following gastric distension are also involved in the sensation of satiety.

As shown above, a number of ion channels that show altered expression following gastric distension have been identified, demonstrating that they are regulated in response to this mechanical stimulus. Each of these ion channels is a potential target for development of obesity treatments. Specifically, as stated above, the identification of chemical compounds that can activate or block these ion channels independent of mechanical stimuli could induce a sensation of satiety despite the fact that the stomach is not distended, and therefore could be useful in the treatment of obesity.

### EXAMPLE 4

### Genomic Location and Expression Profiles of Candidate Mechanoreceptor Genes

Many of these ion channels are of particular interest as they are located in genomic regions previously linked with obesity. This provides further evidence that they may be causally involved in the pathophysiology of the disease.

Particularly, TRPV2 is located on chromosome 17p11 in a region strongly linked with plasma leptin concentrations (LOD score=4.97) in Caucasians in the USA. This same region was linked with body mass index (BMI), another obesity phenotype, with a maximum LOD score of 2.47, in a combined analysis of white, black, Mexican and Asian Americans. Taken together, these studies appear to provide convincing evidence that either TRPV2 or a nearby gene plays a significant causal role in the development of obesity.

TRPV4 is located on chromosome 12q24. This genomic region was linked with BMI in Finnish and Swedish Caucasians (LOD score=1.85), and associated with BMI in a Caucasian population (p=0.03). Furthermore, in the Quebec Family Study, this region of chromosome 12 was linked with waist circumference (LOD score=2.88), another obesity phenotype. Taken together, these studies provide convincing evidence that either TRPV4 or a nearby gene plays a significant causal role in the development of obesity.

TRPM5 and TRPC2 are both located on chromosome 11p15 in a region linked with obesity in Mexican Americans (LOD score=1.6), and associated with BMI in French Caucasians (p=0.0032). Therefore it is highly likely that TRPM5 or TRPC2 or a nearby gene contribute to the development of obesity.

KCNK4 (TRAAK) is located on chromosome 11q13. This region was associated with obesity (p=0.006) in the Quebec Family Study, and showed evidence of linkage with BMI (LOD score=2.2) in study in Caucasian subjects. These studies support the contention that KCNK4 or a nearby gene appears to play a causal role in the development of obesity.

ACCN5 (BLINAC/hiNaC) is located on chromosome 4q31 in a region linked to BMI in both French Caucasians (LOD score=2.09) and Ashkenazi Jews (LOD score=2.41). Therefore it is highly likely that ACCN5 or a nearby gene contributes to the development of obesity.

TRPV6 is located on chromosome 7q34, a region strongly linked with BMI (LOD score=3.8) in the National Heart, Lung, and Blood Institute Family Heart Study. TRPM1 is located on chromosome 15q13, a region also linked with BMI (LOD score=1.6) in the National Heart, Lung, and Blood Institute Family Heart Study. TRPM4 is located on chromosome 19q13, a region strongly linked with BMI (LOD score=2.6) in Mexican Americans. Finally, TRPM3 and TRPM6 are both located on chromosome 9q21, a region linked with BMI (LOD score=1.7) in the Framingham population. Therefore, it appears that TRPV6, TRPM1, TRPM4, TRPM3 or TRPM6 may be involved in the development of obesity in a range of populations.

### EXAMPLE 5

### 1-[β-[3-(4-methoxyphenyl)propoxy]-4-methoxyphenethyl]-1H-imidazole

This compound is available from BIOMOL Research Laboratories (PA, USA) under catalog number CA-230 and product identification SK&F 96365.

It is a white solid with a melting point of 117-119° C. Its molecular formula is C₂₂H₂₆N₂O₃.HCl and it has a molecular weight of 402.9. Solubility in water is 30 mg/ml.

This compound is a selective inhibitor of receptor-mediated Ca²⁺ entry in stimulated platelets (IC₅₀=8-12 μM), neutrophils and endothelial cells.

### EXAMPLE 6

### Ruthenium Red

This compound is a biological dye and general inhibitor of calcium uptake. It blocks all TRPV channels. Its chemical formula is H₄₂C₁₆N₁₄O₂Ru₃4H₂O. It is available from Electron Microscopy Sciences (PA, USA) under Catalog Number 20600.

### EXAMPLE 7

### Intragastric gavage of 1-[beta-[3-(4-Methoxyphenyl)propoxy]-4-methoxyphenethyl]-1H-imidazole.HCl inhibits food intake in rats

Adult, male rats were fasted for 16 hours then administered with a single intragastric gavage of 1-[beta-[3-(4-Methoxyphenyl)propoxy]-4-methoxyphenethyl]-1H-imidazole.HCl at a dose of 5 mg/kg in saline in a volume of 0.5 ml, or saline alone (0.5 ml). There were 8 animals in each group. Food intake was measured at various timepoints over the following 24 hours. As shown in Table 7, 1-[beta-[3-(4-Methoxyphenyl)propoxy]-4-methoxyphenethyl]-1H-imidazole.HCl administration reduced food intake over the period of the study, with statistically significant effects after 2 h, 7 h, 11 h and 12 h (p<0.05, independent samples t-test). There were strong trends for reduced food intake after 3 h, 5 h, 6 h, 8 h, 9 h, 10 h and 24 h (p<0.10). Taken together, these data strongly suggest that oral administration of 1-[beta-[3-(4-Methoxyphenyl)propoxy]-4-methoxyphenethyl]-1H-imidazole HCl reduces food intake in rats, and therefore this compound has potential use as an orally administered regulator of food intake in humans.

TABLE 7
Food Intake

Food Intake (g)*

| Time (h) | Treated (n = 8) | Control (n = 8) | p-value |
|---|---|---|---|
| 0.5 | 2.4 ± 0.3 | 2.9 ± 0.2 | 0.175 |
| 1 | 2.8 ± 0.3 | 3.2 ± 0.3 | 0.295 |
| 2 | 2.9 ± 0.3 | 4.0 ± 0.3 | 0.038 |
| 3 | 3.8 ± 0.4 | 4.9 ± 0.3 | 0.062 |
| 4 | 4.9 ± 0.5 | 5.6 ± 0.3 | 0.315 |
| 5 | 5.1 ± 0.5 | 6.6 ± 0.3 | 0.055 |
| 6 | 6.3 ± 0.5 | 7.5 ± 0.3 | 0.078 |
| 7 | 7.4 ± 0.6 | 8.9 ± 0.3 | 0.035 |
| 8 | 8.2 ± 0.6 | 9.7 ± 0.3 | 0.054 |
| 9 | 9.3 ± 0.6 | 10.8 ± 0.3 | 0.093 |
| 10 | 10.3 ± 0.6 | 11.7 ± 0.3 | 0.078 |
| 11 | 11.5 ± 0.6 | 13.7 ± 0.3 | 0.021 |
| 12 | 12.4 ± 0.6 | 14.7 ± 0.3 | 0.011 |
| 24 | 18.4 ± 0.8 | 20.1 ± 0.3 | 0.087 |

*mean ± sem.

### EXAMPLE 8

### Screening for Agents to TRPV2

TRPV2 is used as a target for molecules which interact or associate with it at a site including an amino acid sequence selected from the group consisting of SEQ ID NO:1 through SEQ ID NO:752. Candidate molecules are then tested for biological activity in a cell-based system.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of said steps or features.

All of the COMPOSITIONS, and METHODS disclosed and claimed herein can be made and executed without undue experimentation in light of the present disclosure. While the compositions and methods of this invention have been described in terms of particular embodiments, it will be apparent to those of skill in the art that variations may be applied to the COMPOSITIONS, and METHODS and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

We claim:

1. A method for modulating the perception of satiety in a subject, the method comprising: administering to the subject an effective amount of an agent which modulates the level or activity of TRPV2, such that increasing or decreasing the level of or activity of TRPV2 changes the perception of satiety in the subject.

2. The method of claim 1 wherein the agent is an agonist of TRPV2 which promotes the perception of satiety.

3. The method of claim 1 wherein the agent is an antagonist of TRPV2 which reduces the perception of satiety.

4. The method of claim 1, wherein the agent interacts with or modulates the activity of a portion of TRPV2 which comprises an amino acid sequence selected from SEQ ID NO:1 through SEQ ID NO:752.

5. The method of claim 4 wherein the agent interacts with or modulates the activity of a portion of TRPV2 selected from amino acid residue numbers 483 to 491 and 558 to 621 of SEQ ID NO:753.

6. The method of claim 1 wherein the subject comprises a mammal.

7. The method of claim 6 wherein the mammal comprises one or more of a primate, a human, or a laboratory test animal.

8. The method of claim 1 wherein the agent comprises an antagonist of TRPV2 chosen from 1-[β-[3-(4-methoxyphenyl)propoxy]-4-methoxyphenethyl]-1H-imidazole, ruthenium red dye and their salts.

9. The method of claim 1 wherein the agent comprises a ruthenium red dye chosen from ruthenium (6t), tetradecaaminedi-m-oxotrihexachloride or a trans (8Cl) isomer or an enantiomer thereof, and an ammoniated ruthenium oxychloride or a steroisomer or enantiomer thereof.

10. A method for screening for an agent which modulates the levels or activity of TRPV2 in a subject, the method comprising: screening for agents which interact or associate with TRPV2 or a portion thereof comprising an amino acid sequence selected from SEQ ID NO:1 through SEQ ID NO:752.

11. The method of claim 10 wherein the agent interacts or associates with a portion of TRPV2 selected from amino acid residue numbers 483 to 491 and 558 to 621 of SEQ ID NO:752.

12. The method of claim 10 wherein the subject comprises a mammal.

13. The method of claim 12 wherein the mammal comprises a primate, a human, and a laboratory test animal.

14. A fragment of TRPV2 selected from SEQ ID NO:1 through SEQ ID NO:752.

15. The fragment of claim 14 wherein the fragment is selected from amino acid residue numbers 483 to 491 and 558 to 621 of SEQ ID NO:752.

16. A cell expressing a nucleic acid molecule encoding the fragment of claim 14.

17. A solid support carrying one or more fragments of claim 14.

18. A composition, comprising:
an agent that modulates the activity of an amino acid sequence selected from SEQ ID NO:1 through SEQ ID NO:752, wherein said modulation changes the perception of satiety in a subject.

19. An isolated nucleic acid molecule that encodes a protein having an amino acid sequence selected from SEQ ID NO:1 through SEQ ID NO:752.

20. A composition, comprising:
an agent that modulates the activity of TRPV2, wherein said modulation changes the perception of satiety in a subject.
